SEQUENCE LISTING (42 sequences)

SEQ ID NO: 1 - DNA, 3532 bp, Rattus norvegicus
  clone RHDH-009, rat insulin-like growth factor II (IGF-II)
gcaaactggacatttgcttctcctgtgagaaccttccagccttttcctgtcttcatcctcttccagccccagcggcctccttatccaacttcaggtaccaatggggatcccagtggggaagtcgatgttggtgcttctcatctctttggccttcgccttgtgctgcatcgctgcttaccgccccagcgagactctgtgcggaggggagcttgttgacacgcttcagtttgtctgttcggaccgcggcttctacttcagcaggccttcaagccgtgccaaccgtcgcagccgtggcatcgtggaagagtgctgcttccgcagctgcgacttggccctcctggagacatactgtgccacccccgccaagtccgagagggacgtgtctacctctcaggccgtacttccggacgacttccccagataccccgtgggcaagttcttcaaattcgacacctggagacagtccgcgggacgcctgcgcagaggcctgcctgccctcctgcgtgcccgccggggtcgcatgcttgccaaagagctcgaagcgttcagagaggccaagcgccaccgtcccctgatcgtgttaccacccaaagaccccgcccacgggggagcctcttcggagatgtccagcaaccatcagtgaaccaaattatgtggtaattctgcaatgtagtaccatcagtctgtgacctcctcttgagcagggacagctccatcatgtcccacactaaggtctctctgctccacttcccttcccaggtttctccccacccacccccatgccccgcctccccacatcaggctgctccccttgccccacaccatcgggcaaggggatcccagcaactcttcaaaaccaaatttgattggctctaaacaacccaattggcaccctccaaattatatatgaacattaaaaaaaaactttaaagcatatagtccctttacaacaaattggcttaagaaactccataactgataatctaaaaattaaataaccaaagaaattaattggctaaaaacatactaaaaattaattggcttaaaaacaattggcaaaaatcaaataatttggcccgcccccccccccttcatcttctttccatttagatctttagtcaaattggctcagacttggatctcagaacccaagaagaaaggaaggggacccaaaattttgcaggtagcatgtcattgcttcagtgctctctccttgtcactagtcacttttagcataatctggctgtgaacaacaatagccgcccaaactctttcttcactggtcattccatcacaaatgtcacccatgtcaccaaggggctgggtgaaggaacccaaggagaggaacagaacatgaaaactgaaaatagaacctaattggcacaagcccccagtcccaaaaatctcacttttcatacctactctaaaaagcacatgattatacccacacgtacatgcacacacacatgcacacaggcatgcatacacacacacacacacacacacacactattagatgagaacattgaaatggctgagcaacttcgattggaaccacattgcccaatccaaggcccatcttaaattccctgagcagtttgcatggtttgagctcctctctgaatccatctagtttctgctgccagtgtagagtcagtttggccagataaggagatggcactgccaagtgatacatgctacccgagtagcctgacccctaggtgtgctcctgggaggaaagatctgggggacaacccctaccccaagcacacctatgggccatctctgtcaatctcctggggagcccccactttttaggggctccccaggagactcacactgatgtggggagtgtgggaagtctggcggttggaggggtgggtggggggcagtgggggctgggtggggggaaactatgggtaggaagtggtcccagagaggtcttaggtggaacagtcaggaggaggcacaggtcaacttgcagaattactgaagaatcaggaccccaaattttctgtcaattgatctattcccctctttttatgtctggggcaggttttttccttttttttttttaatccctccttagcttttaatgcgctcataatcccattccctatgtaacgggggcagcgatcaagtaatgaatgcatcaagccatcaataccagcgagagccagtaacaccggctagagccatcaacaccggcttccaccatgtcctgctcccaaccatttatcaaccttttttttttttttatctgtctctatcgcttggcctgagttgggagtggagtctctgtggggtgctggccacgcacccacagagaaataaaaggaattgagaaggccgctacctggcctgacttctggggacagtggctggtccccagaagttctgaggagtggagggggcgtggggcagtgtcccctcaggtgttaggaaggtgctcggaggccacaaagatggggccccagctggccctgccagttgggggggaaggggatgtagatgtaagactagagaggttccatcaggcgggagcaagtggctgccttctgagcacttgggggaggtcctccccgtgcccctcagtgtcatcttgcccactcctcagcaccccatcttaccctcaggaggtctggagctctacagacctcctgggggcaaggtggggtgaggcctggagctggggaagcgaggaggctttaaagccttcagagccaggagaactgtgtacatggggttgtctgggccctggggcccgagggtctggtgagccgtagcagccactccacggtgcctaggactgcggcggggaacagggcggctggaggtttacctcacccccacttctgcttccagtgcagtccccctgcccaacagtcctactagtaatctagaggcctgaggcttctgggcccaggtgacaggactggcaccaccctgggggcggtgtgtgtcagccagccatggcacagagggttctcagcaagtgcctaaagaatgggccatttggaacattggacagaaactcaaagagtaaattgttataattggagaatatgaattggcctggtacccaaaatatctcgaggcaccctaaattacctgcccatttgactggacatccacccagtgttaatatgcctcgtgggatgggtgttttcaggggcatttgctgaccatcctctgtgtccccagatttgcagttctccccatcataggtcaccctgatgcaggcacctccctggcctcccatgcctagtgtggccctccatcttgttttgtctcttccctactgtcttcggtgggatcccctcttgggtcccccaatttgtcatcctgtgaagacttcccacgcgtcgaatgccatatgtcacctgtgccactgcccatgtcatccagcagtggccccgggtatttgccccaactcagtccttttaacatgcattttctggcaaaatccaaagcttgggttttgtttttaacctgttaacgcttgcaaacctaataaagcattcaaaatact

SEQ ID NO: 2 - protein, 180 aa, Rattus norvegicus
  RHDH-009, IGF-II
MGIPVGKSMLVLLISLAFALCCIAAYRPSETLCGGELVDTLQFVCSDRGFYFSRPSSRANRRSRGIVEECCFRSCDLALLETYCATPAKSERDVSTSQAVLPDDFPRYPVGKFFKFDTWRQSAGRLRRGLPALLRARRGRMLAKELEAFREAKRHRPLIVLPPKDPAHGGASSEMSSNHQ

SEQ ID NO: 3 - DNA, 876 bp, Rattus norvegicus
  clone RHDH-063, rat orthologue of presumptive human secretory protein
cggctcctgacctctgttcctgtgctcccgccgtcgtcctccagcgcaggcctccgggctccagctccggtgttgggtgcaggcctggtgtggtctccaaagtgactgaacaatgcagaaggacagtggcccactggttcctttacattattatggtttcggctatgcggccctggtggctactggtgggattattggctatgcaaaagcaggtagtgtgccgtccctggctgctggactcttctttgggggcctggcaggcctgggtgcctaccagctgtctcaggaccccaggaacgtgtgggttttcctagctacgtctgggactttggctggcattatggggatgagattctacaactctgggaaatttatgcctgcaggtttgatcgcgggagccagtttgctgatggttgccaaacttggacttagtatgttgagttcaccccatccgtagtagccatagccctgcgtgggctcatgatgagttgacactctccagtcctctacattaccacgctgaagagataagaacagcaaagacctacactgagcacatggaggcgaagacgtggttactatagtgaccgttcagagacggcgagtgtctgacctcagagctcacactgccttcatgcggcttgttcttgtgtcatgatgtctcgactctctgtactactacataaaggggtaaaatgttgggtgtcaacactgggggcccagagctacacgtcctgccgggaagttgtgaaatctcttggtgacatttgtgatgtgggatcttttgcacaggtctgctatgaaattatgttacggcaacattatcggtgaaaataaagttttctattaagaaaaaaaaaaaaaaaaaaaaaaaaa

SEQ ID NO: 4 - protein, 114 aa, Rattus norvegicus
  RHDH-063
MQKDSGPLVPLHYYGFGYAALVATGGIIGYAKAGSVPSLAAGLFFGGLAGLGAYQLSQDPRNVWVFLATSGTLAGIMGMRFYNSGKFMPAGLIAGASLLMVAKLGLSMLSSPHP

SEQ ID NO: 5 - DNA, 631 bp, Rattus norvegicus
  clone RHDH-068, rat melanocyte-specific expression gene 1 (msg1)
actagtgatttaggatccaatgccaactatgtcgaggcctgcactggatgtcaagggtggtaccacctctggaaaggaagatgccaaccaggagatgaactctctggcctactctaacctcggggtaaaagatcgcagggcagtgaccgtcctgcactaccccggggtcaccgcaaatggagccaaagccaatggagtgcccactagcccctctggatcatcatctccaacaggctctcctactgccaccccttctaccaaacccccatccttcaacctgcaccccacccctcacctgctggccagtatgcagcttcagaagcttaatagccagtaccaaggggctgcagcgactgctgctgctgccctggctgcaccaagccaaccaggagaggatgagcccctgctaaactggggcactgggatccaggcaggagcagggggatcacccggatcagtctctcctgctggtgcccagagccctgctatcattgattctgacccagtggatgaggaggtgctgatgtctctggtggtagaattggggctagaccgagccaatgagcttcccgagctgtggctagggcagaatgagtttgatttcactgcagattttccctctggctgctga

SEQ ID NO: 6 - protein, 203 aa, Rattus norvegicus
  RHDH-068, msg1
MPTMSRPALDVKGGTTSGKEDANQEMNSLAYSNLGVKDRRAVTVLHYPGVTANGAKANGVPTSPSGSSSPTGSPTATPSTKPPSFNLHPTPHLLASMQLQKLNSQYQGAAATAAAALAAPSQPGEDEPLLNWGTGIQAGAGGSPGSVSPAGAQSPAIIDSDPVDEEVLMSLVVELGLDRANELPELWLGQNEFDFTADFPSGC

SEQ ID NO: 7 - DNA, 3653 bp, Rattus norvegicus
  clone RHDH-098, homologue of mouse c-abl tyrosine kinase
acccccgtcaacagcctggagaaacattcctggtatcatggccctgtatctcggaatgctgctgagtatctgctgagcagcggaatcaacggcagcttcttagtgcgggagagtgagagtagccctggccagagatccatctcgctgcggtatgaagggagggtgtaccactacaggatcaacactgcctctgatggcaagctgtacgtgtcctccgagagccgcttcaacactctggctgagttagttcaccatcactccacggtggctgatggcctcatcaccacactccactacccagctcccaagcgcaacaagcccactatctacggtgtgtcccccaactacgacaagtgggaaatggagcgcaccgacatcaccatgaagcacaagttgggtggaggccagtacggggaggtgtacgagggcgtttggaagaagtacagcctcactgtggccgtgaagaccttgaaggaggacaccatggaggtggaggagttcctgaaggaagcggcggtgatgaaggagatcaaacaccctaacctggtgcagctgctaggggtgtgtacccgggaaccaccattctacataatcactgagttcatgacctatgggaacctgctggactacctgagggagtgtaaccggcaggaggtgagcgccgtggtactgctctacatggccacacagatctcatcagccatggagtacttggagaagaagaacttcatccacagagaccttgctgcccggaactgcctggtaggggaaaaccacttggtgaaggtggctgattttggcctgagcaggttgatgacaggggacacctacacggcccatgctggagccaaattccccatcaaatggaccgcacctgagagcctggcctacaacaagttctccatcaagtcggacgtgtgggcatttggagtattgctctgggagattgctacctatggcatgtcaccttacccgggaattgacctgtctcaggtttatgagctgctggaaaaagactaccgcatggagcgccctgaaggctgcccggagaaggtctacgagctcatgcgagcatgttggcagtggaacccctctgaccggccctcctttgctgaaatccaccaagcctttgaaaccatgttccaggaatccagtatctcagatgaggtggagaaggagctggggaaacgaggcacgagaggaggtgctgggagtatgctgcaggccccagagctgcccaccaagaccagaacctgcaggagagcagctgagcagaaagatgcgcctgacacccctgagctgctccacacgaagggcctgggagaaagcgatgcactggacagtgagcctgctgtatcgccactgcttcctcggaaagagcgcgggcccccagacggcagcctaaatgaagatgagcgccttctccccagagacagaaagaccaacctgttcagcgctttgatcaagaagaagaagaaaatggcgccgacgccccctaagcgcagcagttccttccgagagatggatggccagccagaccgcagaggggctagtgaggatgacagcagggaactctgcaatggaccaccagctctcacctcagacgcagcagagcctaccaagtccccaaaggccagcaatggggctggcgtccctaatggagccttccgggagccgggcaactcaggcttccgttctccccacatgtggaaaaagtccagcacactgaccgggagccgcctggctgctgccgaagaggagagcggcatgagctccagtaagcgcttcctgcgttcttgttcggcctcctgcatgccccatggggcaagggacacagagtggcggtcggtcacgctgcctcgagacctgccgtctgctggcaagcagtttgactcatccacctttggagggcacaaaagcgaaaagccagctctgcctcggaaacgcaccagtgagagcaggtctgagcaggtggccaaaagcacggcgatgccccctccccggctggtgaagaagaacgaggaggctgctgaagaaggcttcaaagacacagaatccagccctggctccagccctcccagcttgactcccaaactcctccgcaggcaggtcactgcctctccttcctctggcctctctcacaaggaagaggccaccaagggcagtgcctcaggcatggggactccggccactgcagagccagcaccccccagcaacaaagtgggcctcagcaaggcctcctctgaggagatgcgcgtaaggaggcacaagcacagctcggagtccccagggagagacaaggggcgactggctaagctcaagcctgccccgccgcctcctcctgcctgcacaggaaaagcaggcaagcccgcacagagccccagccaagaggccggggaggcaggggggcccacaaagacaaaatgcacgagtctggctatggatgctgtgaacactgaccccaccaaggccggcccacctggagaaggactgagaaagcctgtgcccccatctgtgccaaagccccagtcgacggctaagcctccagggactcccaccagcccggtctccaccccctccacagcaccagctccttcacccctggctggggaccagcagccatcttctgccgccttcatccccctcatatcaacccgtgtgtctcttaggaagacccgccagccgccagagcgcattgccagtggcaccatcaccaagggtgtggttctggacagtactgaggccctgtgccttgccatctcccggaactcagagcagatggccagccacagtgctgtactggaggctggcaagaacctgtacactttctgtgtgagctatgtggactctatccagcagatgaggaacaagtttgccttccgtgaggctatcaacaagctggagagcaacctccgagagctgcagatctgccctgccacagcctccagtgggccagctgccacccaagacttcagcaagctgctcagctctgtgaaggagatcagcgacattgtccggaggtagcagcaaccagtgtatgtcagcaagagatgttgcagttcacagggctcttgtgcctataaagatggggacaggggactggggagctggcgtctttccccaggagctttaaagagagacaagcagagcctgagggagacctggatggagcctggtggagttggctcttcctcctgtgttgtgcaccagctgccctgcacctttcctgtccagcccaggcgtcagccacctctcctcactgcctgtggatgggtctcctgctctgaagactacatctggcctgcctggccaccaggcttctcactccccggtgcctcagacccagctcccaggtcagcctggagtgctcttccctgtccttgcagaacgacctcctctgatggaccttcttgtcaccaaggcatgggagcccctgtgcttactgtacctgcacctttgatgcttacaaactgtccccgagagcctgtgctcactgtgttttcattggaagggctgtcgctttaagggtcatgaggtgctaaagccaggggcccagatgggtgggcactggaaacaggagctgggcagtgtggtctgtcacctgctctcagtatcttcagcagtgtgcccggcagatcttggacagcaagctt

SEQ ID NO: 8 - protein, 887 aa, Rattus norvegicus
  RHDH-098
MERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVSAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKRGTRGGAGSMLQAPELPTKTRTCRRAAEQKDAPDTPELLHTKGLGESDALDSEPAVSPLLPRKERGPPDGSLNEDERLLPRDRKTNLFSALIKKKKKMAPTPPKRSSSFREMDGQPDRRGASEDDSRELCNGPPALTSDAAEPTKSPKASNGAGVPNGAFREPGNSGFRSPHMWKKSSTLTGSRLAAAEEESGMSSSKRFLRSCSASCMPHGARDTEWRSVTLPRDLPSAGKQFDSSTFGGHKSEKPALPRKRTSESRSEQVAKSTAMPPPRLVKKNEEAAEEGFKDTESSPGSSPPSLTPKLLRRQVTASPSSGLSHKEEATKGSASGMGTPATAEPAPPSNKVGLSKASSEEMRVRRHKHSSESPGRDKGRLAKLKPAPPPPPACTGKAGKPAQSPSQEAGEAGGPTKTKCTSLAMDAVNTDPTKAGPPGEGLRKPVPPSVPKPQSTAKPPGTPTSPVSTPSTAPAPSPLAGDQQPSSAAFIPLISTRVSLRKTRQPPERIASGTITKGVVLDSTEALCLAISRNSEQMASHSAVLEAGKNLYTFCVSYVDSIQQMRNKFAFREAINKLESNLRELQICPATASSGPAATQDFSKLLSSVKEISDIVRR

SEQ ID NO: 9 - DNA, 1725 bp, Rattus norvegicus
  clone RHDH-099, rat non-neuronal enolase
agtgctgctccggtaccgagtcgcgctcacgtttgtccttaagactcccttcggtgtctccaggaccctctttccttcctccgcagcgatcctactgccagaacttcaccatgtccattctcaagatccatgccagagagatctttgactcccgcgggaatcccaccgttgaggtggatctctacaccgcaaaaggtctcttccgtgctgcggtgcccagcggtgcgtccactggcatctacgaggccctagaactccgagacaatgataagacccgcttcatggggaagggtgtctcaaaggctgttgagcacatcaataaaactattgcacctgctctggttagcaagaaactgaatgttgtggagcaggagaagattgaccagctgatgatcgagatggacggcacagagaataaatctaagtttggtgcaaatgccatcctgggagtgtccctggctgtctgcaaggctggtgccgtggggaagggggtgcccctttaccgtcacattgccgacttggccggcaaccctgaagtcactctgccggtcccagctttcaatgtgatcaacggcggttctcatgctggcaacaagttggccatgcaagagttcatgatcctgcctgtgggggcatcctctttccgggaagccatgcgcattggagcagaggtttaccacaacctgaagaacgtcatcaaagagaagtacgggaaagacgccaccaatgtgggtgatgagggtggattcgcacctaacatcctggagaacaaagaagcactggagctgctcaagtctgccattgcaaaggccggctacactgaccaggttgtcatcggcatggatgtggctgcctccgagttctacagggctggcaagtatgacctggacttcaagtctccagatgatgccagccggtacatcacacccgaccagctggccgacctgtacaagtccttcatcaaggactacccagtggtgtccattgaagatccctttgaccaggacgactgggatgcttggcagaagttcacagctactgcaggcatccaggtggtgggggatgacctcacagtgaccaaccctaagcggatcgccaaggctgcaggcgaaaagtcctgcaactgcctcctgctcaaagtgaaccagattggctctgtgaccgagtctctgcaggcgtgtaagctggcccagtccaatggctggggtgtcatggtgtcccatcgatctgaggagactgaggacactttcattgccgacctggtggtggggctctgcactgggcagatcaagactggtgccccctgccgatctgagcgcctggccaagtacaatcagatccttagaatcgaggaggaactgggcagcaaagccaagtttgccggcaggtccttcaggaaccccctggccaagtaaggcatggaccggagatccctggagctaccagatcctctgtctccgtcatccaggcggctcaaggctggcccagtgcttgcccctcccatgtcactgcttccttagatgtccaccccgaccacctggagccctgctggagcccccagctttgtaatcatgtgatcagtctgaatcattgtttctgtcacctgactttccagctagtgtctggagccctctgaactccagcgtaatctctagagtgcccacaccatcaagactccccccagtggtttacttgcaaaaataaaagctgcagaagctcaaaaaa

SEQ ID NO: 10 - protein, 434 aa, Rattus norvegicus
  RHDH-099, non-neuronal enolase
MSILKIHAREIFDSRGNPTVEVDLYTAKGLFRAAVPSGASTGIYEALELRDNDKTRFMGKGVSKAVEHINKTIAPALVSKKLNVVEQEKIDQLMIEMDGTENKSKFGANAILGVSLAVCKAGAVGKGVPLYRHIADLAGNPEVTLPVPAFNVINGGSHAGNKLAMQEFMILPVGASSFREAMRIGAEVYHNLKNVIKEKYGKDATNVGDEGGFAPNILENKEALELLKSAIAKAGYTDQVVIGMDVAASEFYRAGKYDLDFKSPDDASRYITPDQLADLYKSFIKDYPVVSIEDPFDQDDWDAWQKFTATAGIQVVGDDLTVTNPKRIAKAAGEKSCNCLLLKVNQIGSVTESLQACKLAQSNGWGVMVSHRSEETEDTFIADLVVGLCTGQIKTGAPCRSERLAKYNQILRIEEELGSKAKFAGRSFRNPLAK

SEQ ID NO: 11 - DNA, 5412 bp, Rattus norvegicus
  clone RHDH-231, rat receptor-linked tyrosine phosphatase (PTP-P1)
cagggcggaggctggaggccactgccaagcatggcgcccacctggagacccagcgtggtgtctgtggtgggtcctgtggggctcttccttgtactgctggccagagggtgcttggctgaagagccacccagatttatcagagagcccaaggatcagattggtgtgtcaggaggcgtggcctccttcgtgtgccaggccacaggtgaccctaagccacgggtgacctggaacaagaagggcaagaaagtgaactcacagcgctttgagaccattgactttgacgagagctcgggggccgtgctgaggatccagccacttcggacaccccgggatgagaacgtgtacgagtgtgtggcccagaactcggtgggggagatcacagttcatgcgaagctcaccgtcctgcgagaggaccagctgcctcctggcttccccaacattgacatgggcccccagttgaaggttgtagagcgcacacgcacagccaccatgctctgtgctgccagcggaaaccctgaccctgagatcacctggttcaaggacttcctgcctgtggaccccagtgccagcaatgggcggatcaagcagcttcggtcaggtgccctgcagattgagagcagcgaggagacagaccagggcaagtacgagtgtgtggccaccaaacaggcgggggtgcgctactcatcacctgccaacctctacgtgcgagtccgccgtgtggccccccgcttctccatcctgcccatgagccacgagatcatgcccggtgggaatgtgaatatcacttgtgtggctgtgggctcacccatgccctacgtgaagtggatgcagggggcagaggacctgacgcctgaggatgacatgcccgtgggtcggaatgtcctcgaactcacggatgtcaaagactcagccaactatccttgtgtggccatgtccagcctgggagtgatcgaggccgttgctgacatcactgtaaaatctctccccaaagcccctgggactcccgtggtgacggagaacactgctaccagtatcactgtcacatgggacgcaggcaatcctgaccctgtgtcctactacgtattgagtataatcaaagccaggatgggccgtatcagatcaaagaagacatcaaccaccacgcgctacagcatcggcggcctgagccccaactctgagtatgagatctgggtgtcagctgtcaactccatcggccaggcccccagtgagtcggtggtgacccgcacaggcgagcaggcaccagccagtgctcccaggaatgttcaggcgcgcatgctcagtgccaccaccatgattgtgcagtgggaggagcccgtggagcccaatggcctgatccgtggctaccgcgtctactacaccatggagcccgagcatccggtgggcaactggcagaagcacaatgtggacgacagtcttctgaccactgtgggcagcctgctagaggatgagacctacactgtgagagtgctcgccttcacatcggtgggcgatgggccactgtcagaccccatccaggtcaagacccagcagggagtgcccggccagcccatgaacttgcgggctgaggccaagtcagagaccagcattgggctctcgtggagtgcaccacggcaggagagtgtcattaagtatgaactgctcttccgggagggcgaccgaggccgagaggtggggcgaaccttcgacccaaccacagcctttgtggtggaggacctcaagcccaatacggagtacgcgttccggctggcggctcgctcgccgcagggcctgggcgccttcaccgcggttgtgcgccagcgcacactgcaggccatctcccccaagaacttcaaggtgaagatgatcatgaaaacttcagtgctgctaagctgggagttccctgacaactataactcacccacgccctacaagatccagtacaatggactcacactggacgtggatggccgcactaccaagaagctgatcacgcacctcaagccacacaccttctataacttcgtgctcaccaaccgtggcagcagcctgggaggcctgcagcagacggtcaccgccaggaccgccttcaacatgctcagtggcaagcctagtgtcgccccaaagcctgacaacgatggttccattgtggtctacctgcctgatggccagagtcccgtgacagtgcagaactacttcattgtgatggtcccacttcggaagtctcgtggtggccagttccctatcctactacctagtccagaggacatggatctggaggagctcatccaggacctctcccggctgcagagcagcctgcgccactcaagacagctggaggtgcctcggccttacatcgccgctcggttctccatcctgccagctgtcttccatcctgggaaccagaagcaatatggtggctttgacaacaggggcttggagccaggccaccgttatgtcctctttgtacttgctgtgctgcagaagaatgagcctacatttgcagccagtcccttctcagaccccttccaactggacaacccagacccgcagcccattgtggatggcgaggagggcctcatctgggtgatcgggcccgtgctggccgtggtcttcatcatctgcatcgtaattgccatcctgctgtacaagaacaagcctgacagcaaacgcaaggactcagagccccgcaccaaatgcttattgaacaatgcagacctcgccccccatcaccccaaggaccctgtggaaatgcgacgtatcaacttccagacgccaggtatgctcagccacccgcccattcccatcacagacatggctgaacacatggagagactcaaagccaacgacagcctcaagctctcccaggagtatgagtccatcgaccctggccagcagttcacttgggaacattcgaacctggaggccaacaagccaaagaaccgatacgccaatgtcatcgcctatgaccattcacgagtcatcctgcagcctttagaaggcatcatgggtagtgattacatcaatgccaactatgttgacggctatcggcggcagaacgcatacatcgccacgcaggggcccctgcctgagacctttggggacttctggcggatggtgtgggagcagcggtcagccactgtggtcatgatgacacggctggaggagaaatcacgggtcaaatgtgaccagtactggcctaaccgaggcaccgagacatacggcttcatccaggtcaccctactagatactatggagctggccaccttctgtgtcaggaccttttctctacacaagaatggctctagtgagaagcgtgaggtacgacattttcagttcacagcatggcctgaccacggggtacccgagtaccccacacccttcctggcgtttctgcgcagagtcaagacctgcaacccgcctgacgctggcccagttgtggtccactgcagcgcgggtgtggggcgtactggctgcttcattgtaattgatgccatgttggagcgcatcagaacagagaagacggtggatgtgtacggacacgtgacactcatgcggtcacagcgcaactacatggtgcagacagaggatcagtatagcttcatccacgaggcactgctggaggctgtgggctgtggcaataccgaggtccccgcgcgcagcctctacacctatatccagaagctggcccaggtggagcctggcgagcatgtcacaggaatggagcttgagttcaagaggcttgcagctccaaggcacacacttcgagattcattcactgccagcctgccttgcaacaagtttaagaaccgcctggtgaacatcctgccgtacgagagctcgcgtgtctgcctgcagcccattcgtggtgtcgagggctctgactacatcaatgccagcttcatcgacggctacagacagcagaaagcctacattgcaacgcagggtccactggcagagaccacagaggacttctggcgtgccctgtgggagaacaactccactattgtggtaatgctcaccaagctccgcgagatgggccgggagaagtgccaccagtactggccagctgagcgctctgcccgctaccagtactttgtggttgacccgatggcagagtataacatgccagagtacattctgcgtgagtttaaggtcacagatgcccgggatggccagtcccggaccgtccgacagttcacggactggccagagcagggtgcacccaagtcaggggaaggcttcattgacttcatcggccaagtgcataagaccaaggagcagtttggccaggatggccccatctcggtgcactgtagtgctggagtgggcaggaccggagtattcatcactctgagcatcgtgctggagcgaatgcgctacgagggggtggtggacattttccagacagtgaaggtgcttcggacccagcggcctgccatggtgcagacagaggatgagtaccagttctgcttccaggcggcgttggaattgggcagctttgatcattatgcaacataagccatgggccccgcaacgcctcgacccagctccaagtgccctgcatgtgagcccagccctcggtgctggtgggaggcggcccagggaggaaacctcctctccctggagacagcactgccttctaagggcacattcctcattccttctgactccaaaacgaggttccagggtggggggtagggtggagagtagaggagccactgctcccatagctggggtcacaagggacagaactctgctcccacacttccctgcctgcctgcctgtcagcaacattcttttttttcatttttttaactgtagtgtatttttcttcatcttctttttttttttaagaaaaaaaaaacaatgcgcagtcaaattttgaaaacaacgagacacgttggctctgtttgtcgctctgtggagggccaacttttcatagtaagtgtgtcgtgtggcggctctgtgcaacaactttgatggcttctgtgtgcattcttcccacatgtccccgtgtgaatggctcacgtaggttttctttttaccctttttactttttttttaaatcaatcttcagacatatcagatgtgaaggggtgatggctggagcacctgggccaggctgcaggacatggccaccaggacacagtggctggcctcactgcccagtccctgccgcaccagagagggtctttgtcctctcctgactcatgccccgcatggaggacccccgggactacggacacttggggacacgcagccccctagagcaagtgaggtctctctttgtaggagagtgggtcagcactcgtccccgcttgttttttgggcagaagcgggtgacagccctgtatgtagataaaccaagtttgtattaataaagattcgtccgacctaaaaaaaaaaaaaaaa

SEQ ID NO: 12 - protein, 1495 aa, Rattus norvegicus
  RHDH-231, PTP-P1
MAPTWRPSVVSVVGPVGLFLVLLARGCLAEEPPRFIREPKDQIGVSGGVASFVCQATGDPKPRVTWNKKGKKVNSQRFETIDFDESSGAVLRIQPLRTPRDENVYECVAQNSVGEITVHAKLTVLREDQLPPGFPNIDMGPQLKVVERTRTATMLCAASGNPDPEITWFKDFLPVDPSASNGRIKQLRSGALQIESSEETDQGKYECVATKQAGVRYSSPANLYVRVRRVAPRFSILPMSHEIMPGGNVNITCVAVGSPMPYVKWMQGAEDLTPEDDMPVGRNVLELTDVKDSANYPCVAMSSLGVIEAVADITVKSLPKAPGTPVVTENTATSITVTWDAGNPDPVSYYVLSIIKARMGRIRSKKTSTTTRYSIGGLSPNSEYEIWVSAVNSIGQAPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEAKSETSIGLSWSAPRQESVIKYELLFREGDRGREVGRTFDPTTAFVVEDLKPNTEYAFRLAARSPQGLGAFTAVVRQRTLQAISPKNFKVKMIMKTSVLLSWEFPDNYNSPTPYKIQYNGLTLDVDGRTTKKLITHLKPHTFYNFVLTNRGSSLGGLQQTVTARTAFNMLSGKPSVAPKPDNDGSIVVYLPDGQSPVTVQNYFIVMVPLRKSRGGQFPILLPSPEDMDLEELIQDLSRLQSSLRHSRQLEVPRPYIAARFSILPAVFHPGNQKQYGGFDNRGLEPGHRYVLFVLAVLQKNEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPGMLSHPPIPITDMAEHMERLKANDSLKLSQEYESIDPGQQFTWEHSNLEANKPKNRYANVIAYDHSRVILQPLEGIMGSDYINANYVDGYRRQNAYIATQGPLPETFGDFWRMVWEQRSATVVMMTRLEEKSRVKCDQYWPNRGTETYGFIQVTLLDTMELATFCVRTFSLHKNGSSEKREVRHFQFTAWPDHGVPEYPTPFLAFLRRVKTCNPPDAGPVVVHCSAGVGRTGCFIVIDAMLERIRTEKTVDVYGHVTLMRSQRNYMVQTEDQYSFIHEALLEAVGCGNTEVPARSLYTYIQKLAQVEPGEHVTGMELEFKRLAAPRHTLRDSFTASLPCNKFKNRLVNILPYESSRVCLQPIRGVEGSDYINASFIDGYRQQKAYIATQGPLAETTEDFWRALWENNSTIVVMLTKLREMGREKCHQYWPAERSARYQYFVVDPMAEYNMPEYILREFKVTDARDGQSRTVRQFTDWPEQGAPKSGEGFIDFIGQVHKTKEQFGQDGPISVHCSAGVGRTGVFITLSIVLERMRYEGVVDIFQTVKVLRTQRPAMVQTEDEYQFCFQAALELGSFDHYAT

SEQ ID NO: 13 - DNA, 1666 bp, Rattus norvegicus
  clone RHDH-249, rat TSC-22
cggcagccgagtcggattgagctgctgcagacgccaggccactccagccagcactgccgttttcacgccccggctgcagacagctaggaggctttatctagtttgaaccaggctgctggagctcgctccttccctctctttttttccacgaggctgtttttttatttggctgcaattgcatgaaatcccaatggtgtagaccagtggcgatggatctaggagtttaccaactgagacatttttcaatttctttcttgtcgtctttgctgggaactgaaaacgcttccgtgagacttgacaatagctctggtgcaagtgtggtagctatcgacaacaaaatagagcaagctatggatctggtgaaaagccatttgatgtatgcagttagagaggaagtggaggttctgaaggagcagatcaaagaactaatagagaaaaactcccaactggagcaggagaacaatctgttgaagacactggccagtccggagcagctcgcccagtttcaggcccagctgcagactggctcccctccggctaccacgcagccacaggggaccacacagccccctgcacagccagcgtcccagggctcaggatcaaccgcatagcctgctatgccccaacagaactggctgctgctgtctgaactgaacagaccgaagagatgtgctagtgagaagccgcctccagtcacccatttcattgctgtctgcgaaagagacgtgagactcacacatgctgttctcgctttctccccagtattaagcactcatatgcttttggcttgaagaaatatactagttgagtgaattaaaggttaaacagagagtgagcatggatgtaccctgtgcaacgtggcagatgtctgaggaatggtttgattgacgctgaggaggagctctgtgccttttcaaccctccccagccgcccactctactcccaagctctggggctcgcctgcatggggctcagaaggtgggctgctcctggattttgtgttctcctctccttcccttcaaagaatttgagaggccagaaacgagactgcaaagggggggatgcagtccttttacaaaaccgacaactgtcaccaaagcttataaaacaggacagtactgtccctcttttctgaaacatcagaagacacaaaactgttagtgacacaacggtgacaggtagctgggacctaggctatcttattatgaaggttgttttgcttgttgtatatttgtgtatgtagtgtaacgaatttgtacaatagaggaccgtaactactgttaggttgtacagattgaagtttagatgttccattggctgtctgaaaaggtgtggattgtccttcctagagagatctacttaaaaactgcttcgtgacaaaaaccacacctgaagaaattttaagaatttggcacagttagtcactttgtgtcacccggaatctagctgctgagtcttgcaaagtaaaccccctgttgactgatgtcagttgagctagtgaatgaatagatggagaaacgtcagtcagttgctgaggaagtggatttcccagtaggggtttctgcagctcacctgtatagtcctgcgcatgttccccacacagaacccactgtatttacctgttctacttgtcacctttcaataaagcatatcaaatgttgatacc

SEQ ID NO: 14 - protein, 143 aa, Rattus norvegicus
  RHDH-249, TSC-22
MKSQWCRPVAMDLGVYQLRHFSISFLSSLLGTENASVRLDNSSGASVVAIDNKIEQAMDLVKSHLMYAVREEVEVLKEQIKELIEKNSQLEQENNLLKTLASPEQLAQFQAQLQTGSPPATTQPQGTTQPPAQPASQGSGSTA

SEQ ID NO: 15 - DNA, 2010 bp, Rattus norvegicus
  clone RHDH-274, rat SH3p8
tcgacccacgcgtccgcgcagagctggcaagttgatttggcggtggcggcgcctacccccgcgcggaggaacgaaatcggttcggcgacgccggcggaaaccttagttcgagcgggaagcctgacagtggcaggcggcatgtcggtggcggggctgaagaagcagttctacaaggcgagccagctggtcagcgagaaagttggtggggccgaagggaccaaactggatgatgacttcagagagatggaaaagaaagtggatatcaccagtaaggccgtggcagaggtgctggtcagaaccatagaatatctgcaacctaacccagcctcgagggccaagctgactatgctgaatactgtatccaagatccggggccaagtgaagaatcctggctacccacagtcagagggtctgctgggagagtgcatggtccgccatggcaaggaactaggcggagagtccaactttggcgatgctctgctagatgcaggtgagtctatgaagcgcctggctgaggtgaaggactcactggacatcgaggtcaagcagaacttcatcgacccactgcagaacctgtgtgacaaggatctgaaggagatccagcaccacctgaagaagttggagggccgccgccttgactttgactacaagaagaagcgccagggcaagatccctgatgaggagctgcgtcaggccctagagaagtttgaggagtccaaggaggtggcggagaccagtatgcacaacctcctggagactgatattgagcaggtgagccagctctcagccctagtggatgcccagctggactaccaccggcaggcagtgcagatcctggaggagctggctgataagctgaagcgcagggtgagagaagcctcctcacgccccaggagggagttcaagcccaggccccaggagccctttgagcttggagagctggagcagcccaatgggggattcccctgtgcctcagcacccaagatcacagcttcgtcatcatttagatcaggggacaagcccaccaggacgcccagcaagagtatgccacccctggaccagccaagctgcaaggcgctgtatgactttgagccagagaacgatggcgagctgggcttccgagagggtgacctcatcacgcttaccaaccagatcgatgagaactggtatgaggggatgctgcatggccagtcaggcttcttcccacttagctatgtgcaggtgctggtgcctctgcctcagtgactgcgcttgcacactgactagcgcctgcacacgctgccagtcacagtgtggcagtagtctaatgccaaggtgctctataaacactaatgttcctccaggggagacctcttccctcctccctcagccctggggcccccccatcctaagactcagaaaggcccaccctgaggttctattacctttctggtggtggcagctcccacctatttcaacccttcccagcccgttgctggcgatgggcccatgcccctctccaggctccctaggggaggcaggtccttgggatccccagcctgcaagcacagccagctcagcatatggagacacctggcacctgctgctcattcagaagtgcacaaggcatgaacgtgtacacttcccatgggaccacagacccagctcagccctgttgaagaccaagcacaaaggccctgaagagtggacattcccaggtccctggcaccttcccctgagccagctccactgctacctgctcatgtgactctacagctggccacaggcagttggcaggtcccttttcaaccagcatgcgaggctggccacagccgcggctctgcatcatgagggaggctttggctggactcagttacactcttctctacagctgccccacaacccgtggcttgtccctggtacgtggggccacacccatgccccctagatgggcaacactgtcctccagcctgtgaagtggactttactcctaattttttttttttaaaagtattaaatatctctttctat

SEQ ID NO: 16 - protein, 368 aa, Rattus norvegicus
  RHDH-274, SH3p8
MSVAGLKKQFYKASQLVSEKVGGAEGTKLDDDFREMEKKVDITSKAVAEVLVRTIEYLQPNPASRAKLTMLNTVSKIRGQVKNPGYPQSEGLLGECMVRHGKELGGESNFGDALLDAGESMKRLAEVKDSLDIEVKQNFIDPLQNLCDKDLKEIQHHLKKLEGRRLDFDYKKKRQGKIPDEELRQALEKFEESKEVAETSMHNLLETDIEQVSQLSALVDAQLDYHRQAVQILEELADKLKRRVREASSRPRREFKPRPQEPFELGELEQPNGGFPCASAPKITASSSFRSGDKPTRTPSKSMPPLDQPSCKALYDFEPENDGELGFREGDLITLTNQIDENWYEGMLHGQSGFFPLSYVQVLVPLPQ

SEQ ID NO: 17 - DNA, 731 bp, Rattus norvegicus
  clone RHDH-286, rat orthologue of mouse retinoic acid-response protein (MK)
atcgagccggcccgtgagcgagatgcagcaccgaagtttcttccttctagcccttgttgccctcttggctgtcacgaccgcggtggccaaaaagaaagacaaggtgaagaagggcagcgagtgttcggagtggacctgggggccctgcacccccagcagcaaggactgcggcatgggtttccgcgagggtacctgtggggcccagacccagcgcatccattgcaaggtgccctgcaactggaagaaggagtttggagccgactgcaaatacaagtttgagagctggggggcgtgtgatgggagcactggcaccaaagcccgccaagggaccctgaagaaggctcggtacaatgcccagtgccaggagaccatccgcgtgaccaagccctgcacctccaagaccaagtcaaaggccaaagccaagaaaggaaaaggaaaggactgagtcaggaggccagagagtttctggcctgggacctgaacggagccctcctctcccacaggcccaagatgtaacccaccagtgccttttgtcttcctgtcagctttgtcaatcacacccttttactcctgccccctcttgctacacctagtacccaaagtggggagggacaagggattctgggaagtgagcctccccataaccccttttgtttcccccaccctgatacctgttatcgagaaatgaataaaatgaactcactttttttccaaaaaaaaaaaaaaaaaaa

SEQ ID NO: 18 - protein, 140 aa, Rattus norvegicus
  RHDH-286, MK
MQHRSFFLLALVALLAVTTAVAKKKDKVKKGSECSEWTWGPCTPSSKDCGMGFREGTCGAQTQRIHCKVPCNWKKEFGADCKYKFESWGACDGSTGTKARQGTLKKARYNAQCQETIRVTKPCTSKTKSKAKAKKGKGKD

SEQ ID NO: 19 - DNA, 1158 bp, Rattus norvegicus
  clone RHDH-057
cttggattcaccgccacggactccacactggagggtgagactgccagagcctacatgcccagacacatgtgcaaaggaacgcagagacaatctgaagccacgatgtccctctgcagggactcaggccccagagtttgctcactgcatgagagggctcaggtcccccgggttcacctcagtagcataggagcccagacaagggcagggcacaagcgactgacaccgaaagaacagtcatggtgtcacggggacagaaaggcagctggtcccagaagggaaacactggtgtctgcatgggagaacactctcggaagtccccagcttctgtcagactgctttcacccagcagcaaaggacaacctacgagtttaaggcaagtcccctgaggggagctgtgtgtgctgacataatactaagcccacaccaagggtccactgactgaagtgccatgggtaaagaaatagtcacatgtccccactcttgatagctcaggcaccgggctgtctcagcctgcagcacctctccaaaggggcccaaggcggcttctcatgctgagctccccacagcccctgccccacagtgctgggtctcagcacagggctaccactttccttgttgagagaatgttgcagtggctagtttggctggaacaacaaaacctcagctccatgccctcaaacactaagttttcagtgaaataaaagcaggaggccgggcccatgcgcaggcatgcctgttctttggatctggacactggaggatacattcataggaggccagcaagggccagcagtgcgtcccacttccctgaaacactggagtctgaaagcagcttgtctaccacacgcgtgcttgaacaacactgctgattctgacacatgctcacgcacacacaccacacagacacacagatgagcacacacagacttgctggaagggtcaaaggatgcccgttctgggcagcatggagtgtctggaagccgctttgactgttcagtgctaggtgtcagagcctccaagccagagtctctgtgggaacccttccagtaggagtgcgttgggatgggcagcaggacccagcaggtgctgtgctggttcttcatgaccagagaacggactgtgtttggtgtcgagcacgggcatctggtcagatgtcag

SEQ ID NO: 20 - protein, 123 aa, Rattus norvegicus
  RHDH-057
LGFTATDSTLEGETARAYMPRHMCKGTQRQSEATMSLCRDSGPRVCSLHERAQVPRVHLSSIGAQTRAGHKRLTPKEQSWCHGDRKAAGPRRETLVSAWENTLGSPQLLSDCFHPAAKDNLRV

SEQ ID NO: 21 - DNA, 762 bp, Rattus norvegicus
  clone RHDH-185
cgtgaccaatgctctgtgctgtgaatataatagcaaacccttcccctggttcagcatagttagtcgtaggggtttgctatgcctttcacgtacctctaggctcttaagtccctactgtttcacaagctttaatcagagcagtagtggtcacaggagaagggctggcttccagaagtagcccaggtcagccactgtcagtctctggaaagggcatagtgtctctgctcatttacctggagcagcacgacagtcgcatgcacctacaggaagcagtcaccacagagagcagatgtgagtccagccccacattttcagactgcagccagagtccttctcacgtcacacgcagccagagctacgctgctcttcagcttccacccggtcctgggtcctgcatgctgcacttgcttgctatgttggcgcagagcccagccagtacaaagccctctcctctcctcttttcttctcttccagcattcccctctctaaaatgtcaaccaaagagagcctcccctcccccgcaccccacccctgctctcagcctctttagacagtctcctctccatctcgtggcatgtctgtgaccctagagaattcatttacagtgccatacggaaccctgtattttacacacacagcaagcgatgtttaggtttatttatgatacttgatgctgtaaatgaaaataaatatggttctttataaaaaaaaaaaaaaaaaaaaaaaaaaaaaaaaaaaaaaaaaaaaaaaaaaaaaaaaaaa

SEQ ID NO: 22 - protein, 109 aa, Rattus norvegicus
  RHDH-185 ORF
MHLQEAVTTESRCESSPTFSDCSQSPSHVTRSQSYAALQLPPGPGSCMLHLLAMLAQSPASTKPSPLLFSSLPAFPSLKCQPKRASPPPHPTPALSLFRQSPLHLVACL

SEQ ID NO: 23 - DNA, 1312 bp, Rattus norvegicus
  clone RHDH-226, homologue of putative human secretory protein
ctgattgactggccccggtacccaggctatagagtggaacctgcggcggtgtgaacgcgcgcgaactttgtgtcgccgcggtctaacttcgactcggcttgctgcagcttcaggcaggatcctggcttccactatcccccctccatccaaccactcgggaactatggaggtagccgaggccaacagccccactgaggaggaggaagaggaagaggaggaagaaggagaggagccgatttcagagcccaggcctcacactcgctccaatcctgagggggctgaggaccgggccatcggggctcaggccaatgtgggtagccgcagcgagggcgaaggtgaggcagccactgcagatgatggggcggccagtgtcccgggagctgtgcccaagccctggcaggtaccagcaccagcatctgaggtccagattcgaacaccaagggtcaactgtccagagaaagtgatcatctgtctggatctttcagaggagatgtctgtgccaaagctggagtcctttaatgggtccagaacaaacgccctgaatgtgtctcagaagatggttgagatgtttgtgcgcacgaagcacaagattgacaagagccacgagtttgccttggtcgtagtgaacgacgactctgcctggttgtctggcctgacctctgacccacgtgaactctgcagctgcctgtacgacctagagacggcatcctgctccacattcaatttggaaggcctcttcagcctcatccagcagaagactgagctgccagtcacagagaatgtgcaaaccatcccacccccctacgtcgtgcgcaccatcctggtctacagccgcccaccctgccagccccagttctccttgactgagcccatgaagaagatgttccaatgtccctacttcttcttcgacatcatttacatccacagtggccctgaggaaaaggaagacgatatgagctggaaggacatgttcgccttcatgggcagtctggacaccaagggcaccagctacaagtatgcagtagcacttgctggccccgccctggagctgcacaactgcgtggccaagttgctggcccacccgctgcagaggccctgccagagccacgcgagctatagcctgctggaagaggacgaagaggccggtgaggggggaggccactgtgtgacaactccacaccgtcccatgaggagagaacccacaccctcgtcactggcacatgctcaatctcacactgtccactgtaaagtcattcttcctgggaccatttttgtcatcggaataaaagtctgaggccccagaaaaaaaaaa

SEQ ID NO: 24 - protein, 376 aa, Rattus norvegicus
  RHDH-226 ORF
MEVAEANSPTEEEEEEEEEEGEEPISEPRPHTRSNPEGAEDRAIGAQANVGSRSEGEGEAATADDGAASVPGAVPKPWQVPAPASEVQIRTPRVNCPEKVIICLDLSEEMSVPKLESFNGSRTNALNVSQKMVEMFVRTKHKIDKSHEFALVVVNDDSAWLSGLTSDPRELCSCLYDLETASCSTFNLEGLFSLIQQKTELPVTENVQTIPPPYVVRTILVYSRPPCQPQFSLTEPMKKMFQCPYFFFDIIYIHSGPEEKEDDMSWKDMFAFMGSLDTKGTSYKYAVALAGPALELHNCVAKLLAHPLQRPCQSHASYSLLEEDEEAGEGGGHCVTTPHRPMRREPTPSSLAHAQSHTVHCKVILPGTIFVIGIKV

SEQ ID NO: 25 - DNA, 1670 bp, Rattus norvegicus
  clone RHDH-235, homologue to rat and human metastasis-associated gene 1 (mta1)
gcgggcgggcgggcggacatggcggccaacatgtaccgggtcggagattatgtttactttgagaattcgtccagcaacccatacctaatcagaaggatagaggaactcaacaagactgcaagcggcaatgtggaagccaaagtagtctgcttttatagaagacgggacatctccaacacgctgataatgcttgccgataagcatgctaaagaaactgaggaagaatcagagacgacggttgaggctgacttgacggagaagcagaagcaccagctgaaacacagggagctctttctgtcccgccagtatgagtccctgcctgcaacacatatcagggggaagtgcagcgtggccctgctgaacgagacagaatcagtgctgtcataccttgacaaagaggataccttcttctactcattggtgtatgacccttccgtgaaaacattattggctgacaaaggtgaaatcagagtgggcccaaagtaccaagccgacattccagacgtgctgccggaaggcgactcagatgagagggaacaatcaaaattggaagttaaggtttgggaccccaatagtccgcttacggatcgacagattgaccagtttttagttgtagcccgtgccgtgggaacatttgcccgtgccctggattgcagcagctctgtgaggcagcccagcctgcatatgagcgcggctgcggcctcccgagacatcaccttgttccatgccatggacacgctgtataggcacggctatgacctcagcagtgccatcagtgtgctggtgccactcggagggccggtcctgtgcagggacgagatggaggagtggtctgcctctgaagccagcttattcgaagaagcactggaaaaatatggcaaagatttcaatgacatccgtcaggactttctcccatggaagtccttgactagcatcattgaatattattacatgtggaaaactactgacagatacgttcaacagaagcgcctaaaagctgcggaagccgagagcaaactgaaacaagtgtacatcccaacttacaaaccaaatcccaaccaaatctccagcagcaacggcaaggctggcactgtgaatggagctgtggggaccccgttccagccccagagcgcactcctaggacgagcctgtgagagctgctatgccacacagtctcaccagtggtattcctggggcccacctaatatgcagtgtagactctgtgcgacctgctggctgtattggaaaaagtacggaggtctgaaaatgcccacgcagacggacgaggagaaggctcccagccctgccgcagaggacccgcgcgtgagaagccacctgtcccggcaggccttgcagggcatgccggtccggaacaccgggagccccaagtcggccgtgaagacccgccaagctttcttcctccatactacgtatttcacaaaaattgctcgtcaggtctgcaaaaacaccctgcggctgcggcaggcagcgagacggccgtttgttgctattaactatgctgccattagggcagaatgtaagacgcttttcaattcttaaccttacacgttccgctcctcgccatcctctctctctccctcgctctctctttttgtttgtttgtttgcaataaacataagttcttgtgtaaaaaaaaaaaaaaaaaaaaa

SEQ ID NO: 26 - protein, 513 aa, Rattus norvegicus
  RHDH-235 ORF
MAANMYRVGDYVYFENSSSNPYLIRRIEELNKTASGNVEAKVVCFYRRRDISNTLIMLADKHAKETEEESETTVEADLTEKQKHQLKHRELFLSRQYESLPATHIRGKCSVALLNETESVLSYLDKEDTFFYSLVYDPSVKTLLADKGEIRVGPKYQADIPDVLPEGDSDEREQSKLEVKVWDPNSPLTDRQIDQFLVVARAVGTFARALDCSSSVRQPSLHMSAAAASRDITLFHAMDTLYRHGYDLSSAISVLVPLGGPVLCRDEMEEWSASEASLFEEALEKYGKDFNDIRQDFLPWKSLTSIIEYYYMWKTTDRYVQQKRLKAAEAESKLKQVYIPTYKPNPNQISSSNGKAGTVNGAVGTPFQPQSALLGRACESCYATQSHQWYSWGPPNMQCRLCATCWLYWKKYGGLKMPTQTDEEKAPSPAAEDPRVRSHLSRQALQGMPVRNTGSPKSAVKTRQAFFLHTTYFTKIARQVCKNTLRLRQAARRPFVAINYAAIRAECKTLFNS

SEQ ID NO: 27 - DNA, 994 bp, Rattus norvegicus
  clone RHDH-239
aaaaaagtcgttttggatggaagcatttcactaattgctttatttaagcatacaagggaaaagtcttatctgaccaattaggttgaagggttttattacctttaagaagccattagctgggatgaaacgctgcaagcacttgtgtcaaaacaaataaataatgctctctcaattagcagagaaagtggtacatctgttaaattagacacacattgcttcacacagtggcccagccagcaccccaatgtccccccacccccaacaaacaaaatgacagacagcaactttaagctagtcttaggaaagatcggggccccgaagaccaacagctctttccctctctccttccaccccaactctacaagagccagcagggcagcagagggaggtggtgtccagaggctgggcttcctccccaaggtgcttcccaggcacagggagagacaggtctgtgtgactgtcagcaggtacggagagtcctctttcaaaagcaattaccctgaggtattaccgtaccaggacatggacactaaaacttctcaaaggctcctcatcttccccacaagaagaggctcaacagaaaagggcagtgggactccatcgagtgggcgcataaagctcgggagcaaggtacaggccgagaatccatgccttcaggacacccaccagcccacaccgccttcaggcagaaggggacagtccctctccccacaggtccttgggacaatttctaaccaggtgagttagaggaaaatcaacccccacctctccaaaaaaatctattcagggaaataaattataaataaatagtgctccttttcttaaaaggtcactttcttcaaggctttcacaactcctcagcatgatctccacagattgcttggagcaagtgccctaatttggccagtccgcccaggagcagcctaacctcagaggctgagaggaggctgccgagtctcctcccccagttctgaagtggtagggtgggtgccgc

SEQ ID NO: 28 - protein, 158 aa, Rattus norvegicus
  RHDH-239 ORF
MTDSNFKLVLGKIGAPKTNSSFPLSFHPNSTRASRAAEGGGVQRLGFLPKVLPRHRERQVCVTVSRYGESSFKSNYPEVLPYQDMDTKTSQRLLIFPTRRGSTEKGSGTPSSGRIKLGSKVQAENPCLQDTHQPTPPSGRRGQSLSPQVLGTISNQVS

SEQ ID NO: 29 - DNA, 716 bp, Rattus norvegicus
  clone RHDH-279, homologue of mouse interferon regulatory factor 3 (mirf3)
ttccagcagacactcttttgccccgggggcctgcggctggtgggcagcacgtctgacaacgggacactgccctggcagccagtcaccctgccagaccctgaggagtttctgacagacaggcttgtgagggagtatgtgaggcaggtactcaaggggctgggcaaggggctggtgctgtggcgggcagggcagtgcctctgggcccagcgcctaggccactcgcattccttctgggccctgggtgaggagctgcttccagacagtgggagagggcctgatggagaggtccccaaggacaagaacggagtcgtgttcgacctcaggccctttgtggcagatctcattgccttcatggaaggaagcagacattccccacgatacactctgtggttctgtgtgggggaatcgtggccccaggaccagccgtgggtcaagaggcttgtgatggtcaaggttgttcctacatgtcttaaggagctgttagagatggcccgggaagggggagcctcatcactgaaaaccgtggacttgcacatctccaacagccagccgatctcccttacctctgaccagtacaaggcctgcctccaggacttggtggaagacatggacttccaggccactggagaaacctgagcccagctcagctgctccaataaagcaatttatgccaccatcacaaaaaaaaaaaaaaaaaaaaaaaaaaaaaaaaaaaa

SEQ ID NO: 30 - DNA, 787 bp, Rattus norvegicus
  clone RHDH-279-1, homologue of mouse interferon regulatory factor 3 (mirf3)
ggcaaaagcagtctgtctgatcaccagagtgggagttcgaggtgactgccttctaccgaggccgccaggtcttccagcagacactcttttgccccgggggcctgcggctggtgggcagcacgtctgacaacgggacactgccctggcagccagtcaccctgccagaccctgaggagtttctgacagacaggcttgtgagggagtatgtgaggcaggtactcaaggggctgggcaaggggctggtgctgtggcgggcagggcagtgcctctgggcccagcgcctaggccactcgcattccttctgggccctgggtgaggagctgcttccagacagtgggagagggcctgatggagaggtccccaaggacaagaacggagtcgtgttcgacctcaggccctttgtggcagatctcattgccttcatggaaggaagcagacattccccacgatacactctgtggttctgtgtgggggaatcgtggccccaggaccagccgtgggtcaagaggcttgtgatggtcaaggttgttcctacatgtcttaaggagctgttagagatggcccgggaagggggagcctcatcactgaaaaccgtggacttgcacatctccaacagccagccgatctcccttacctctgaccagtacaaggcctgcctccaggacttggtggaagacatggacttccaggccactggagaaacctgagcccagctcagctgctccaataaagcaatttatgccaccatcacaaaaaaaaaaaaaaaaaaaaaaaaaaaaaaaaaaaa

SEQ ID NO: 31 - protein, 94 aa, Rattus norvegicus
  RHDH-279-1 ORF
MEGSRHSPRYTLWFCVGESWPQDQPWVKRLVMVKVVPTCLKELLEMAREGGASSLKTVDLHISNSQPISLTSDQYKACLQDLVEDMDFQATGET

SEQ ID NO: 32 - DNA, 1570 bp, Rattus norvegicus
  clone RHDH-309, noncoding region
ctgacttgaacatatggtaggtgtgcctctgtaagaatccaggctcattctgagttgatgaaaagtcttatttggacatatgcagggtttggctccctgaacactcttggggggggggtcatctgacacagtaccatttagtgactttcccaagataagcatgagttgtgtccttgagtgaaacattggccatgcatggctgtsgtgggccattgcttttgctgctttagagcaccacggacctggctggtgctgagctggtccctacagctaatgctcggtaaactgtcaccttctggcctcctcatggcaccgggtccttcgtcttcatgtgcatgcatgacttgcagtagagcctactcactgcatgggagtccctggctagagtctgctgcctggcggcttgcagcgacggctcagtcaaggctgaaaggtctttsgccacacagtgaaaagtgaaccatagatggagcttgctgaccccactgacactacctgtgtttggtttgctgggctcttggtgctgggagtgcagcaagcacaggaggtaccctcgggaaggcggaggccttgcctttccggactcacctaggtcttgtctttcaacttgttgtagaagttacaaattattctcaaagatgtctgtctcatcatgagggaaaaggaactttcttttgttcacattcaggactttgagggtatagtgccataaaatgtagcaaagggccccgtgggccagatcagtgttacattgattctgaggttacagtgtctcccaccaaacatctgctgtaggccatgcggtgtatttgaagagggctttgggagaaggaagctttcctcttgctttaaaccagtatgtgcatcacatgcttacggcagacatttgctaccagtggagttctgtggagcctggctttgggcagctcattcttgaggaggtggcaaaacttggggaagttaggtgtgagtgttgtcaaggcctgaagccctatgatgcactgtggccctagaaaaggaagatgcagcctctgctgaccactgtgtcctctcagacttggccagctgcaggagcccaacctgatgttccctccttatcctctgtgtagtcattccttccgccccacaaagggagcgcaaacatttctcactgtgctgctgatgctctgacctggtgtgtctgacagatacatcaggctttgcagggcaaaggtgattcacctaagtgttcacacacagcaacactgaatcctacagaccaaaacccacttgtcagcaggagccgtggtccaggcccagcacctgtgtctcctaaccgaggccagctgtgttgggtgaagagagccatgccaagggtccaggctgctttagtccatgtgccctacccatttggggacagatggtttttcttgtaaacttgtggacttttgaaacctgttgactaaacagtaattaatttatatttgtgaaaaatgccactgtcctggtgatttctgatgtaaataatgttgtttatatagtatgtattaaattttcctaccattgtaaaaaaaaaaaaaaaaaaaaaaaa

SEQ ID NO: 33 - DNA, 1089 bp, Rattus norvegicus
  clone RHDH-100, rat protein kinase C receptor
ggcacgaggggtcgcggtggcagccgtgcggtgcttggctccctaagctatccggtgccatccttgtcgctgcggcgactcgcaacatctgacgccatgaccgagcaaatgacccttcgtgggaccctcaagggccataatggatgggttacacagatcgccaccactccgcagttcccggacatgatcctgtcggcgtctcgagacaagaccatcatcatgtggaagctgaccagggatgagaccaactacggcataccacaacgtgctcttcgaggtcactcccactttgttagcgatgttgtcatctcctctgatggccagtttgccctctcaggctcctgggatggaaccctacgcctctgggatctcacaacgggcactaccacgagacgatttgtcggccacaccaaggatgtgctgagcgtggctttctcctctgacaaccggcagattgtctctgggtcccgagacaagaccattaagttatggaatactctgggtgtctgcaagtacactgtccaggatgagagtcattcagaatgggtgtcttgtgtccgcttctccccgaacagcagcaaccctatcatcgtctcctgcggatgggacaagctggtcaaggtgtggaatctggctaactgcaagctaaagaccaaccacattggccacactggctatctgaacacagtgactgtctctccagatggatccctctgtgcttctggaggcaaggatggccaggctatgctgtgggatctcaatgaaggcaagcacctttacacattagatggtggagacatcatcaatgccttgtgcttcagccccaaccgctactggctctgtgctgccactggccccagtatcaagatctgggacttggagggcaagatcatggtagatgaactgaagcaagaagttatcagcaccagcagcaaggcagagccaccccagtgtacctctttggcttggtctgctgatggccagactctgtttgctggctataccgacaacttggtgcgtgtatggcaggtgactattggtacccgctaaaagtttatgacagactcttagaaataaactggctttctg

SEQ ID NO: 34 - protein, 317 aa, Rattus norvegicus
  RHDH-100, protein kinase C receptor
MTEQMTLRGTLKGHNGWVTQIATTPQFPDMILSASRDKTIIMWKLTRDETNYGIPQRALRGHSHFVSDVVISSDGQFALSGSWDGTLRLWDLTTGTTTRRFVGHTKDVLSVAFSSDNRQIVSGSRDKTIKLWNTLGVCKYTVQDESHSEWVSCVRFSPNSSNPIIVSCGWDKLVKVWNLANCKLKTNHIGHTGYLNTVTVSPDGSLCASGGKDGQAMLWDLNEGKHLYTLDGGDIINALCFSPNRYWLCAATGPSIKIWDLEGKIMVDELKQEVISTSSKAEPPQCTSLAWSADGQTLFAGYTDNLVRVWQVTIGTR

SEQ ID NO: 35 - DNA, 1317 bp, Rattus norvegicus
  clone RHDH-140, rat orthologue of mouse pigment epithelium-derived factor (PEDF)
gatgcagaccctggtgctactcctctggactggagccctgcttgggcacggcagcagccagaatgtccctgacagctctcaggattccccagcccctgacagcaccggggagcccgtagtggaggaggatgaccccttcttcaaggcccccgtgaacaagttggcagcagctgtttccaacttcggctacgatctctaccgcctgagatccggtgctgtctcaaccggcaacattctgctgtctcctctcagcgtggccacggccctctcggccctttccctgggagctgaacagcgaacagagtctgtcattcaccgggctctctactacgacttgatcaacaatcctgacatccacagcacctacaaggagctccttgcctctgttactgcccctgagaagaacttcaagagtgcctccagaattgtgtttgagaggaaacttcgagtaaaatccagctttgttgctcctctggagaagtcatatgggaccaggccccgaatcctcactggcaaccctcgcatagaccttcaggagattaacaactgggtgcaggcccagatgaaagggaaaattgcccggtctacaagggaaatgcccagtgccctcagcatcctcctccttggcgtggcttacttcaaggggcagtgggcaaccaagtttgactcgagaaagacgaccctccaggattttcacttggacgaggacaggactgtgagagtccccatgatgtcagaccctaaggccatcttacgatatggcttggactctgatctcaactgcaagattgcccagctgcctttgacaggaagcatgagtatcatcttcttcctgcccctgacggtgacccagaacttgaccatgatagaggagagcctcacctctgagttcattcatgacattgaccgagaactgaagactatccaagctgtgctgactgtccccaagctgaagctgagctatgaaggcgacgttaccaactctttgcaggacatgaagctacagtccttgtttgagtcccctgacttcagcaagattaccggcaaacctgtgaagctcacccaagtggaacacagggcagcttttgagtggaatgaggagggggcagggaccagctctaacccagacctccagcctgtccgcctcaccttcccgctcgactatcaccttaaccgaccgttcatctttgttctgagggacacggacacgggggccctcctcttcataggcagaatcctggaccccagcagcacttaattgtcccagtgcgctacagaaaaccccagagggagggaggactgattacacattccagg

SEQ ID NO: 36 - protein, 418 aa, Rattus norvegicus
  RHDH-140, PEDF
MQTLVLLLWTGALLGHGSSQNVPDSSQDSPAPDSTGEPVVEEDDPFFKAPVNKLAAAVSNFGYDLYRLRSGAVSTGNILLSPLSVATALSALSLGAEQRTESVIHRALYYDLINNPDIHSTYKELLASVTAPEKNFKSASRIVFERKLRVKSSFVAPLEKSYGTRPRILTGNPRIDLQEINNWVQAQMKGKIARSTREMPSALSILLLGVAYFKGQWATKFDSRKTTLQDFHLDEDRTVRVPMMSDPKAILRYGLDSDLNCKIAQLPLTGSMSIIFFLPLTVTQNLTMIEESLTSEFIHDIDRELKTIQAVLTVPKLKLSYEGDVTNSLQDMKLQSLFESPDFSKITGKPVKLTQVEHRAAFEWNEEGAGTSSNPDLQPVRLTFPLDYHLNRPFIFVLRDTDTGALLFIGRILDPSST

SEQ ID NO: 37 - DNA, 1462 bp, Rattus norvegicus
  clone RHDH-093
tttgctgtcttttccattgctgtctcctcagatggacgagaagtactaggaggagccaatgatggctgcctttatgtctttgaccgtgaacaaaaccggcgtactcttcagattgagtctcatgaggatgatgtgaatgcagtggcctttgctgacataagctcccagatcctgttctctgggggagacgatgccatctgcaaagtgtgggaccgacgcaccatgcgggaggatgaccccaagcctgtgggggcactggctggccaccaggatggcatcaccttcattgacagcaagggtgatgcccggtatctcatctccaactccaaagatcagaccattaagctctgggatatcagacgcttttccagccgagaaggcatggaagcgtcacgactggctgccacacagcagaactgggactatcgctggcagcaggtgcccaagaaagcctggaagaagttgaagctcccaggtgacagctccttgatgacctacagaggccatggagtgcttcacactctgatccgatgccgattctccccagcccacagcacgggccagcagttcatctacagcggctgttctactggcaaagtggttgtatatgacctcttaagcggccacattgtgaagaagctgaccaatcacaaggcctgtgtgcgtgatgtcagttggcatccctttgaagaaaagattgtcagcagttcgtgggatgggagcctacgcctgtggcagtaccgccaagctgagtacttccaggatgacatgaccgagtctgacaggaacagagtctgttccagtggccctgctccggtgccctgcccatctgtggccttttcctcacctcagtagatctgacctccagttctgtgtagggtgaattctcagcatattctctgcctcctccttcctttccccatttggggagtaagtggaagaactgctgacactgggtagtgagagacagaaacccagagtctgggcccaggctgagcctggactacccgtccccaaactgggccaagtggtttcctcatgcattcatacccagtctgcttggatttctatctctagccagaatgtggccggacatccattatctggggtgcagcttctgccaacaagagtcttgagtgttttaatcatgttctgatgttagatatggctcatagtgtagacacgaggtctaaatagacccattagccttttgcccaggtcttcacaccaagagttagattgaccaaccagggcccagtatactggcctttctttctgagatctttgagggaggacagggtgggcagggacgtgtttcctcagcaccctctggggtaggaactgtgtctgctctcatacttggcctttgaagtgaagagactggtctgatgtgtgggtctcagaactccgcagggccctacttgccattggatcctgtctttgctggtggccagcagcttca

SEQ ID NO: 38 - protein, 284 aa, Rattus norvegicus
  RHDH-093
FAVFSIAVSSDGREVLGGANDGCLYVFDREQNRRTLQIESHEDDVNAVAFADISSQILFSGGDDAICKVWDRRTMREDDPKPVGALAGHQDGITFIDSKGDARYLISNSKDQTIKLWDIRRFSSREGMEASRLAATQQNWDYRWQQVPKKAWKKLKLPGDSSLMTYRGHGVLHTLIRCRFSPAHSTGQQFIYSGCSTGKVVVYDLLSGHIVKKLTNHKACVRDVSWHPFEEKIVSSSWDGSLRLWQYRQAEYFQDDMTESDRNRVCSSGPAPVPCPSVAFSSPQ

SEQ ID NO: 39 - DNA, 23 bp, Artificial Sequence
  Description of Artificial SequenceT3 HT PCR primer, PCR primer specific to vector sequence
aattaaccctcactaaagggaac

SEQ ID NO: 40 - DNA, 22 bp, Artificial Sequence
  Description of Artificial SequenceT7 PCR primer
gtaatacgactcactatagggc

SEQ ID NO: 41 - DNA, 22 bp, Artificial Sequence
  Description of Artificial SequencePCR primer specific to clone RHDH-279
tcacatactccctcacaagcct

SEQ ID NO: 42 - DNA, 17 bp, Artificial Sequence
  Description of Artificial Sequenceprimer extension primer
gtaaaacgacggccagt

(19) United States
(12) Patent Application Publication
(10) Pub. No.: US 2004/0005578 A1
(43) Pub. Date: Jan. 8, 2004

(54) Myocardial cell proliferation-associated genes

(76) Inventors: Yoji Yamada, Tokyo (JP); Susumu Sekine, Tokyo (JP); Yasuhiro Kikuchi, Tokyo (JP); Kazuhiro Sakurada, Tokyo (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/258,666

(22) PCT Filed: Apr. 27, 2001
(86) PCT No.: PCT/JP01/03700

(30) Foreign Application Priority Data
Apr. 27, 2000 (JP) 2000-126741

(51) Int. Cl. C12Q 1/68; C07H 21/04

(52) U.S. Cl. 435/6; 536/23.2

(57) ABSTRACT

Genes showing different expression levels between fetal heart and adult heart were obtained. Thus, proteins useful in searching for therapeutic agents for repairing tissues injured by myocardial degeneration, DNAs encoding these proteins, antibodies recognizing these proteins, and methods of using the same are provided.

### TECHNICAL FIELD

The present invention relates to DNAs (e.g., cDNAs) that are complementary to mRNAs whose expression levels vary between fetal heart and adult heart, and which were obtained by subtraction and differential hybridization, as well as proteins encoded by the DNAs. The present invention also relates to antibodies against the proteins, methods for detecting the proteins and DNAS, and diagnostic and therapeutic agents, which comprise such DNAs, proteins, or antibodies, for various heart diseases caused by myocardial degeneration, such as hypercardia and cardiac failure.

### BACKGROUND ART

The heart is differentiated earliest among organs, at a very early stage of ontogeny. Immediately after differentiation, the heart starts to spontaneous beat. Even after differentiation, the myocardial cell maintains its proliferation potential and actively divides and proliferates during the fetal period. Specifically, a feature of the myocardial cell during this period is the occurrence of mitotic division despite the presence of many contraction filament bundles in the cytoplasm.

Most other somatic cells lose their division potential after the formation of specific cytoplasmic structures through differentiation. However, this general rule does not apply to myocardial cells.

After birth, the proliferation potential of myocardial cell decreases rapidly. Thus, the growth of heart is achieved by physiological auxesis, wherein the volume of individual myocardial cells increases. It is considered that postnatal myocardial cells have no ability to regenerate.

When myocardial cells necrose due to cardiac infarction, myocarditis, aging, etc., remaining myocardial cells adapt themselves to the situation not by cell division but by cell auxesis. Cardiomegaly occurring immediately after birth is a physiological adaptation; conversely, cardiomegaly occurring after necrosis of myocardial cells is combined with hyperplasia of coexisting cardiac fibroblast cells and interstitial fibrosis, and results in impaired diastolic function of the heart followed by impaired systolic function, which ultimately leads to cardiac failure. Symptomatic treatments, such as that reducing blood pressure and load of the volume, using agents enhancing cardiac contractile force and vasodilators, and that reducing blood volume using diuretics have been conducted as methods to treat cardiac failure caused by cardiac infarction and such. Prognosis is unfavorable in serious cardiac failure, and the heart transplantation is the only radical therapy. However, there are problems associated with transplant, including the shortage of organ donors, difficulty of brain death diagnosis, rejection, rising medical cost and such. Thus, heart transplantation has not established as a general therapy.

Cardiac failure may be treated or prevented by conferring proliferation potential to adult myocardial cells. The molecular mechanism associated with the loss of proliferation capacity of myocardial cells after birth remains to be clarified. However, molecules that are highly expressed in fetal or adult myocardial cells are presumed to be associated with the suppression of proliferation of myocardial cells, due to the different properties between fetus and adult.

Proteins whose expression levels differ between fetal heart and adult heart, include the following:

Proteins known to be more highly expressed in fetal heart than in adult heart include PCNA (proliferating cell nuclear antigen), Rb (retinoblastoma), Cyc (cyclin) D1, CycD3, and Cdk (cyclin D-dependent kinase) 4, which are involved in DNA replication or cell cycle (Am. J. Physiol., 271, H2183-H2189 (1996)). On the other hand, proteins known to be more highly expressed in adult heart than in fetal heart include Gax (Growth arrest-specific homeobox) (Am. J. Physiol., 271, H2183-H2189 (1996)).

However, for example, in spite of the fact that multiple nuclei were observed in adult myocardial cell of transgenic mice wherein forced expression of cyclin D1 was induced in a myocardial cell-specific manner, expression of only adult-specific contractile proteins was detected and no marked increase of the cell count was observed (J. Clinical Investigation, 99, 2644-2654 (1997)). Finally, proliferation potential has not yet successfully been conferred to adult myocardial cells using those proteins alone.

Accordingly, identification of new factors whose expression levels differ between fetal heart and adult heart and that may be associated with myocardial cell proliferation is required.

### DISCLOSURE OF THE INVENTION

The elucidation of the molecular mechanism for the postnatal loss of proliferation potential of myocardial cells, which possess proliferation potential during fetal period, may clarify the onset mechanism and therapeutic targets of various heart diseases caused by myocardial necrosis, and may further enable the development of a method for regenerating myocardial cells. The objectives of the present invention are: to obtain genes whose expression levels differ between fetal heart and adult heart; and to provide proteins useful in screening therapeutic agents capable of healing tissues damaged by myocardial necrosis, DNAs encoding the proteins, and antibodies recognizing the proteins, as well as uses thereof.

The present inventors persistently researched to achieve the above-mentioned objectives, and obtained following results. The inventors constructed a subtracted library enriched with genes highly expressed in the fetal heart by subtracting mRNAs extracted from the heart of an 8-week-old rat from a cDNA library constructed using, as a template, mRNAs derived from the heart of a 16-day-old fetal rat. During the construction of the subtracted library, genes whose expression levels are low are equalized and the population of vectors without insert fragment increases. Therefore, differential hybridization for each clone in the subtracted library was carried out to obtain many clones of genes whose expression levels differ between fetal heart and adult heart. The resulting clones comprised known genes whose expression levels had not previously been known to differ between fetal heart and adult heart, and novel genes as well as genes whose expression levels had been known to differ between fetal heart and adult heart. The difference of the gene expression levels was verified for these genes by Northern hybridization. Further, the present inventors identified peptides encoded by the genes and completed the present invention.

Hereinafter, a gene whose expression level differs between fetal heart and adult heart is referred to as a myocardial cell proliferation-associated gene.

The present invention provides:

(1) a DNA comprising the nucleotide sequence selected from the group consisting of the nucleotide sequences represented by SEQ ID NOs: 21, 23, 25, 27, and 30;

(2) a DNA of a gene that hybridizes, under stringent conditions, to a DNA consisting of the nucleotide sequence represented by SEQ ID NO: 21 or 27, and whose expression level varies between fetal heart and adult heart;

(3) a DNA of a gene that hybridizes under stringent conditions to a DNA consisting of the nucleotide sequence represented by SEQ ID NO: 23, 25 or 30, having a 90% or higher homology to the DNA, and whose expression level differs between fetal heart and adult heart;

(4) a DNA comprising a sequence that is identical to 5 to 60 consecutive nucleotide residues of the nucleotide sequence selected from the group consisting of the nucleotide sequences represented by SEQ ID NOs: 21, 23, 25, 27 and 30;

(5) a DNA comprising a sequence complementary to the DNA according to (4);

(6) a method for detecting mRNA corresponding to a gene whose expression level varies between fetal heart and adult heart using the DNA according to any one of (1) to (5);

(7) a diagnostic agent for heart diseases caused by myocardial degeneration, which agent comprises the DNA according to any one of (1) to (5);

(8) a method for detecting a causative gene of a heart disease caused by myocardial degeneration using the DNA according to any one of (1) to (5);

(9) a method of screening for a substance suppressing or enhancing transcription or translation of a gene whose expression level varies between fetal heart and adult heart using the DNA according to any one of (1) to (5);

(10) a method of screening for a therapeutic agent of a heart disease caused by myocardial degeneration using the DNA according to any one of (1) to (5);

(11) a therapeutic agent for a heart disease caused by myocardial degeneration, wherein the agent comprises the DNA according to any one of (1) to (5);

(12) a recombinant viral vector comprising the DNA according to any one of (1) to (5);

(13) a recombinant viral vector comprising an RNA having a sequence homologous to the sense strand of the DNA according to any one of (1) to (5);

(14) a DNA having the nucleotide sequence selected from the group consisting of the nucleotide sequences represented by SEQ ID NOs: 19, 32, and 37;

(15) a DNA of a gene hybridizing under stringent conditions to the DNA according to (14),and whose expression level varies between fetal heart and adult heart;

(16) a DNA comprising a sequence that is identical to 5 to 60 consecutive nucleotide residues of the nucleotide sequence selected from the group consisting of the nucleotide sequences represented by SEQ ID NOs: 19, 32, and 37;

(17) a DNA comprising a sequence complementary to the DNA of (16);

(18) a diagnostic agent for a heart disease caused by myocardial degeneration, wherein the agent comprises the DNA according to any one of (14) to (16);

(19) a method for detecting a causative gene of a heart disease caused by myocardial degeneration using the DNA according to any one of (14) to (16);

(20) a method of screening for a substance suppressing or enhancing transcription or translation of a gene whose expression level varies between fetal heart and adult heart using the DNA according to any one of (14) to (16);

(21) a method of screening for a therapeutic agent of a heart disease caused by myocardial degeneration using the DNA according to any one of (14) to (16);

(22) a method for detecting mRNA corresponding to a gene whose expression level varies between fetal heart and adult heart using a DNA having the nucleotide sequence selected from the group consisting of the nucleotide sequences represented by SEQ ID NOs: 1, 3, 5, 7, 9, 11, 13, 15, 17, 33, and 35;

(23) a diagnostic agent for a heart disease caused by myocardial degeneration, wherein the agent comprises a DNA comprising the nucleotide sequence selected from the group consisting of the nucleotide sequences represented by SEQ ID NOs: 1, 3, 5, 7, 9, 11, 13, 15, 17, 33, and 35;

(24) a method of screening for a substance suppressing or enhancing transcription or translation of a gene whose expression level varies between fetal heart and adult heart using a DNA comprising the nucleotide sequence selected from the group consisting of the nucleotide sequences represented by SEQ ID NOs: 1, 3, 5, 7, 9, 11, 13, 15, 17, 33, and 35;

(25) a method of screening for a therapeutic agent of a heart disease caused by myocardial degeneration using a DNA comprising the nucleotide sequence selected from the group consisting of the nucleotide sequences represented by SEQ ID NOs: 1, 3, 5, 7., 9, 11, 13, 15, 17, 33, and 35;

(26) a therapeutic agent for a heart disease caused by myocardial degeneration, wherein the agent comprises a DNA comprising the nucleotide sequence selected from the group consisting of the nucleotide sequences represented by SEQ ID NOs: 1, 3, 5, 7, 9, 11, 13, 15, 17, 33, and 35;

(27) a recombinant viral vector comprising a DNA having the nucleotide sequence selected from the group consisting of the nucleotide sequences represented by SEQ ID NOs: 1, 3, 5, 7, 9, 11, 13, 15, 17, 33, and 35;

(28) a recombinant viral vector comprising an RNA having a sequence homologous to the sense strand of a DNA comprising the nucleotide sequence selected from the group consisting of the nucleotide sequences represented by SEQ ID NOs: 1, 3, 5, 7, 9, 11, 13, 15, 17, 33, and 35;

(29) a protein comprising the amino acid sequence selected from the group consisting of the amino acid sequences represented by SEQ ID NOs: 22, 24, 26, 28, and 31;

(30) a protein having an amino acid sequence wherein one or more amino acids are deleted, substituted or added in the amino acid sequence selected from the group consisting of the amino acid sequences represented by SEQ ID NOs: 22, 24, 26, and 28, and which has an activity related to the healing of a heart disease caused by myocardial degeneration;

(31) a DNA encoding the protein according to (29) or (30);

(32) a recombinant DNA that is obtained by inserting the DNA according to any one of (1) to (4), and (31) into a vector;

(33) a transformant obtained by introducing the recombinant DNA according to (32) into a host cell;

(34) a method for producing the protein, comprising the steps of culturing the transformant according to (33), producing and accumulating the protein according to (29) or (30) in the culture, and recovering the protein from the culture;

(35) a therapeutic agent for a heart disease caused by myocardial degeneration, which agent comprises the protein according to (29) or (30);

(36) a method of screening for a therapeutic agent for a heart disease caused by myocardial degeneration comprising the steps of culturing the transformant according to (33), and screening the agent using the obtained culture;

(37) a method of screening for a therapeutic agent for a heart disease caused by myocardial degeneration using the protein according to (29) or (30);

(38) a recombinant viral vector associated with the production of the protein according to (29) or (30);

(39) a therapeutic agent for a heart disease caused by myocardial degeneration, wherein the agent comprises the recombinant viral vector according to (38);

(40) an antibody recognizing the protein according to (29) or (30);

(41) an immunological method for detecting the protein of (29) or

(30) using the antibody according to (40);

(42) a method of screening for a therapeutic agent for a heart disease caused by myocardial degeneration using the antibody according to (40);

(43) a method of screening for a substance suppressing or enhancing transcription or translation of a gene whose expression level varies between fetal heart and adult heart using the antibody according to (40);

(44) a diagnostic agent for a heart disease caused by myocardial degeneration, wherein the agent comprises the antibody according to (40);

(45) a therapeutic agent for a heart disease caused by myocardial degeneration, wherein the agent comprises the antibody according to (40);

(46) a drug delivery method for delivering to a cardiac lesion a fusion antibody in which the antibody according to (40) is bound to an agent selected from the group consisting of a radioisotope, a protein, and a low-molecular-weight compound;

(47) an antibody recognizing a protein comprising the amino acid sequence represented by SEQ ID NO: 20 or 38;

(48) a method of screening for a therapeutic agent for a heart disease caused by myocardial degeneration using the antibody according to (47);

(49) a method of screening for a substance suppressing transcription or translation of a gene whose expression level varies between fetal heart and adult heart using the antibody according to (47);

(50) a diagnostic agent for a heart disease caused by myocardial degeneration, wherein the agent comprises the antibody according to (47);

(51) a therapeutic agent for a heart disease caused by myocardial degeneration, wherein the agent comprises the antibody according to (47);

(52) a drug delivery method for delivering to a cardiac lesion a fusion antibody in which the antibody according to (47) is bound to an agent selected from the group consisting of a radioisotope, a protein, and a low-molecular-weight compound;

(53) a recombinant viral vector associated with the production of a protein comprising the amino acid sequence selected from the group consisting of the amino acid sequences represented by SEQ ID NOs: 2, 4, 6, 8, 10, 12, 14, 16, 18, 34, and 36;

(54) a therapeutic agent for a heart disease caused by myocardial degeneration, which agent comprises the recombinant viral vector according to (53);

(55) a method of screening for a therapeutic agent for a heart disease caused by myocardial degeneration using an antibody that recognizes a protein comprising the amino acid sequence selected from the group consisting of the amino acid sequences represented by SEQ ID NOs: 2, 4, 6, 8, 10, 12, 14, 16, 18, 34, and 36;

(56) a method of screening for a substance suppressing or enhancing transcription or translation of a gene whose expression level varies between fetal heart and adult heart using an antibody that recognizes a protein comprising the amino acid sequence selected from the group consisting of the amino acid sequences represented by SEQ ID NOs: 2, 4, 6, 8, 10, 12, 14, 16, 18, 34, and 36;

(57) a diagnostic agent for a heart disease caused by myocardial degeneration, wherein the agent comprises an antibody that recognizes a protein comprising the amino acid sequence selected from the group consisting of the amino acid sequences represented by SEQ ID NOs: 2, 4, 6, 8, 10, 12, 14, 16, 18, 34, and 36;

(58) a therapeutic agent for a heart disease caused by myocardial degeneration, wherein the agent comprises an antibody that recognizes a protein comprising the amino acid sequence selected from the group consisting of the amino acid sequences of SEQ ID NOs: 2, 4, 6, 8, 10, 12, 14, 16, 18, 34, and 36; and

(59) a drug delivery method for delivering to a cardiac lesion a fusion antibody in which an antibody recognizing a protein comprising the amino acid sequence selected from the group consisting of the amino acid sequences represented by SEQ ID NOs: 2, 4, 6, 8, 10, 12, 14, 16, 18, 34, and 36, is bound to an agent selected from the group consisting of a radioisotope, a protein, and a low-molecular-weight compound.

The present invention is described below in detail.

The DNAs of the present invention are DNAs of genes whose expression levels vary between fetal heart and adult heart, and include, for example, a DNA having the nucleotide sequence selected from the group consisting of the nucleotide sequences represented by SEQ ID NOs: 21, 23, 25, 27, and 30; and a DNA hybridizing, under stringent conditions, to any of the above DNA and whose expression level varies between fetal heart and adult heart.

The above-mentioned DNA that hybridizes to the nucleotide sequence selected from the group consisting of the nucleotide sequences represented by SEQ ID NOs: 21, 23, 25, 27, and 30, under stringent conditions, refers to DNAs that can be-obtained by colony hybridization, plaque hybridization, Southern blot hybridization or the like using, as a probe, a DNA having the nucleotide sequence selected from the group consisting of the nucleotide sequences of SEQ ID NOs: 21, 23, 25, 27, and 30. Specifically, such DNAs include DNAs that can be identified by immobilizing DNAs derived from bacterial colony or phage plaque on a filter, carrying out hybridization with a labeled-DNA probe in the presence of. 0.7 to 1.0 M sodium chloride at 65° C., and washing the filter with a solution of 0.1 to 2×SSC (1×SSC solution contains 150 mM sodium chloride and 15 mM sodium citrate) at 65° C.

Such hybridization can be performed according to the methods described in “Molecular Cloning, A Laboratory Manual, Second Edition, Cold Spring Harbor Laboratory Press (1989)” (hereinafter abbreviated as “Molecular Cloning 2nd Ed.”), “Current Protocols in Molecular Biology, John Wiley & Sons (1987-1997)” (hereinafter abbreviated as “Current Protocols in Molecular Biology”), “DNA cloning 1: Core Techniques, A Practical Approach, Second Edition, Oxford University (1995),” etc. Specifically, the hybridizable DNA includes a DNA having at least 60% or higher homology, preferably 80% or higher homology, more preferably 90% or higher homology to the nucleotide sequence selected from the group consisting of the nucleotide sequences of SEQ ID NOs: 21, 23, 25, 27, and 30.

Further, the DNAs of the present invention also include oligonucleotides and antisense oligonucleotides having a partial nucleotide sequence of DNAs of the present invention. Said oligonucleotides include, for example, oligonucleotides having the same sequence as the nucleotide sequence of 5 to 60 consecutive residues, preferably 10 to 40 consecutive residues of the nucleotide sequence selected from the group consisting of the nucleotide sequences of SEQ ID NOs: 21, 23, 25, 27, and 30. And such antisense oligonucleotides include, for example, antisense oligonucleotides complementary to the oligonucleotides.

The proteins of the present invention include proteins having an activity associated with heart diseases caused by myocardial degeneration. Specifically, said proteins include proteins having the amino acid sequence selected from the group consisting of the amino acid sequences of SEQ ID NOs: 22, 24, 26, 28, and 31; and proteins having an amino acid sequence wherein one or more amino acids are deleted, substituted, or added in the amino acid sequence selected from the group consisting of the amino acid sequences of SEQ ID NOs: 22, 24, 26, and 28, and having an activity associated with the healing of heart diseases caused by myocardial degeneration.

Such proteins, having an amino acid sequence wherein one or several amino acids are deleted, substituted, or added in the amino acid sequence selected from the group consisting of the amino acid sequences of SEQ ID NOs: 22, 24, 26, and 28, and having an activity associated with the healing of heart diseases caused by myocardial degeneration, can be prepared following the methods described in Molecular Cloning 2nd Ed.; Current Protocols in Molecular Biology; Nucleic Acids Research, 10, 6487 (1982); Proc. Natl. Acad. Sci., USA, 79, 6409 (1982); Gene, 34, 315 (1985); Nucleic Acids Research, 13, 4431 (1985); Proc. Natl. Acad. Sci., USA, 82, 488 (1985), etc.

1. Preparation of Myocardial Cell Proliferation-Associated Genes

(1) Preparation of Subtracted cDNA Library from Rat Heart and Selection of cDNAs from the Library by Differential Hybridization:

DNAs of myocardial cell proliferation-associated genes are prepared as follows:

First, a cDNA library is prepared from the heart of 16-day-old fetal rat by subtracting mRNAs from the heart of 8-week-old rat. Then, differential hybridization is carried out for cDNA clones of the subtracted cDNA library using RNAs from the heart of either the a 16-day-old fetal rat or an 8-week-old rat as probes. Myocardial cell proliferation-associated genes can be obtained by selecting cDNA clones whose expression levels vary between the heart of the 16-day-old fetal rat and that of the 8-week-old rat.

Subtraction is a method for selecting cDNAs of genes whose expression level vary in a control group, wherein single-stranded cDNAs are prepared from mRNAs that are extracted from tissues or cells of a certain state, the cDNAs are hybridized to mRNAs from cells of the control group, and cDNAs hybridizing to the mRNAs are subtracted.

(1)-1. Preparation of Subtracted cDNA Library

There are several methods for preparing subtracted cDNA library. In the present invention, a method wherein, first, a cDNA library is prepared by an usual method from the heart of a 16-day-old fetal rat, and the cDNAs are converted into single-stranded DNAs using helper phage, followed by subtraction (Proc. Natl. Acad. Sci. USA, 88, 825 (1991)) was used. The subtraction is performed by a method wherein the cDNAs are hybridized to biotinylated mRNAs from the heart of an 8-week-old rat, streptavidin is bound to the hybridized biotinylated mRNA-cDNA complex, and are extracted with phenol.

(1)-1-A. Preparation of cDNA Library From the Heart of a 16-Day-Old Fetal Rat

The guanidine thiocyanate-cesium trifluoroacetate method (Methods in Enzymol., 154, 3 (1987) can be exemplified as a method for preparing total RNA from rat heart.

An mRNA generally contains a poly (A) tail at the 3′ end. Thus, a poly(A)+ RNA can be prepared from total RNA by methods such as the oligo (dT)-immobilized cellulose column method (Molecular Cloning, 2nd Ed.).

Alternatively, mRNA can be also prepared using a kit, such as the Fast Track mRNA Isolation Kit (Invitrogen), the Quick Prep mRNA Purification Kit (Amersham Pharmacia Biotech), or the like.

Methods for constructing a cDNA library from mRNA include those described in Molecular Cloning 2nd Ed.; Current Protocols in Molecular Biology; DNA cloning 1: Core Techniques, A Practical Approach, Second Edition, Oxford University Press (1995); etc. Methods using commercially available kits, such as SuperScript Plasmid System for cDNA Synthesis and Plasmid Cloning (Life Technologies), and ZAP-cDNA Synthesis Kit (Stratagene), are also included.

Cloning vectors include vectors that can replicate in E. coli to a high copy number, which have marker genes for transformation, such as the ampicillin resistance gene and kanamycin resistance gene, as well as a multi-cloning site for cDNA insertion, and which can be converted to a single-stranded DNA by a simple method. Said cloning vectors include phagemid vectors that contain a replication signal IG (intergenic space) derived from an M13 phage, and which are plasmids that can be converted to single-stranded DNA phages by helper phage infection. Specifically, said vectors include pBluescript SK(−) pBluescriptII KS(+), pBS(−), pBC(+) (all of them are from Stratagene) pUC118 (TaKaRa Shuzo), etc. The cloning vectors also include λ phage vectors that can be converted to phagemids by in vivo excision using helper phages. Specific examples are: λ ZAPII, ZAP Express (both are from Stratagene), etc. The above-mentioned in vivo excision, method for converting to a single-stranded DNA phage, and method for purifying single-stranded DNA from phage in the culture supernatant can be performed according to the directions set forth in the manual provided with the respective commercially available vectors.

Any E. coli can be used to introduce a vector containing an insert cDNA, so long as it can express the introduced gene. Specifically, such E. coli includes Escherichia coli XL1-Blue MRF (Stratagene; Strategies, 5, 81 (1992)), Escherichia coli C600 (Genetics, 39, 440 (1954)), Escherichia coli Y1088 (Science, 222, 778 (1983)), Escherichia coli Y1090 (Science, 222, 778 (1983)), Escherichia coli NM522 (J. Mol. Biol., 166, 1 (1983)), Escherichia coli K802 (J. Mol. Biol., 16, 118 (1966)), Escherichia coli JM105 (Gene, 38, 275 (1985)) etc.

In the subtraction, cDNAs are used for the hybridization with mRNAs from the heart of an 8-week-old rat, and the type of phagemid determines which of the two strands of the phagemid is converted to a single-stranded DNA. Thus, to prepare a cDNA library, the procedure of cDNA preparation and the orientation of the insert DNA in the vector have to be designed so that every cDNA clone generates an antisense strand (the strand having the nucleotide sequence complementary to the actual mRNA) as the single-stranded DNA.

For example, as described in the manual of ZAP cDNA synthesis kit from Stratagene, cDNA synthesis with reverse transcriptase is performed using an oligo (dT) primer having an XhoI site at the 5′ end and dNTP containing 5-methyl dCTP, instead of dCTP, as a substrate (which prohibits the synthesized cDNA from internal digestion with XhoI). EcoRI adapters are added to each end of the synthesized cDNA, and then, the resulting DNA is digested with XhoI. The digested cDNA is inserted into the EcoRI/XhoI site of vector XZAPII. According to this method, the EcoRI site of the cDNA always corresponds to the 5′ end and the XhoI site to the 3′ end to place the insert in a fixed orientation within the vector.

The cDNA library obtained by the above described method is converted to phagemid vector pBluescript SK(−) by in vivo excision. Then, single-stranded DNA comprising an antisense strand of the cDNA can be provided by infecting helper phage to the phagemid.

(1)-1-B. Subtraction Using mRNA From the Heart of an 8-Week-Old Rat

Using the cDNA library of the phagemid vector prepared in (1)-1-A, single-stranded DNA phages are released into the culture medium via helper phage infection. The single-stranded cDNAs are recovered and purified from the culture medium. When λ phage vectors are used, the same procedure as described above are used after the conversion of the vectors into phagemids by in vivo excision (Molecular Cloning 2nd Ed.). The purification of single-stranded DNAs can be performed according to the method described in Molecular Cloning 2nd Ed.

Specific procedures, reagent compositions and reaction conditions described in Genes to Cells, 3, 459 (1998) can be used in the subtraction. After the biotinylation of the mRNAs prepared from the heart of an 8-week-old rat by the method described in (1)-1-A with PHOTOPROBE biotin (Vector Laboratories), and such, they are hybridized to the above-mentioned single-stranded cDNAs from the heart of 16-day-old fetal rat. After hybridization, the solution is reacted with streptavidin, which tightly binds to biotin, to increase the hydrophobicity. Then, extraction is carried out by adding phenol thereto. Non-hybridized cDNAs are separated into the aqueous layer, and cDNAs that hybridized to the biotinylated mRNAs are extracted into the phenol layer.

(1)-1-C. Construction of a cDNA Library After Subtraction

The subtracted cDNAs prepared in (1)-1-B are converted to double-stranded DNA using an appropriate primer, which has a nucleotide sequence complementary to the nucleotide sequence of the vector portion, and DNA polymerase, such as BcaBEST (TaKaRa Shuzo) or Klenow fragment, to reconverted the cDNAs to a cDNA library by introducing them to E. coli. A preferred method for introducing DNA into E. coli is electroporation due to its high transformation efficiency.

(1)-2. Differential Hybridization

Complementary DNAs, which correspond to genes whose expression levels are elevated in the heart of a 16-day-old fetal rat, are enriched in the subtracted cDNA library prepared in (1)-1. However, not all of the cDNA clones in the library correspond to genes associated with myocardial cell proliferation. In order to select cDNAs of myocardial cell proliferation-associated genes, the expression levels of mRNAs from the heart are compared between a 16-day-old fetal rat and an 8-week-old rat by Northern hybridization (Molecular Cloning 2nd Ed.) using respective cDNA clones as a probe or by RT (reverse-transcribed)-PCR (PCR Protocols, Academic Press (1990)) using primers based on the nucleotide sequence of the cDNA clones. Then, cDNAs of myocardial cell proliferation-associated genes are selected as cDNAs whose mRNA expression level is higher in either of the two rat hearts. Alternatively, differential hybridization described below enables the inclusive and efficient selection of cDNA clones whose expression levels is higher in either of the two hearts.

First, the subtracted cDNA library provided by the method described in (1)-1 is diluted to a concentration which enables separation of respective colonies. Then, the dilution is plated on an agar medium for cultivation, the colonies are isolated, and each isolated colony is cultured in a liquid culture medium under the same condition cDNA is amplified by PCR using cloning vector-specific oligonucleotide primers and, as a template, cDNA comprised in E. coli of the culture liquid. Then, equal aliquots of the reaction solution are respectively spotted onto two sheets of nylon membrane. Following the denaturation and neutralization of the DNAs on the nylon membranes by the method described in “Molecular Cloning 2nd Ed.”, the DNAs are immobilized on the nylon membranes by ultraviolet-light irradiation. One of the two membranes is hybridized with total mRNA from the heart of a 16-day-old fetal rat as a probe, and the other with total mRNA from the heart from an 8-week-old rat. Hybridization signal intensity of respective clones is compared to select clones whose expression levels vary between the heart of an 8-week-old rat and that of a 16-day-old fetal rat. The colonies corresponding to the selected clones are isolated and separately cultured on a 96-well plate. After the culture media is aliquoted as reaction solution by an automatic micro-aliquoter for 96-well plate, such as Hydra96 (Robbins Scientific), PCR is performed. The procedure enables the easy and rapid preparation of two sheets of identical membranes on which the same amount of DNAs of many clones are blotted. In addition, the spots clearly correspond to the original clones.

As the probe, labeled cDNA prepared from total mRNA by an usual method for labeling DNA probe using reverse transcriptase and random primer can be used. However, as compared to DNA probes, RNA probes hybridize more tightly to DNAs on membranes to give intense signals, and thus are preferable. The labeling of the probe can be done using radioisotopes such as ³²P and ³⁵S or nonradioactive substances such as digoxigenin (DIG) and biotin. In terms of safety, nonradioactive substances are more preferable.

The respective RNA probes derived from the heart of a 16-day-old fetal rat and that of an 8-week-old rat are hybridized to the DNAs on the membranes prepared above, and then probes that hybridized to the DNA of respective colonies are detected. Detection of a hybridized probe is performed by any suitable method adapted to the type of the used label. Highly sensitive and quantitative detection methods include, for example, the use of a radioisotope as a label; autoradiography wherein an X-ray film or imaging plate is directly exposed to the signal; and the use of DIG as a label, wherein alkaline phosphatase-labeled anti-DIG antibody is bound according to the DIG system users' guide (Roche), and then reacted with a substrate that gives alkaline phosphatase-mediated light emission, such as CSPD to expose an X-ray film.

For example, the ratio of mRNA molecules corresponding to a gene whose expression in the heart is higher in either a 16-day-old fetal rat or an 8-week old rat is expected to be higher in either of the probes. Thus, when an equal amount of DNA is blotted on the membranes, more probes bind to a cDNA spot corresponding to the gene. Namely, a cDNA corresponding to a gene whose expression level varies between the heart of a 16-day-old fetal rat and that of-an 8-week-old rat, can be selected by comparing the intensities of hybridization signals of spots on two membranes, that are blotted with the same cDNA clone.

Rat cDNAs obtained as described above include those having the nucleotide sequences of SEQ ID NOs: 1, 3, 5, 7, 9, 11, 13, 15, 17, 19, 21, 23, 25, 27, 29, 32, 33, 35, and 37.

(2) Nucleotide Sequence Analysis of the DNAs:

The nucleotide sequences of cDNAs selected by the above-mentioned method corresponding to genes of which expression levels vary between a 16-day-old fetal rat and an 8-week-old rat, can be determined by commonly used methods for nucleotide sequence analysis, for example, the dideoxy method of Sanger et al. (Proc. Natl. Acad. Sci., USA, 74, 5463 (1977)) or using a DNA sequencer such as 373A DNA sequencer. (Perkin Elmer).

Then, novelty of nucleotide sequence determined by the above-mentioned method can be confirmed by homology search against nucleotide sequence databases, such as GenBank, EMBL, and DDBJ, using a homology search program such as Blast.

(3) Preparation of Full-Length cDNAs:

When a DNA obtained by the above-described method is a partial cDNA, for example, a cDNA fragment further extending to the 5′ direction, as compared with the partial cDNA, can be obtained by 5′-RACE, wherein PCR is carried out using primers based on the nucleotide sequence of the cDNA clone (Proc. Natl. Acad. Sci. USA, 85, 8998 (1988)). Further, a full-length cDNA can be obtained by assembling the cDNA fragment with the original partial cDNA.

Full-length cDNAs of myocardial cell proliferation-associated genes that can be obtained according to the above described method include, for example, DNAs having the nucleotide sequences of SEQ ID NOs: 21, 23, 25, 27, and 30.

Furthermore, once a full-length cDNA nucleotide sequence is revealed as described above, a DNA of interest can be obtained by PCR, using as a template the cDNA or cDNA library synthesized from mRNA and primers prepared based on the nucleotide sequence of the full-length cDNA. Alternatively, a DNA of interest can be prepared by chemical synthesis, using a DNA synthesizer, based on the determined nucleotide sequence of the full-length cDNA. Exemplary DNA synthesizers include model 392 from Perkin Elmer, which utilizes the phosphoramidite method.

(4) Isolation of corresponding genes of human:

Genes corresponding to the rat genes obtained as described above, whose expression levels vary between the hearts of fetal rat and adult rat, are also expected to exist in human. In general, proteins having the same function are highly homologous to each other, even when the proteins originate from different animal species, and further, the nucleotide sequences of genes encoding the proteins tend to exhibit high homology to each other. Thus, human cDNAs of interest may be obtained from a human heart cDNA library by hybridization under slightly stringent conditions using the rat cDNAs as probes. Such slightly stringent conditions can be determined according to the following method.

Although depending on the degree of homology between human cDNA and rat cDNA, slightly stringent conditions can be determined as follows. Human chromosomal DNA is digested with restriction enzymes, and then Southern blotting on the digested DNA is carried out using rat cDNA as a probe under several hybridization conditions with varying degrees of stringency. The most stringent condition of those conditions giving clear hybridization bands is determined as the slightly stringent condition. Specifically, when hybridization buffer without formamide is used, the hybridization is carried out in a hybridization buffer with fixed salt concentration of 1 M changing the hybridization temperature gradually from 68 to 42° C. Membrane wash is carried out using 2×SSC containing 0.5% SDS at the same temperature as used in the hybridization. When a hybridization buffer containing formamide is used, hybridization is carried out at fixed temperature (42° C.) and salt concentration (6×SSC) whereas the formamide concentration is gradually changed within a concentration ranging from 50% to 0%. Membrane wash is carried out using 6×SSC containing 0.5% SDS at 50° C.

Further, nucleotide sequence novelty and homology search is performed by the same method as described in (2) with respect to the nucleotide sequence of rat cDNA obtained in (1) or (3). The search is carried out to select nucleotide sequences of human cDNAs that exhibit 60% homology, preferably 80% or higher homology to the whole protein-coding region of the nucleotide sequence of a rat cDNA. A human cDNA exhibiting high homology is expected to be a cDNA of a human counterpart of a rat gene obtained in (1) or (3). Further, the human cDNA can be amplified by RT-PCR using primers corresponding to the nucleotide sequences of the 5′ end and 3′ end of the human cDNA and, as a template, RNAs extracted from human cells or tissues, preferably heart tissue or cells derived from the heart. In some cases, the nucleotide sequence of a human cDNA found in a database may be only partial sequence or EST, but even in such cases, a full-length human counterpart cDNA of the rat cDNA can be obtained by the same method as described in (3).

Furthermore, the nucleotide sequence of the obtained human cDNA can be analyzed by the same method as described in (2) to determine the amino acid sequence of human protein encoded by the cDNA.

(5) Isolation of Genomic Genes:

A rat or human genomic DNA corresponding to the gene of the present invention can be obtained by plaque hybridization and such, by screening a genomic DNA library prepared using chromosomal DNA isolated from rat or human cells or tissues using the rat or human cDNA obtained in (1) or (4) as a probe, following the method described in Molecular Cloning, 2nd Ed. The exon/intron organization of the genomic gene can be clarified by comparing the nucleotide sequence of genomic DNA to that of the cDNA. Furthermore, the nucleotide sequence of the genomic region responsible for transcriptional regulation, including the promoter and such, of a gene of this invention can be determined using the 5′ end of the cDNA as a probe. Said sequences are useful for analyzing the regulatory mechanism involved in the transcription of the gene of the present invention. Moreover, clones wherein a gene of the present invention on the chromosome has been inactivated or substituted with an arbitrary sequence can be created by a technique of homologous recombination (e.g., Nature, 324, 34-38 (1987); Cell, 51, 503-512 (1987)).

(6) Preparation of Oligonucleotides:

Based on the sequence information of a DNA of the present invention, an oligonucleotide or an antisense oligonucleotide having a partial sequence of the DNA of the present invention can be prepared by usual methods or on DNA synthesizer.

The oligonucleotide or antisense oligonucleotide includes, for example, a sense primer corresponding to the nucleotide sequence of the 5′ end of a nucleotide sequence of an mRNA to be detected, or an antisense primer corresponding to the nucleotide sequence of the 3′ end thereof. However, nucleotides corresponding to uracil of mRNA are thymidine in an oligonucleotide primer. A preferred pair of sense primer and antisense primer include oligonucleotides having 5 to 60 nucleotides whose melting temperatures (Tm) and numbers of nucleotides are not extremely different from each other.

Furthermore, derivatives of the oligonucleotides (hereinafter referred to as “oligonucleotide derivatives”) can be also used as oligonucleotides of the present invention.

The oligonucleotide derivatives include, oligonucleotide derivatives wherein phosphodiester bond is converted to phosphorothioate bond; oligonucleotide derivatives wherein phosphodiester bond is converted to N3′-P5′ phosphoramidate bond; oligonucleotide derivatives wherein ribose-phosphodiester bond is converted to peptide-nucleotide bond; oligonucleotide derivatives wherein uracil is substituted with C-5 propynyluracil; oligonucleotide derivatives wherein uracil is substituted with C-5 thiazole uracil; oligonucleotide derivatives wherein uracil is substituted with C-5 propynylcytosine; oligonucleotide derivatives wherein cytosine is substituted with phenoxazine-modified cytosine; oligonucleotide derivatives wherein ribose is substituted with 2′-O-propylribose; and oligonucleotide derivatives wherein ribose is substituted with 2′-methoxyethoxyribose (Cell Technology, 16, 1463 (1997)).

2. Production of Myocardial Cell Proliferation-Associated Proteins

As required, a DNA fragment with a suitable size containing the coding region for a protein can be prepared based on a full-length cDNA.

A recombinant expression vector to express the protein is constructed by inserting the DNA fragment or the full-length cDNA downstream of the promoter in the expression vector.

The recombinant expression vector is introduced into a host cell that is compatible with the expression vector.

All types of cells can be used as host cells so long as they can express a DNA of interest. Examples include bacterial cells belonging to the genus Escherichia, the genus Serratia, the genus Corynebacterium, the genus Brevibacterium, the genus Pseudomonas, the genus Bacillus, the genus Microbacterium, etc.; yeasts belonging to the genus Kluyveromyces, the genus Saccharomyces, the genus Shizosaccharomyces, the genus Trichosporon, the genus Schwanniomyces, etc.; animal cells; and insect cells.

An expression vector capable of replicating autonomously or being integrated into the chromosome of the host cell and containing a promoter at a suitable position where a DNA of a myocardial cell proliferation-associated gene can be transcribed is used for an expression vector.

When a bacterial cell is used as the host cell as the recombinant expression vectors for a myocardial cell proliferation-associated gene, a recombinant expression vector that is capable of replicating autonomously in the bacterial cell and contain a promoter, a ribosome-binding sequence, a DNA of the myocardial cell proliferation-associated gene, and a transcription terminator sequence is preferably used. The vector may further contain genes that regulate the promoter.

Examples of such expression vectors include pBTrp2, pBTac1, pBTac2 (all the vectors are commercially available from Boehringer-Mannheim); pKK233-2 (Amersham Pharmacia Biotech); pSE280 (Invitrogen); pGEMEX-1 (Promega); pQE-8 (QIAGEN); pKYP10 (Unexamined Published Japanese Patent Application (JP-A) Sho 58-110600); pKYP200 (Agricultural Biological Chemistry, 48, 669 (1984)); pLSA1 (Agric. Biol. Chem., 53, 277 (1989)); pGEL1 (Proc. Natl. Acad. Sci. USA, 82, 4306 (1985)); pBluescript II SK(−) (Stratagene); pGEX (Amersham Pharmacia Biotech); pET-3 (Novagen); pTerm2 (U.S. Pat. No. 4,686,191; U.S. Pat. No. 4,939,094; U.S. Pat. No. 5,160,735); pSupex; pUB110; pTP5; pC194; pEG400 (J. Bacteriol., 172, 2392 (1990)); etc.

It is preferred to use an expression vector wherein the distance of the Shine-Dalgarno sequence that is a ribosome binding sequence, and the initiation codon is appropriately adjusted (e.g., 6 to 18 nucleotides).

Any promoter can be used, so long as it directs the expression in the host cell. Such promoters include, for example, trp promoter (Ptrp), lac promoter (Plac), P_{L} promoter, P_{R} promoter, and T7 promoter derived from E. coli or phage; SPO1 promoter; SPO2 promoter; penP promoter; etc. Artificially designed and modified promoters, such as Ptrp×2 (a promoter wherein two Ptrp promoter units are connected in tandem) tac promoter, letI promoter (Gene, 44, 29 (1986)), lacT7 promoter, etc., can be also used.

The production efficiency of a protein of interest can be improved by replacing the the protein-coding nucleotide sequence of a DNA of myocardial cell proliferation-associated gene of the present invention so with a codon that optimizes expression in particular host cell.

A transcription terminator sequence is not essential for the expression of a DNA of myocardial cell proliferation-associated gene of the present invention; however, it is preferable to arrange a transcription terminator sequence immediately downstream of the structural gene.

Host cells to be used for the present invention include microorganisms belonging to the genus Escherichia, the genus Serratia, the genus Corynebacterium, the genus Brevibacterium, the genus Pseudomonas, the genus Bacillus, the genus Microbacterium, etc.; specifically, for example, Escherichia coli XL1-Blue, Escherichia coli XL2-Blue, Escherichia coli DH1, Escherichia coli MC1000, Escherichia coli KY3276, Escherichia coli W1485, Escherichia coli JM109, Escherichia coli HB101, Escherichia coli No.49, Escherichia coli W3110, Escherichia coli NY49, Bacillus subtilis, Bacillus amyloliquefaciens, Brevibacterium ammoniagenes, Brevibacterium immariophilum ATCC14068, Brevibacterium saccharolyticum ATCC14066, Corynebacterium glutamicum ATCC13032, Corynebacterium glutamicum ATCC14067, Corynebacterium glutamicum ATCC13869, Corynebacterium acetoacidophilum ATCC13870, Microbacterium ammoniaphilum ATCC15354, Pseudomonas sp. D-0110, etc.

Any method for introducing a recombinant expression vector into host cells can be used, so long as it ensures the introduction of a DNA into the above-mentioned host cell. Such methods include, for example, a method utilizing calcium ion (Proc. Natl. Acad. Sci. USA, 69, 2110 (1972)), the protoplast method (JP-A Sho 63-248394), and methods described in the literature (Gene, 17, 107 (1982); Molecular & General Genetics, 168, 111 (1979)).

When a yeast cell is used as the host cell, suitable expression vectors include, for example, YEp13 (ATCC37115), YEp24 (ATCC37051) YCp50 (ATCC37419), pHS19, and pHS15.

Any promoter can be used, so long as it can direct the expression in yeast. Such promoters include, for example, PHO5 promoter, PGK promoter, GAP promoter, ADH promoter, gall promoter, ga110 promoter, heat-shock protein promoter, MFα1 promoter, and CUP1 promoter.

The host cells that can be used in the present invention include Saccharomyces cerevisiae, Schizosaccharomyces pombe, Kluyveromyces lactis, Trichosporon pullulans, Schwanniomyces alluvius, etc.

Any method for introducing a recombinant expression vector into yeast host cells can be used, so long as it ensures the introduction of a DNA into yeast. Such methods include, for example, electroporation (Methods in Enzymol., 194, 182 (1990)), the spheroplast method (Proc. Natl. Acad. Sci. USA, 75, 1929 (1978)), the lithium acetate method (J. Bacteriol., 153, 163 (1983)), and the method described in Proc. Natl. Acad. Sci. USA, 75, 1929 (1978).

When an animal cell is used as the host cell, suitable expression vectors include, for example, pcDNAI (Invitrogen), pcDM8 (Invitrogen), pAGE107 (JP-A Hei 3-22979; Cytotechnology, 3, 133 (1990)), pAS3-3 (JP-A Hei 2-227075), pCDM8 (Nature, 329, 840 (1987)), pcDNAI/Amp (Invitrogen), pREP4 (Invitrogen), pAGE103 (J. Biochem., 101, 1307 (1987)), pAGE210, etc.

Any promoter can be used, so long as it directs the expression in animal cells. Such promoters include, for example, the promoter of the IE (immediate early) gene of cytomegalovirus (human CMV), SV 40 early promoter, retroviral promoter, metallothionein promoter, heat-shock protein promoter, and SRα promoter. Further, the enhancer of the IE gene of human CMV may be used in combination with its promoter.

The host cells to be used in the present invention include Namalwa cell, a human cell line; COS cell, derived from monkey; CHO cell, derived from Chinese hamster; HBT5637 (JP-A Sho 63-299); etc.

Any method for introducing a recombinant expression vector into animal host cells can be used as long as it ensures the introduction of a DNA into animal cells. Such methods include, for example, electroporation (Cytotechnology, 3, 133 (1990)), the calcium phosphate method (JP-A Hei 2-227075), and the lipofection method (Proc. Natl. Acad. Sci. USA, 84, 7413 (1987); Virology, 52, 456 (1973)). Preparation and cultivation of a transformant can be carried out according to the method described in JP-A Hei 2-227075 or JP-A Hei 2-257891.

When an insect cell is used as the host cell, the protein can be expressed, for example, by the method as described in “Baculovirus Expression Vectors, A Laboratory Manual”; “Current Protocols in Molecular Biology, supp. 1-38 (1987-1997)”; Bio/Technology, 6, 47 (1988), or the like.

Specifically, a recombinant gene transfer vector and baculovirus are co-introduced into insect cells to release recombinant viruses into the supernatant of insect cell culture. Then, another batch of insect cells are infected with the recombinant viruses to express the protein.

Suitable gene transfer vectors include, for example, pVL1392, pVL1393, pBlueBacIII (all of these are from Invitrogen), etc.

Baculoviruses that can be used in the present invention include, for example, Autographa californica, a nuclear polyhedrosis virus that is infectious to insects belonging to the family of armyworm.

Insect cell that can be used in the present invention include Sf9 and Sf21 both of which are ovarian cell lines from Spodoptera frugiperda (Baculovirus Expression Vectors, A Laboratory Manual, W. H. Freeman and Company, New York, (1992)); and High5 (Invitrogen) that is an ovarian cell line from Trichoplusia ni; etc.

Methods for co-introducing the above-mentioned recombinant gene transfer vector and baculovirus into insect cells to prepare recombinant viruses include, for example, the calcium phosphate method (JP-A Hei 2-227075) and the lipofection method (Proc. Natl. Acad. Sci. USA, 84, 7413 (1987)).

To express a gene, besides direct expression, a protein can be produced and secreted, or expressed as a fusion protein according to methods described in Molecular Cloning 2nd Ed., etc.

Proteins conjugated with sugars or sugar chains can be obtained by expressing the proteins in yeast, animal cells, or insect cells.

A myocardial cell proliferation-associated protein can be produced by culturing, in a culture medium, a transformant harboring a recombinant DNA that is inserted with a DNA of a myocardial cell proliferation-associated gene; expressing and accumulating the myocardial cell proliferation-associated protein in the culture; and recovering the protein from the culture.

A transformant for the production of a myocardial cell proliferation-associated protein of the present invention can be cultured in a culture medium according to usual methods for culturing host cells (i.e., transformants).

When the transformant of the present invention is a prokaryotic host cell, such as E. coli, or a eukaryotic host cell, such as yeast, culture medium used for culturing the transformant may be any natural or synthetic culture medium containing carbon sources, nitrogen sources, inorganic substances, and others that are assimilated by the host cell (i.e., transformant) and which ensure efficient culture of the transformant.

Any carbon source that is assimilated by the host cell can be used, including glucose, fructose, sucrose, and molasses containing these sugars; carbohydrate, such as starch and starch hydrolysate; organic acids, such as acetic acid and propionic acid; and alcohols, such as ethanol and propanol.

Any nitrogen source can be used, including ammonia, various ammonium salts of inorganic or organic acids, such as ammonium chloride, ammonium sulfate, ammonium acetate, ammonium phosphate; other nitrogen-containing compounds; peptone; meat extract; yeast extract; corn steep liquor; casein hydrolysate; soybean cake and soybean cake hydrolysate; and various fermenting microbial cells and digests thereof.

Any inorganic substance can be used, including potassium dihydrogenphosphate, dipotassium hydrogenphosphate, magnesium phosphate, magnesium sulfate, sodium chloride, ferrous sulfate, manganese sulfate, copper sulfate, calcium carbonate, etc.

Culturing is carried out under an aerobic condition by shaking culture, stirring culture with deep aeration, or the like. Preferred temperature for the culture ranges from 15 to 40° C. Typical culture period ranges from 16 hours to 7 days. The pH of culture medium is maintained within 3.0 to 9.0. The pH is adjusted by inorganic or organic acid, alkaline solution, urea, calcium carbonate, ammonia, or the like.

If required, an antibiotic, such as ampicillin or tetracycline, may be added to the culture medium during culturing.

To culture a transformant containing an expression vector with an inducible promoter, an inducer may be added to the culture medium if required. For example, to culture a transformant containing an expression vector with a lac promoter, isopropyl-β-D-thiogalactopyranoside (IPTG) or its equivalent may be added to the culture medium. To culture a transformant containing an expression vector with a trp promoter, indole acrylic acid (IAA) or its equivalent may be added to the culture medium.

When an animal cell is used as the host cell to provide a transformant, the culture medium to be used for the transformant includes the commonly used RPMI1640 (The Journal of the American Medical Association, 199, 519 (1967)), Eagle's MEM (Science, 122, 501 (1952)), Dulbecco's modified MEM (Virology, 8, 396 (1959)), 199 culture medium (Proceeding of the Society for the Biological Medicine, 73, 1 (1950)), these culture media supplemented with fetal calf serum, etc.

Culturing is typically carried out at pH 6 to 8, at 30 to 40° C., under an atmosphere of 5% carbon dioxide for 1 to 7 days.

If required, antibiotics, such as kanamycin or penicillin, may be added to the culture medium during culturing.

When an insect cell is used as the host cell to provide a transformant, culture medium to be used for the transformant includes commonly used TNM-FH (Pharmingen); Sf-900 II SFM (Life Technologies) ExCell400 and ExCell405 (both are from JRH Biosciences); Grace's Insect Medium (Grace, T. C. C., Nature, 0.195, 788 (1962)); etc.

The culture is typically carried out at pH 6 to 7, at 25 to 30° C. for 1 to 5 days.

If required, antibiotics, such as gentamycin, may be added to the culture medium during culturing.

The myocardial cell proliferation-associated protein can be isolated and purified from the culture of a transformant according to usual methods for protein isolation and purification.

For example, when the protein is produced in a soluble form in cells, the cells are harvested by centrifugation after culturing; suspended in aqueous buffer; and then, cell-free extract is prepared by disrupting the cells with a sonicator, French press, Manton Gaulin homogenizer, DYNO-MILL, etc. The cell-free extract is centrifuged, and then, a purified preparation of the protein can be obtained from the obtained supernatant by commonly used methods for protein isolation and purification, including techniques such as solvent extraction, salting-out with ammonium sulfate or the like, desalting, precipitation with organic solvents, anion-exchange chromatography using a resin such as diethylaminoethyl (DEAE)-Sepharose and DIAION HPA-75 (Mitsubishi Chemical), cation-exchange chromatography using resin such as S-Sepharose FF (Amersham Pharmacia Biotech), hydrophobic chromatography using resin such as butyl-Sepharose and phenyl-Sepharose, gel filtration with molecule sieve, affinity chromatography, chromatofocusing, electrophoresis such as isoelectric focusing, etc. These techniques can be used either alone or in combination.

When the protein is produced as an insoluble matter in cells, the cells are harvested, crushed, centrifuged, and then, the protein insoluble matter can be recovered as a precipitated fraction.

The recovered protein insoluble matter is solubilized with a protein denaturant.

The resulting solution is diluted or dialyzed to decrease the concentration of the protein denaturant in the solution. Thus, the protein refolds into the normal conformation. Then, a purified preparation of the protein can be obtained by the same protein isolation and purification method described above.

When a protein or glycosylated form thereof is secreted extracellularly, the protein or glycosylated form thereof can be recovered from culture supernatant. Specifically, the supernatant is separated from the culture by techniques, such as centrifugation; and then, a purified preparation of the protein can be obtained from the supernatant by the protein isolation and purification method described above.

Such proteins that can be obtained following the method described above include, for example, proteins having the amino acid sequences of SEQ ID NOs: 22, 24, 26, 28, and 31.

Alternatively, a protein of the present invention can also be produced by chemical synthesis methods, such as the Fmoc method (fluorenylmethyloxycarbonyl method) and tBoc method (t-butyloxycarbonyl method). Further, the protein can be synthesized by peptide synthesizer, for example, those from Advanced ChemTech (USA), Perkin-Elmer, Amersham Pharmacia Biotech, Protein Technology Instrument (USA), Synthecell-Vega (USA), PerSeptive (USA) Shimazu, etc.

3. Preparation of Antibodies Specifically Recognizing Myocardial Cell Proliferation-Associated Proteins:

Together with an appropriate adjuvant (e.g., complete Freund's adjuvant, aluminum hydroxide gel, Bordetella pertussis vaccine, etc.) a purified preparation of a full-length protein of the present invention or a partial fragment thereof, or a synthetic peptide having a partial amino acid sequence of a protein of the present invention is administered as an antigen subcutaneously, intravenously or intraperitoneally to a nonhuman mammal, such as rabbit, goat, rat, mouse and hamster, at a dose of about 50 to 100 μg/animal. When a peptide is used as the antigen, it is preferable to use the peptide that is covalently linked to a carrier protein, such as keyhole limpet haemocyanin or bovine thyroglobulin. The antigen peptide can be synthesized in a peptide synthesizer.

The antigen is given 3 to 10 times in total at 1- or 2-week intervals after the first administration. The blood is collected from the venous plexus of the eyeground 3 to −7 days after each administration to examine whether the serum is reactive to the antigen used for immunization by enzyme immunoassay (Enzyme Immunoassay (ELISA): Igakushoin (1976); Antibodies-A Laboratory Manual, Cold Spring Harbor Laboratory (1988)), or the like.

When a serum from a nonhuman mammal shows a sufficient antibody titer against the antigen used for immunization, the nonhuman mammal can be used as a source of the serum or antibody-producing cells.

Polyclonal antibodies can be purified from the serum.

Monoclonal antibodies can be obtained from a hybridoma prepared by fusing an antibody-producing cells with myeloma cells derived from a nonhuman mammal; culturing the hybridoma or transplanting the hybridoma to an animal to develop an ascite carcinoma; and isolating and purifying the antibody from the culture medium or the ascites.

Antibody-producing cells to be used in the present invention include those derived from spleen, lymph node, and peripheral blood. Antibody-producing cells from spleen are particularly preferable.

Preferred myeloma cells to be used in the present invention include mouse cell lines such as P3-X63Ag8-U1 (P3-U1) (Current Topics in Microbiology and Immunology, 18, 1 (1978)), P3-NS1/1-Ag41(NS-1) (European J. Immunology, 6, 511 (1976)), SP2/O-Agl4(SP-2) (Nature, 276, 269 (1978)), P3-X63-Ag8653(653) (J. Immunology, 123, 1548 (1979)), P3-X63-Ag8 (X63) (Nature, 256, 495 (1975)), all of which are mouse (BALB/c-derived) myeloma cell lines that are resistant to 8-azaguanine.

Hybridoma cells can be prepared by the following method:

Antibody-producing cells and myeloma cells are combined and suspended in HAT culture medium (culture medium containing hypoxanthine, thymidine, and aminopterin). Then, the cells are cultured for 7 to 14 days. After cultivation, an aliquot of the culture supernatant is used for assays such as enzyme immunoassay to select hybridomas that produce antibodies reactive to the antigen but not to proteins without the antigen. Subsequently, hybridomas are cloned by the limiting-dilution method. Finally, hybridoma cells constantly showing high antibody titers by enzyme immunoassay are selected as monoclonal antibody-producing hybridomas.

Methods for isolating and purifying polyclonal antibodies or monoclonal antibodies include: centrifugal separation, ammonium sulfate precipitation, caprilic acid precipitation, and column chromatography using DEAE-Sepharose column, anion-exchange column, protein-A column, protein-G column, gel filtration column, etc. These methods can be used either alone or in combination.

4. Preparation of Recombinant Viral Vectors Producing Myocardial Cell Proliferation-Associated Proteins:

A method for preparing a recombinant viral vector to produce a myocardial cell proliferation-associated protein of the present invention in specific human tissues is described in detail below.

Based on a full-length cDNA corresponding to a myocardial cell proliferation-associated gene, a DNA fragment of a suitable size containing the coding region of the protein is prepared if necessary.

A recombinant viral vector is then constructed by inserting the DNA fragment or the full-length cDNA downstream of a promoter in the viral vector.

When an RNA viral vector is used as the vector, the recombinant virus can be created by preparing an RNA fragment homologous to the full-length cDNA for the cardiac muscle proliferation-associated gene and inserting it downstream of the promoter in the virus vector. The RNA fragment may be selected from double-stranded strand, or alternatively may be either sense strand or antisense strand depending on the type of the viral vector. For example, where a retroviral vector is used, an RNA homologous to the sense strand is selected. When a sense viral vector is used, an RNA homologous to the antisense strand is selected.

The recombinant viral vector is introduced into a packaging cell compatible with the vector.

The packaging cells can be any of cells supplying proteins which are required for virus packaging and which are deficient in the recombinant viral vector wherein at least one of the genes encoding the proteins is deleted. For example, human kidney-derived HEK293 cell, mouse fibroblast cell NIH3T3, or the like may be used. Proteins to be supplied by the packaging cell include: retrovirus-derived gag, pol, and env when using a retroviral vector, murine; gag, pol, env, vpr, vpu, vif, tat, rev, and nef derived from HIV virus when using a lentiviral vector; adenoviral E1A and E1B, in case of an adenoviral vector; Rep (p5,.p19, p40) and Vp (Cap), when using an adeno-associated virus.

Viral vectors that are used in the present invention include those that produce recombinant viruses in the above-mentioned packaging cells and have a promoter at a position suitable for the transcription of a DNA of myocardial cell proliferation-associated gene in the target cells. Plasmid vectors that can be used in the present invention include MFG (Proc. Natl. Acad. Sci. USA, 92, 6733-6737 (1995)), pBabePuro (Nucleic Acids Res., 18, 3587-3596 (1990)), LL-CG, CL-CG, CS-CG, CLG (Journal of Virology, 72, 8150-8157 (1998)), pAdex1 (Nucleic Acids Res., 23, 3816-3821 (1995)), etc. Any promoter can be used so long as it directs the expression in human tissues, including, for example, the promoter of IE (immediate early) gene of CMV (human CMV), SV 40 early promoter, retroviral promoter, metallothionein promoter, heat-shock protein promoter, and SRα promoter. Further, an enhancer of IE gene of human CMV may be used along with the promoter.

Methods for introducing the recombinant viral vector into the packaging cells include, for example, the calcium phosphate method (JP-A Hei 2-227075) and lipofection method (Proc. Natl. Acad. Sci. USA, 84, 7413 (1987)).

5. Detection of mRNA of Myocardial Cell Proliferation-Associated Genes:

A method for detecting mRNA of a myocardial cell proliferation-associated gene using a DNA of the gene of the present invention is described below.

DNAs that can be used in the following method include, for example, DNAs having the nucleotide sequences of SEQ ID NOs: 1, 3, 5, 7, 9, 11, 13, 15, 17, 19, 21, 23, 25, 27, 30, 32, 33, 35, and 37 and DNA fragments thereof.

Methods for detecting changes in the expression level or conformation of mRNA of a myocardial cell proliferation-associated gene include, for example, (1) Northern blotting, (2) in situ hybridization, (3) quantitative PCR, (4) differential hybridization, (5) DNA chip method, and (6) RNase protection assay.

Samples that can be used for the analysis by the above-mentioned method include mRNA or total RNA (hereinafter mRNA and total RNA are collectively referred to as “sample-derived RNA”) obtained from biological samples such as heart tissues, serum, and saliva from heart disease patients or healthy persons; or samples of primary culture cells of cells that are obtained from the biological samples and are cultured in an appropriate culture medium in a test tube. In addition, paraffin or cryostat sections prepared from tissues obtained from the biological samples can also be used.

In Northern blotting, changes in the expression level or conformation of mRNA of a myocardial cell proliferation-associated gene can be detected by isolating sample-derived RNA by gel electrophoresis, transferring the isolated. RNA onto a supporting material such as a nylon membrane, carrying out hybridization using a labeled probe prepared from a DNA of the present invention, washing the nylon membrane and detecting the band specific to the mRNA of the myocardial cell proliferation-associated gene. When hybridization is carried out, incubation should be performed under conditions ensuring the formation of a stable hybrid of the probe and mRNA of a gene associated with myocardial cell proliferation in the sample-derived RNA. Highly stringent conditions are preferable for the steps of hybridization and washing, in order to prevent false-positive reactions. Such conditions can be determined by considering various factors, such as temperature, ionic strength, nucleotide composition, length of probe, and formamide concentration. These factors are described, for example, in Molecular Cloning 2nd Ed.

The labeled probe to be used in Northern blotting can be prepared, for example, by incorporating a radioisotope, biotin, fluorescent group, chemiluminescent group, or the like, into a DNA of the present invention or an oligonucleotide designed based on the sequence of the DNA by a known method (nick-translation, random priming or kinasing). The amount of bound labeled probes reflects the mRNA expression level of a myocardial cell proliferation-associated gene. Thus, the level can be determined by quantifying the amount of bound labeled probes. Furthermore, conformational changes of mRNA of a myocardial cell proliferation-associated gene can be detected by analyzing the binding site of the labeled probe.

In situ hybridization is a method for detecting the mRNA expression level of a myocardial cell proliferation-associated gene, which comprises the steps of hybridization and washing using the above-mentioned labeled probe and paraffin or cryostat sections of tissues obtained from a living body. It is preferred to carry out the steps of hybridization and washing under highly stringent conditions to prevent false-positive reactions during in situ hybridization. The conditions can be determined by considering various factors such as temperature, ionic strength, nucleotide composition, length of probe, and formamide concentration. These factors are described, for example, in Current Protocols in Molecular Biology.

Methods for detecting mRNA of a myocardial cell proliferation-associated gene with, such as quantitative PCR, differential hybridization, and DNA-chip method, may comprise the step of synthesizing cDNA from sample-derived RNA using an oligo dT primer or random primer and reverse transcriptase (hereinafter the cDNA is referred to as “sample-derived cDNA”). When the sample-derived RNA is mRNA, both of the above-mentioned primers are usable, whereas the oligo dT primer is used in case of the sample-derived RNA as total RNA.

In quantitative PCR, DNA fragments derived from mRNA of a myocardial cell proliferation-associated gene are amplified by PCR using a sample-derived cDNA as a template and primers designed based on the nucleotide sequence of a DNA of the present invention. The amount of amplified DNA fragments reflects the mRNA expression level of the myocardial cell proliferation-associated gene. Thus, the mRNA level can be quantified by using a DNA encoding actin, G3PDH (glyceraldehyde 3-phosphate dehydrogenase), or the like as an internal control. Further, conformational changes of mRNA of a myocardial cell proliferation-associated gene can be detected by separating the amplified DNA fragments by gel electrophoresis. According to this detection method, it is preferred to use primers that are suitable for specific and efficient amplification of the target sequence. Such suitable primers can be designed by considering conditions where neither inter- nor intra-primer base pairing is formed, and where the primers specifically bind to the target cDNAs at a certain annealing temperature and dissociate from the target cDNAs by denaturation. The quantification of amplified DNA fragments must be carried out within a logarithmic phase of the amplification during PCR. A phase of PCR can be identified by recovering DNA fragments amplified in each reaction cycle and quantitatively analyzing them by gel electrophoresis.

Differential hybridization (Trends in Genetics, 7, 314-317 (1991)) and the. DNA chip method (Genome Research, 6, 639-645 (1996)) are methods for detecting differences in expression levels of mRNA of a myocardial cell proliferation-associated gene, which comprise the step of hybridization and washing on a filter or a base, such as glass slide or silicon, on which a DNA of the present invention has been immobilized, using a sample-derived cDNA as a probe. According to either method, the differences in expression level of mRNA of the myocardial cell proliferation-associated gene between control and target samples can be accurately detected by immobilizing actin gene, G3PDH gene, or the like, as an internal control on the filter or base. Alternatively, the expression level of mRNA of a myocardial cell proliferation-associated gene can be accurately quantified by synthesizing labeled cDNA probes from RNA either derived from control sample or target sample using different labeled dNTPs, and then hybridizing these probes simultaneously on the filter or base.

RNase protection assay can be carried out by the following procedure. First, a promoter sequence, such as T7 promoter or SP6 promoter, is linked to the 3′ end of a DNA of the present invention. A labeled antisense RNA is synthesized by in vitro transcription system that uses RNA polymerase and labeled rNTP. The labeled antisense RNA is annealed to sample-derived RNA. The resulting RNA-RNA hybrid is digested with RNase, and then the RNA protected fragment is detected as a band after gel electrophoresis. The expression level of mRNA corresponding to a myocardial cell proliferation-associated gene can be quantified by measuring the intensity of the obtained band.

6. Detection of Causative Genes of Heart Diseases:

A method for detecting causative genes of heart diseases caused by myocardial degeneration wherein DNAs of the myocardial cell proliferation-associated genes of the present invention are used is described below.

DNAs to be used in the method include, for example, DNAs having the nucleotide sequences of SEQ ID NOs: 1, 3, 5, 7, 9, 11, 13, 15, 17, 19, 21, 23, 25, 27, 30, 32, 33, 35 and 37, and DNA fragments thereof.

The most apparent test for evaluating the presence or absence of a causative mutation of a heart disease, which is located within the locus of a myocardial cell proliferation-associated gene, is a direct comparison of the gene between a control group and a heart disease patient.

Specifically, human biological samples, such as heart tissue, serum and saliva, are collected from a heart disease patient and a healthy person. Alternatively, samples are prepared from primary culture cells established from the biological samples. DNAs are extracted from the biological samples or primary culture cell-derived samples (hereinafter the DNA is referred to as “sample-derived DNA”) The sample-derived DNA can be used directly as the sample DNA. Alternatively, DNAs corresponding to a myocardial cell proliferation-associated gene amplified from the sample-derived DNA using primers designed based on the nucleotide sequence of a DNA of the present invention can also be used as the sample DNA. Alternatively, DNA fragments, comprising a myocardial cell proliferation-associated gene amplified by PCR using sample-derived cDNA as a template and primers designed based on the nucleotide sequence of a DNA of the present invention, can also be used as the sample DNA.

A hetero-duplex formed by the hybridization of a DNA strand containing the wild-type allele to a DNA strand containing the mutant allele can be detected as a method to determine the presence of causative mutation of a heart disease in a DNA of a myocardial cell proliferation-associated gene.

Methods for detecting a hetero-duplex include: (1) polyacrylamide gel electrophoresis (Trends Genet., 7, 5 (1991); (2) hetero-duplex detection method by single-strand conformation polymorphism analysis (Genomics, 16, 325-332 (1993)); (3) the method of chemical cleavage of mismatches (CCM; Human Genetics (1996), Tom Strachan and Andrew P. Read, BIOS Scientific Publishers Limited); (4) the method of enzymatic cleavage of mismatches (Nature Genetics, 9, 103-104 (1996)); and (5) denaturing gradient gel electrophoresis (Mutat. Res., 288, 103-112 (1993)); etc.

According to the hetero-duplex detection method by polyacrylamide gel electrophoresis, a DNA of a myocardial cell proliferation-associated gene is amplified as a fragment shorter than 200 bp using a sample-derived DNA or sample-derived cDNA as a template, and primers designed based on the nucleotide sequence of SEQ ID NO: 1, 3, 5, 7, 9, 11, 13, 15, 17, 19, 21, 23, 25, 27, 30, 32, 33, 35 or 37; and then the DNA fragment is subjected to polyacrylamide gel electrophoresis. When a hetero-duplex is formed due to a mutation in the DNA of the myocardial cell proliferation-associated gene, the mobility of the duplex in the gel is lower than that of the homo-duplex without mutations. Thus, such a hetero-duplex can be detected as an extra band. A special gel (Hydro-link, MDE, etc.) gives a higher resolution. Insertions, deletions, and most of the single-nucleotide substitutions can be detected according to the method when the DNA fragment is shorter than 200 bp. It is preferred to carry out the hetero-duplex analysis on a single sheet of gel in combination with a single-strand conformation polymorphism analysis as described below.

According to the single-strand conformation polymorphism analysis (SSCP analysis), a DNA of a myocardial cell proliferation-associated gene is amplified as a fragment shorter than 200 bp using a sample-derived DNA or sample-derived cDNA as a template and primers designed based on the nucleotide sequence of the DNA of the present invention; and then the amplified DNA is electrophoresed on a non-denaturing polyacrylamide gel after denaturation. The amplified DNA of the myocardial cell proliferation-associated gene can be detected as a band by labeling the primers with a radioisotope or fluorescent dye before DNA amplification. Alternatively, unlabeled amplified products can be visualized by silver-staining. Fragments having mutated nucleotide sequences can be detected based on the difference in electrophoretic mobility by the co-electrophoresis of a control DNA to discriminate the difference of the electrophoretic pattern of the wild type and that of the mutant.

According to the method of chemical cleavage of mismatches (CCM), a DNA fragment of a myocardial cell proliferation-associated gene is amplified using a sample-derived DNA or sample-derived cDNA as a template, and primers designed based on the nucleotide sequence of SEQ ID NO: 1, 3, 5, 7, 9, 11, 13, 15, 17, 19, 21, 23, 25, 27, 30, 32, 33, 35 or 37. Mutations in the nucleotide sequence can be detected by hybridizing the amplified DNA fragment with a labeled DNA that has been prepared by incorporating a radioisotope or fluorescent dye into a DNA of the present invention, and digesting one of the DNA strands at the mismatched position by osmium-tetroxide treatment. The method of CCM is one of the most sensitive detection methods, and is applicable to sample of kilobase-length.

A mismatch can be digested enzymatically by the combined use of RNaseA and, instead of the use of osmium tetroxide described above, another enzyme such as T4 phage resolvase or endonuclease VII that are associated with the repair of intracellular mismatches.

According to denaturing gradient gel electrophoresis (DGGE), a DNA fragment of a myocardial cell proliferation-associated gene is amplified using a sample-derived DNA or sample-derived cDNA as a template, and primers designed based on the nucleotide sequence of SEQ ID NO: 1, 3, 5, 7, 9, 11, 13, 15, 17, 19, 21, 23, 25, 27, 30, 32, 33, 35 or 37. Then, the amplified DNA fragment is electrophoresed on a gel with a concentration gradient of a chemical denaturant or alternatively a temperature gradient. The amplified DNA fragment migrates to a position in the gel where the DNA is denatured to single-stranded chains, and the DNA no longer migrates after denaturation. Mutations can be detected due to the differences in the mobility of the amplified DNA in the gel, depending on the presence of mutations in the DNA of the myocardial cell proliferation-associated gene. The addition of a poly(G:C) tail to primers improve the detection sensitivity.

An alternative method for detecting causative genes of heart diseases includes the protein truncation test (PTT method; Genomics, 20, 1-4 (1994)). According to the test, a frame-shift mutation, splice-site mutation and nonsense mutation, all of which may result in protein deficiency, can be specifically detected. In the PTT method, a specific primer, wherein a T7 promoter sequence and eukaryotic translation initiation sequence are linked to the 5′ end of a DNA having the nucleotide sequence of SEQ ID NO: 1, 3, 5, 7, 9, 11, 13, 15, 17, 19, 21, 23, 25, 27, 30, 33 or 35, is used; next, cDNA is prepared from a sample-derived RNA by reverse transcription-PCR (RT-PCR) using the primer. Proteins can be produced by in vitro transcription and translation using the cDNA. Then, the protein is electrophoresed on a gel. When the position of the protein after electrophoresis corresponds to that of a full-length protein, no mutation resulting in protein deficiency exist in the gene. On the other hand, when the protein is deleted, such a protein migrates to a position which corresponds to a shorter protein than the full-length protein by electrophoresis. Furthermore, the position of protein migration reflects the degree of deletion.

Primers designed based on the nucleotide sequence of a DNA of the present invention can be used in order to determine the nucleotide sequences of sample-derived DNA and sample-derived cDNA. The presence of causative mutations of a heart disease in the sample-derived DNA or sample-derived cDNA can be assessed based on the determined nucleotide sequence.

Mutations located outside the coding region of a myocardial cell proliferation-associated gene may be detected by analyzing non-coding regions such as regions in the vicinity of the gene, introns thereof, and regulatory sequence thereof. Heart diseases caused by mutations in the non-coding regions can be detected by the same method as described above.

A gene, which has been suggested to have a mutation in the non-coding region by the method as described above, can be cloned using, as a hybridization probe, a DNA having the nucleotide sequence of SEQ ID NO: 1, 3, 5, 7, 9, 11, 13, 15, 17, 19, 21, 23, 25, 27, 30, 32, 33, 35 or 37. The mutation in the non-coding region can be found according to any one of the above-mentioned methods.

An identified mutation can be analyzed according to the statistical method described in Handbook of Human Genetics Linkage (The John Hopkins University Press, Baltimore (1994)) to identify the mutation as SNP (Single nucleotide polymorphism) linked to a heart disease. Furthermore, a causative gene of a heart disease may be identified by preparing DNAs from a family whose members have anamneses associated with the heart disease according to the method described above and detecting mutations therein.

7. Methods for Predicting the Onset and Prognosis of Heart Disease Using DNA of Myocardial Cell Proliferation-Associated Gene:

DNAs used in the method include, for example, DNAs having the nucleotide sequences of SEQ ID NOs: 1, 3, 5, 7, 9, 11, 13, 15, 17, 19, 21, 23, 25, 27, 30, 32, 33, 35 and 37, and DNA fragments thereof.

The cause of a heart disease can be identified by detecting gene mutations in any tissues from a human individual. For example, when a mutation exists in the germ line, an individual who has inherited the mutation may have a tendency to be affected with heart disease. The mutation can be detected by testing DNA from any of tissues of the individual. For example, a heart disease can be diagnosed by extracting DNA from cells of collected human blood and detecting gene mutations using the DNA. Alternatively, prenatal diagnosis for a heart disease can be performed by collecting fetal cells, placental cells or amniotic cells, extracting DNA from the cells and detecting gene mutations using the DNA.

Further, the type of heart disease can be diagnosed by preparing DNA from a living tissue from lesions of a patient who has developed a heart disease and detecting alterations in genes. The diagnosis may be useful for selecting drugs to be administered. The DNA of the living tissue can be prepared by taking a tissue of the lesion isolated from the peripheral normal tissues, treating it with trypsin or the like, culturing the cells obtained in an appropriate culture medium, and extracting chromosomal DNA and mRNA from the cultured cells.

DNA obtained from human sample by any one of the above-mentioned methods for the purpose of diagnosing a disease is hereinafter referred to as “diagnostic sample-derived DNA”. Further, cDNA synthesized from RNA which is obtained from human sample by any one of the above-mentioned methods for the purpose of diagnosing a disease is referred to as “diagnostic sample-derived cDNA”.

Using DNA of a myocardial cell proliferation-associated gene and diagnostic sample-derived DNA or diagnostic sample-derived cDNA, a heart disease can be diagnosed according to the methods as described above for detecting a causative gene of heart diseases.

Further, methods for diagnosing heart diseases using DNA of a myocardial cell proliferation-associated gene and diagnostic sample-derived DNA or diagnostic sample-derived cDNA include: (1) the method comprising the detection of restriction enzyme sites; (2) the method using an allele-specific oligonucleotide probe (ASO: allele specific oligonucleotide hybridization); (3) PCR using allele-specific oligonucleotide (ARMS: amplification refractory mutation system); (4) oligonucleotide ligation assay (OLA); (5) PCR-PHFA method (PCR-preferential homoduplex formation assay); and (6) method using an oligo DNA array (Protein, Nucleic Acid and Enzyme, 0.43, 2004-2011 (1998)).

The detection of restriction enzyme sites can be achieved by the following method. When a restriction enzyme site is lost or newly generated due to a single nucleotide alteration, mutation can be simply detected by a procedure which comprises amplifying the diagnostic sample-derived DNA or the diagnostic sample-derived cDNA by PCR using primers designed based on the nucleotide sequence of a DNA of a myocardial cell proliferation-associated gene of the present invention, digesting with restriction-enzyme and comparing the obtained restriction fragments of the DNA with those of a normal person. However, a single-nucleotide alteration is a rare event. Thus, for diagnostic purposes, oligonucleotide probes are designed based on the sequence information of a DNA of a myocardial cell proliferation-associated gene of the present invention in conjunction with the information of the separately identified mutations. Then the oligonucleotide probes are immobilized on a filter and subjected to hybridization to detect the mutations by reverse-dot blotting.

The method using allele-specific oligonucleotide probes (ASO) is characterized by hybridization of a short synthetic DNA probe to only a full-matched nucleotide sequence. Thus, single-nucleotide mutations can be detected readily by this method. Specifically, the oligonucleotide probe is designed based on the nucleotide sequence of a DNA of the present invention and on identified nucleotide mutations. The oligonucleotide probe is immobilized on a filter. Hybridization is carried out using a probe prepared from diagnostic sample-derived DNA or diagnostic sample-derived cDNA by PCR using labeled dNTP and primers designed based on the sequence of the DNA of the present invention. Such reverse-dot blotting is often used for this method with ASO.

According to the reverse-dot blotting, oligonucleotides, which have been designed based on the nucleotide sequence of a DNA of the present invention and on the information of mutations, are synthesized directly on a base, such as glass slide and silicon, as a DNA chip (i.e., a high-density array). Then, a small amount of diagnostic sample-derived DNA or diagnostic sample-derived cDNA is reacted on the DNA chip. This mutation-detection method more simply detects various mutations and thus is suitable for large scale diagnosis.

Nucleotide mutations can also be detected by the following oligonucleotide ligation assay (OLA). OLA is described below in detail.

Two types of oligonucleotides, each of which consist of about 20 nucleotide residues, are prepared. Based on the nucleotide sequence of a DNA of the present invention, the oligonucleotides are designed so as to hybridize to the 5′-end and 3′-end sides of the mutated site, respectively. A DNA fragment of a myocardial cell proliferation-associated gene is amplified by PCR using diagnostic sample-derived DNA or diagnostic sample-derived cDNA as a template and primers designed based on the nucleotide sequence of the DNA of the myocardial cell proliferation-associated gene of the present invention. The above-mentioned two types of oligonucleotides are hybridized to the amplified DNA fragment. After-hybridization, the two oligonucleotides are ligated to each other with DNA ligase. For example, one of the two oligonucleotides is biotinylated and the other is labeled with a different labeling substance, such as digoxigenin, to rapidly detect the ligation reaction. OLA requires neither electrophoresis nor centrifugation. Thus, OLA is a mutation-detection method suitable for effective diagnosis of many samples in a short term.

Alternatively, the following PCR-PHFA method allows convenient and quantitative detection of a small quantity of DNA derived from a mutant gene.

PCR-PHFA method comprises: gene amplification (PCR), liquid-phase hybridization with a very high specificity, and ED-PCR (enzymatic detection of PCR product) which detects PCR products by the same procedure as in ELISA.

An amplification product labeled at both ends is prepared by PCR using a primer pair, wherein one is labeled with dinitrophenyl (DNP) and the other with biotin-labeled, and a DNA of the present invention as a template. The labeled product is combined with an excess amount of 20 to 100 fold of non-labeled amplification product that is obtained using a pair of non-labeled primers, which nucleotide sequences are the same as the labeled primers, and the diagnostic sample-derived DNA or diagnostic sample-derived cDNA as a template. The mixture is heat-denatured and cooled under a mild temperature gradient of about 1° C./5-10 minutes to preferentially form hybrids consisting of perfectly complementary strands. The reconstituted labeled DNA is trapped and adsorbed on a streptavidin-immobilized well via biotin. An enzyme-conjugated anti-DNP antibody is bound to the DNA via DNP to detected the resultant complex by coloring reaction with the enzyme. When no gene having the same sequence as that of the labeled DNA exists in the sample, double-stranded DNAs are preferentially reconstituted from the original pair of labeled DNAs and as a result the color is developed. On the other hand, when genes having the same nucleotide sequence are present, the amount of reconstituted labeled DNA markedly reduces due to the random replacement of the complementary strands which finally result in a marked decrease of color development. This method enables detection and quantification of known mutations and polymorphic genes.

8. Immunological Method for Detecting or Quantifying Myocardial Cell Proliferation-Associated Protein Using Antibody Specifically Recognizing the Myocardial Cell Proliferation-Associated Protein:

Immunological methods for detecting and quantifying a myocardial cell proliferation-associated protein intracellulary or extracellulary expressed by microorganisms, animals, insects or tissues using an antibody (polyclonal or monoclonal antibody) that specifically recognizes the myocardial cell proliferation-associated protein of the present invention include fluorescent antibody method; enzyme immunoassay (ELISA); radioimmunoassay (RIA); immunohistochemistry (ABC method, CSA method, etc.), such as immunohistological staining and immunocytological staining; Western blotting; dot blotting; immunoprecipitation; sandwich ELISA (Monoclonal Antibody—Experimental Manual, Kodansha Scientific (1987); The second series of lectures on biochemical experiments Vol. 5, Immunobiochemical Experiments, Tokyo Kagaku Dozin (1986)).

The fluorescent antibody method comprises the steps of reacting an antibody of the present invention with a microorganism, animal cell, insect cell or tissue intracellulary or extracellulary expressing a myocardial cell proliferation-associated protein, further reacting thereto an anti-mouse IgG antibody or a fragment thereof labeled with a fluorescent substance such as fluorescein isothiocyanate (FITC) and assaying the fluorescent dye in flow cytometer.

The enzyme immunoassay (ELISA) comprises the steps of reacting an antibody of the present invention with a microorganism, animal cell, insect cell or tissue intracellulary or extracellulary expressing a myocardial cell proliferation-associated protein, further reacting thereto an anti-mouse IgG antibody labeled with an enzyme such as peroxidase and biotin or a labeled fragment thereof and assaying the color development with photospectrometer.

The radioimmunoassay (RIA) comprises the steps of reacting an antibody of the present invention with a microorganism, animal cell, insect cell or tissue intracellulary or extracellulary expressing a myocardial cell proliferation-associated protein, further reacting thereto an anti-mouse IgG antibody or a fragment thereof labeled with a radioisotope and measuring the radioactivity with scintillation counter, etc.

Immunohistochemistry such as immunohistological staining and immunocytological staining comprises the steps of reacting an antibody of the present invention with a microorganism, animal cell, insect cell or tissue intracellulary or extracellulary expressing a myocardial cell proliferation-associated protein, further reacting thereto an anti-mouse IgG antibody or a fragment thereof labeled with a fluorescent material such as FITC or an enzyme such as peroxidase and biotin, and observing the label under a microscope.

Western blotting comprises the steps of fractionating extract from mic roorganism, animal cell, insect cell or tissue intracellulary or extracellulary expressing a myocardial cell proliferation-associated protein by SDS-polyacrylamide gel electrophoresis (Antibodies—A Laboratory Manual, Cold Spring Harbor Laboratory, (1988)), transferring the protein from the gel onto a PVDF membrane or nitrocellulose membrane, reacting an antibody of the present invention with the membrane, further reacting thereto an anti-mouse IgG antibody or a fragment thereof labeled with a fluorescent substance such as FITC or an enzyme such as peroxidase and biotin and verifying the label.

Dot blotting comprises the steps of blotting an extract from microorganism, animal cell, insect cell or tissue intracellulary or extracellulary expressing a myocardial cell proliferation-associated protein onto a nitrocellulose membrane, reacting an antibody of the present invention with the membrane, further reacting thereto an anti-mouse IgG antibody or a fragment thereof labeled with a fluorescent substance such as FITC or an enzyme such as peroxidase and biotin and verifying the label.

Immunoprecipitation comprises the steps of reacting an antibody of the present invention with an extract of microorganism, animal cell, insect cell or tissue intracellulary or extracellulary expressing a myocardial cell proliferation-associated protein, adding thereto a carrier that specifically binds to immunoglobulin, such as protein G-Sepharose and precipitating the resulting antigen-antibody complex.

The sandwich ELISA comprises the steps of previously immobilizing one of two antibodies that specifically recognizes a myocardial cell proliferation-associated protein on a plate, wherein each of the antibodies recognizes a separate epitope of the same antigen, labeling the other antibody with a fluorescent substance such as FITC or an enzyme such as peroxidase and biotin, reacting an extract of microorganisms, animal cells, insect cells or tissues intracellulary or extracellulary expressing the protein of this invention with the antibody immobilized plate, reacting the labeled antibody thereto and detecting the labeled substance bound thereto.

9. Diagnosis for Heart Diseases Using Antibody Specifically Recognizing Myocardial Proliferation-Associated Protein:

The determination of the alterations in the expression level and conformation of a myocardial cell proliferation-associated protein expressed in human biological samples and human primary culture cells is useful in determining the risk of future onset of a heart disease as well as the cause of a heart disease already developed.

Methods for diagnosing the diseases by detecting the expression level and conformational alterations of a myocardial cell proliferation-associated protein include the above-mentioned fluorescent antibody method, enzyme immunoassay (ELISA), radioimmunoassay (RIA), immunohistochemistry such as immunohistological staining and immunocytological staining (ABC method, CSA method, etc.), Western blotting, dot blotting, immunoprecipitation, sandwich ELISA, etc.

The samples to be used in the diagnosis by the above-mentioned methods include biological samples per se, such as heart tissue from patient's lesions, blood, serum, urine, feces, and saliva; as well as cells and cell extracts obtained from the biological samples. In addition to these, paraffin or cryostat sections of tissues obtained from the biological samples may be used.

10. Screening of Therapeutic Agents for Heart Diseases Using Myocardial Cell Proliferation-Associated Protein, DNA Encoding the Protein or Antibody Recognizing the Protein:

DNAs to be used in the screening method include, for example, DNA having the nucleotide sequences of SEQ ID NOs: 1, 3, 5, 7, 9, 11, 13, 15, 17, 19, 21, 23, 25, 27, 30, 32, 33, 35 and 37. Proteins to be used in the screening method include, for example, a protein having the amino acid sequence selected from the group consisting of the amino acid sequences of SEQ ID NOs: 22, 24, 26, 28 and 31; or a protein having an amino acid sequence wherein one or several amino acids are deleted, substituted, or added in the amino acid sequence of SEQ ID NO: 22, 24, 26 or 28, and having an activity associated with the healing of a heart disease caused by myocardial degeneration. Antibodies to be used in the screening method include antibodies that recognize the proteins.

Microorganisms, animal cells, and insect cells, which are transformed by introducing a DNA of a myocardial cell proliferation-associated gene of the present invention and produce the myocardial cell proliferation-associated protein or a partial polypeptide of the protein, as well as purified myocardial cell proliferation-associated proteins and purified polypeptides, are all useful for screening agents specifically acting on the myocardial cell proliferation-associated protein.

Such agents obtained by the screening may be useful for treating heart diseases.

One example of the above-mentioned screening methods comprises the step of selecting a target compound specifically binding to microorganisms, animal cells, and insect cells, which are transformed to produce a myocardial cell proliferation-associated protein of the present invention or a partial polypeptide of the protein (hereinafter the transformant is referred to as “transformant for screening”). The specific target compound can be detected by comparing its binding pattern to a non-transformed microorganism, animal cell, or insect cell used as a control. Alternatively, the target compound can be selected by a competitive screening using, as an index, the inhibition of binding between the “transformant for screening” and a compound or protein that binds specifically to the “transformant for screening.”

The purified myocardial cell proliferation-associated protein of the present invention or the purified partial polypeptide of the protein can be used to select target compounds which specifically bind to the myocardial cell proliferation-associated protein. The target compound can be quantified by the above-mentioned immunological method, using an antibody that specifically recognizes the myocardial cell proliferation-associated protein of the present invention. Alternatively, a target compound can be selected by competitive screening using, as an index, the inhibition of binding between a myocardial cell proliferation-associated protein or a myocardial cell proliferation-associated polypeptide and another target compound that binds to the protein or polypeptide.

Another example of the above-mentioned screening methods comprises the steps of synthesizing many partial peptides of a myocardial cell proliferation-associated protein on plastic pins or a solid-phase support in high density and then efficiently selecting compounds or proteins selectively binding to the peptides (WO 84/03564).

An expression-controlling agent, which enhances or suppresses the expression of mRNA of a myocardial cell proliferation-associated gene or a myocardial cell proliferation-associated protein in cells of a cardiac cell line, is also effective to treat heart diseases.

Substances suppressing or enhancing transcription or translation of a myocardial cell proliferation-associated gene can be screened by adding various test compounds to cells of a cardiac cell line and detecting changes in the mRNA expression level of the myocardial cell proliferation-associated gene using a DNA of the myocardial cell proliferation-associated gene of the present invention. Such changes in the mRNA expression level of a myocardial cell proliferation-associated gene can be detected by the above-mentioned PCR, Northern blotting, or RNase protection assay.

Alternatively, substances suppressing or enhancing transcription or translation of a myocardial cell proliferation-associated gene can be screened by adding various test compounds to cells of a cardiac cell line and detecting changes in the expression level of the myocardial cell proliferation-associated protein using an antibody specifically recognizing the myocardial cell proliferation-associated protein of the present invention. Such changes in the expression level of the myocardial cell proliferation-associated protein can be detected by the above-mentioned fluorescent antibody method, enzyme immunoassay (ELISA), radioimmunoassay (RIA), and immunohistochemical staining such as immunohistological staining and immunocytological staining (ABC method, CSA method, etc.), Western blotting, dot blotting, immunoprecipitation, and sandwich ELISA.

The compounds obtained by the above-mentioned methods can be assessed for their therapeutic effects on heart diseases by administering the compounds as therapeutic agents to a heart disease model animals, such as a model rat for cardiac infarction, and measuring cardiac action potential, cardiac rate, or the like, of the animal.

11. Method for Delivering Drugs to the Heart in a Specific Manner Using Antibody Specifically Recognizing Myocardial Proliferation-Associated Protein (Drug Delivery Method):

Any antibody can be used in the method of drug delivery, so long as it can specifically recognize a myocardial cell proliferation-associated protein of the present invention; humanized antibodies are particularly preferred.

The humanized antibodies include human chimeric antibodies, human CDRs (Complementary Determining Region; hereinafter abbreviated as “CDR”), grafted antibodies, etc.

The term “human chimeric antibody” refers to an antibody consisting of the variable region of an antibody heavy chain (hereinafter, the heavy chain and variable region are referred to as “H chain” and “V region”, respectively; and thus the variable region of a heavy chain is referred to as “HV” or “VH”) and antibody light chain (hereinafter, the light chain is referred to as “L chain”; and thus the variable region of light chain is referred to as “LV” or “VL”), both of which are derived from a nonhuman animal, and the constant region of a human antibody heavy chain (hereinafter, the constant region is referred to as “C region”; and thus the constant region of antibody heavy chain is referred to as “CH”) and human antibody light chain (hereinafter also referred to as “CL”). Suitable nonhuman animals include mouse, rat, hamster, rabbit, and others, so long as they can be utilized to prepare hybridomas producing monoclonal antibodies.

A human chimeric antibody of the present invention can be produced by isolating cDNAs encoding VH and VL from a hybridoma producing a monoclonal antibody, which can bind to a myocardial cell proliferation-associated protein of the present invention and neutralize the activity of the protein; constructing a human chimeric antibody expression vector by inserting the respective cDNAs into a host cell expression vector containing genes encoding human antibody CH and human antibody CL; introducing the constructed expression vector into host cells; and expressing the antibody.

Any CH of human immunoglobulins (hereinafter abbreviated as “hIg”) can be used for a human chimeric antibody of the present invention. However, those belonging to the hIgG class are preferable, and further those of any subclasses of hIgG1, hIgG2, hIgG3, and hIgG4 belonging to the hIgG class may be used. On the other hand, any CL may be used for the human chimeric antibody as long as they belong to the hIg, and those belonging to the κ class and λ class may be used.

The human CDR grafted antibody refers to an antibody constructed by grafting the amino acid sequences of CDRs in VH and VL of an antibody from a nonhuman animal into appropriate positions of the VH and VL of a human antibody.

A human CDR grafted antibody of the present invention can be produced by constructing cDNAs encoding V regions wherein the CDR sequences of VH and VL of an human antibody is substituted with VH and VL CDR sequences from a nonhuman antibody that is reactive to a myocardial cell proliferation-associated protein of the present invention, and which can bind to the myocardial cell proliferation-associated protein to neutralize the activity of the protein; inserting the respective cDNAs into expression vectors containing genes encoding the human antibody CH and CL; introducing the constructed expression vectors of human CDR grafted antibody into host cells; and expressing the antibody.

The CH for a human CDR grafted antibody of the present invention can be derived from any antibody belonging to the hIg. However, those belonging to the hIgG class are preferable, and further those of any subclass of hIgG1, hIgG2, hIgG3, and hIgG4 belonging to the hIgG class may be used. Further, a CL to construct the human CDR grafted antibody can be derived from any antibody belonging to the hIg class, and those belonging to the κ class and λ class may be used.

The human antibody originally referred to a natural antibody existing in human body. However, antibodies obtained from a phage library of human antibodies and from transgenic animals producing human antibodies created according to advanced techniques of genetic engineering, cell engineering and developmental engineering are also encompassed by the term.

An antibody existing in human body can be obtained, for example, by the following method.

Human peripheral blood lymphocytes are isolated and immortalized by infecting EB virus, or the like. The cells are then cloned. The resulting lymphocytes producing an antibody of interest are cultured. The antibody can be obtained from the culture.

A phage library of human antibody is a library wherein antibody genes prepared from human B cells are inserted into the phage genome and antibody fragments such as Fab and single-chain antibody are displayed on the phage particles. Phages expressing antibody fragments with the desired antigen binding activity can be recovered from the library using, as an index, the binding activity of the phage to a base immobilized with the antigen. Further, the antibody fragment can be converted into a complete human antibody by genetic engineering.

The human antibody-producing transgenic animal refers to an animal wherein human antibody genes are integrated in the cells thereof. Specifically, a human antibody-producing transgenic animal can be created by introducing human antibody genes into mouse ES cells, transplanting the ES cells into early embryos from another mouse individual, and developing the embryos. Methods for preparing human antibodies from a human antibody-producing transgenic animal include a method which comprises the step of preparing hybridomas producing a human antibody by conventional hybridoma preparation methods for a nonhuman mammal, culturing the hybridomas to produce and accumulate the human antibody in the culture.

Antibody fragments include Fab, Fab′, F(ab′)₂, single-chain antibody, disulfide-stabilized variable region fragment (dsFv), peptide containing CDR.

Fab is an antibody fragment with a molecular weight of about 50,000 having antigen-binding activity wherein the N-terminal half (nearly half) of the H chain and the entire L chain are bridged by a disulfide bond, which is one of the fragments provided by treating IgG with a proteolytic enzyme, papain (cleavage at amino acid residue 224 of the H chain).

The Fab of the present invention can be obtained by treating an antibody that specifically reacts to a protein of the present invention with the proteolytic enzyme papain. Alternatively, the Fab can be obtained by inserting DNAs encoding Fab of an antibody into an expression vector, introducing the vector into a host cell, and expressing the DNAs.

F (ab′)₂ is an antibody fragment with a molecular weight of about 100,000 and having antigen-binding activity. It is one of the fragments provided by treating IgG with a proteolytic enzyme, pepsin (cleavage at amino acid residue 234 of the H chain) which fragment is slightly larger than two Fabs bridged together by a disulfide bond at the hinge region.

The F (ab′)₂ of the present invention can be obtained by treating an antibody that specifically reacts to a protein of the present invention with the proteolytic enzyme pepsin. Alternatively, the F(ab′)₂ can be obtained by inserting DNAs encoding F(ab′)₂ of an antibody into an expression vector, introducing the vector into the host, and expressing the DNAs.

Fab′ is an antibody fragment with a molecular weight of about 50,000 and having an antigen binding activity. It is provided by cleaving the disulfide bond in the hinge region of the above-mentioned F(ab′)₂.

The Fab′ of the present invention can be obtained by treating an antibody that specifically reacts to a protein of the present invention with a reducing agent dithiothreitol. Alternatively, the Fab′ can be obtained by inserting DNAs encoding Fab′ fragment of an antibody into an expression vector, introducing the vector into the host, and expressing the DNAs.

A single-chain antibody (hereinafter also referred as “scFv”) is a VH-P-VL or VL-P-VH polypeptide provided by linking a single VH and a single VL together via an appropriate peptide linker (hereinafter referred as “P”) The VH and VL of the scFv to be used in the present invention can be derived from an antibody that specifically reacts to a protein of the present invention, for example, a humanized antibody or human antibody.

The single-chain antibody of the present invention can be obtained by the following method.

The single-chain antibody can be obtained by preparing cDNAs encoding VH and VL of an antibody that specifically reacts to a protein of the present invention, constructing DNAs encoding the single-chain antibody, inserting the DNAs into an expression vector, introducing the vector into the host, and expressing the DNAs.

A disulfide-stabilized variable-region fragment (hereinafter also referred as “dsFv”) is an antibody fragment consisting of VH and VL, each of which has a cysteine residue substituted for the original amino acid residue. The two polypeptides are connected together at the cysteines which form a disulfide bond. The amino acid residues to be replaced with cysteine can be selected based on the predicted antibody conformation according to the method of Reiter et al. (Protein Engineering, 7, 697 (1994)).

The VH and VL of dsFv to be used in the present invention can be derived from an antibody that specifically reacts to a protein of the present invention, for example, a humanized antibody or human antibody.

The disulfide-stabilized variable-region fragment (dsFv) of the present invention can be obtained by the following method.

The dsFv can be obtained by preparing cDNAs encoding VH and VL of an antibody that specifically reacts to a protein of the present invention, constructing DNAs encoding the dsFv, inserting the DNAs into an expression vector, introducing the vector into the host, and expressing the DNAs.

A peptide containing CDR can be produced by a method of chemical synthesis such as Fmoc method and tBoc method.

Fusion antibodies described below, which are prepared from an antibody of the present invention, can be used in drug delivery wherein agents or proteins are delivered specifically to the heart lesions.

The fusion antibody is an antibody wherein agents such as radioisotope, protein, low-molecular-weight compound are chemically linked or linked by genetic engineering to an antibody that specifically reacts to a protein of the present invention, for example, a humanized antibody, a human antibody or an antibody fragment thereof.

The fusion antibody of the present invention can be produced by chemically linking or linking by genetic engineering an agent such as radioisotope, protein, low-molecular-weight compound to the N-terminal or C-terminal end of H or L chain of an antibody that specifically reacts to a protein of the present invention or alternatively an antibody fragment thereof, an appropriate substituent, side chain, or sugar chain in the antibody or antibody fragment.

The radioisotopes to be used for the fusion antibody include ¹³¹I and ¹²⁵I. Antibodies and antibody fragments can be labeled with the radioisotopes, for example, by chloramine T method, or the like.

The low-molecular-weight compounds used for the fusion antibody of the present invention include alkylating agents, such as nitrogen mustard and cyclophosphamide; antimetabolites, such as 5-fluorouracil and methotrexate; antibiotics, such as daunomycin, bleomycin, mitomycin C, daunorubicin and doxorubicin; plant alkaloids, such as vincristine, vinblastine and vindesine; anticancer agents, such as tamoxifen; hormones, such as dexamethasone (Clinical Oncology (Ed. Japanese Association of Clinical Oncology (1996) Cancer and Chemotherapy)); steroid drugs, such as hydrocortisone and prednisone; non-steroidal drugs, such as aspirin and indomethacin; immunomodulators, such as aurothiomalate and penicillamine; immunosuppressants, such as cyclophosphamide and azathioprine; and anti-inflammatory drugs, such as chlorpheniramine maleate and antihistamic agents, such as clemastine (Inflammation and anti-inflammatory treatment (1982) Ishiyaku Pub., Inc.).

The low-molecular-weight agents can be linked to the above-mentioned antibodies by usual methods. For example, daunomycin can be linked to an antibody by linking the amino groups of daunomycin to the antibody via glutaraldehyde or, alternatively, by linking the amino group of daunomycin to the carboxyl group of the antibody via water-soluble carbodiimide.

Preferred proteins for the fusion antibody include cytokines which activate immune cells and growth-regulating factors for the vascular endothelium, vascular smooth muscle, or the like. Examples of such proteins include human interleukin 2, human granulocyte-macrophage colony stimulating factor, human macrophage colony stimulating factor, human interleukin 12, fibroblast growth factor-2 (FGF-2) and platelet-derived growth factor (PDGF).

A fusion antibody with the protein can be prepared by the following method.

A DNA encoding the fusion antibody is constructed by ligating cDNAs encoding an antibody or an antibody fragment thereof to a cDNA encoding the protein. The fusion antibody can be obtained by inserting the DNA into a prokaryotic or eukaryotic expression vector, introducing the vector into the host prokaryote or eukaryote, and expressing the DNA.

12. Agents for Gene Therapy Containing DNA of Myocardial Cell Proliferation-Associated Gene:

An agent for gene therapy utilizing a viral vector containing a DNA of a myocardial cell proliferation-associated gene of the present invention can be prepared by mixing a recombinant viral vector prepared in Section 4 with a base used for gene therapy agents (Nature Genet., 8, 42 (1994)).

A base for the present gene therapy agents can be any base that is commonly used for injection, including distilled water; salt solutions, such as solution of sodium chloride and mixed solution comprising an inorganic salt and sodium chloride; solution of a sugar, such as mannitol, lactose, dextran and glucose; solution of an amino acid, such as glycine and arginine; mixed solution of organic acid solution or salt solution and glucose solution. Further, according to conventional methods, the base can be combined with an adjuvant, such as osmoregulator, pH modifier, vegetable oil such as sesame oil and soy bean oil, detergent such as lecithin and non-ionic detergent, to prepare them as a solution, suspension or dispersion for injection. By powdering, freeze-drying, or the like, these injections can be prepared as preparations to be dissolved at the time of use. When the agent for gene therapy of the present invention is a liquid, it can be used directly for the treatment. When it is a solid, it is dissolved, as required, in a sterilized base described above immediately before gene therapy. Methods for administering a gene therapy agent of the present invention include local administration method, wherein the agent is delivered from patient's coronary artery to the heart.

A viral vector can be delivered to the heart lesions via gene transfer by applying the method of liposome delivery, a method of direct in vivo gene transfer.

A viral vector can be prepared by combining an appropriately-sized DNA of a myocardial cell proliferation-associated gene of the present invention with a polylysine-conjugated specific antibody to the adenoviral hexon protein, and binding the resulting complex with the adenoviral vector. The viral vector is thus stabilized and reaches the target cells. The vector is incorporated via an endosome into the cell and disassembled in the cell, which allows efficient expression of the gene.

A DNA of a myocardial cell proliferation-associated gene can be delivered to the lesions by a non-viral method of gene transfer.

Such non-viral methods of gene transfer known to those skilled in the art include the calcium phosphate co-precipitation method (Virology, 52, 456-467 (1973); Science, 209, 1414-1422 (1980)), microinjection (Proc. Natl. Acad. Sci. USA, 77,-5399-5403 (1980); Proc. Natl. Acad. Sci. USA, 77, 7380-7384 (1980); Cell, 27, 223-231 (1981); Nature, 294,92-94 (1981)), membrane fusion-mediated transfer using liposome (Proc. Natl. Acad. Sci. USA, 84, 7413-7417 (1987); Biochemistry, 28, 9508-9514 (1989); J. Biol. Chem., 264, 12126-12129 (1989); Hum. Gene Ther., 3, 267-275, (1992); Science, 249, 1285-1288 (1990); Circulation, 83, 2007-2011 (1992)), direct DNA incorporation and receptor-mediated DNA transfer method (Science, 247, 1465-1468 (1990); J. Biol. Chem., 266, 14338-14342 (1991); Proc. Natl. Acad. Sci. USA, 87, 3655-3659 (1991); J. Biol. Chem., 264, 16985-16987 (1989); BioTechniques, 11, 474-485 (1991); Proc. Natl. Acad. Sci. USA, 87, 3410-3414 (1990); Proc. Natl. Acad. Sci. USA, 88, 4255-4259 (1991); Proc. Natl. Acad. Sci. USA, 87, 4033-4037 (1990); Proc. Natl. Acad. Sci. USA, 88, 8850-8854 (1991); Hum. Gene Ther., 3, 147-154 (1991)).

Local incorporation and expression of a gene by tissues has been reported for a tumor treatment involving direct administration of a liposome preparation into the target tissue according to the membrane fusion-mediated transfer method that utilizes liposome (Hum. Gene Ther. 3, 399-410 (1992)). Thus, a similar effect may be expected for heart lesions. The technique of direct DNA incorporation is preferable to direct delivery of a DNA to the heart lesions. A receptor-mediated DNA transfer can be carried out, for example, with a protein ligand conjugated with the DNA (which is normally present as a covalently-closed supercoiled plasmid) via polylysine. The ligand is selected depending on the corresponding ligand receptor expressed on the surface of a target cell or cells in the tissue. Exemplary combinations of the receptor and ligand include, the combination of endothelin (ET)-1 receptor and ET-1. If desired, such a ligand-DNA conjugate can be injected directly to the blood vessel to reach a target tissue where the complex is bound to the receptor and internalized to the cells. To prevent intracellular DNA degradation, adenoviruses are co-infected with the DNA to disrupt the function of endosomes.

13. Therapeutic Agents for Heart Diseases Containing Myocardial Cell Proliferation-Associated Protein:

A myocardial cell proliferation-associated protein of the present invention can be used for reconstructing the cardiac structure and function in various heart diseases caused by myocardial degeneration.

A therapeutic agent for heart diseases, which contains a myocardial cell proliferation-associated protein of the present invention, may comprises the protein alone as the active ingredient. Typically, it is preferable to provide it as a pharmaceutical composition that is prepared by mixing the protein with one or more pharmaceutically acceptable carriers by appropriate methods, which are well known to one skilled in the art of pharmaceutics.

Preferred routes of administration are those that are most effective for the therapy, and include oral administration and parenteral administration such as intraoral, tracheobronchial, intrarectal, subcutaneous, intramuscular, and intravenous administrations. For a protein preparation, intravenous administration is preferred.

The dosage forms of the present agents include nebula, capsule, tablet, granule, syrup, emulsion, suppository, injection, ointment, tape, etc.

Preparations suitable for oral administration include emulsion, syrup, capsule, tablet, powder, granule, etc. For example, liquid preparations such as emulsion and syrup can be prepared using, as an additive, water; sugars such as sucrose, sorbitol and fructose; glycols such as polyethylene glycol and propylene glycol; oils such as sesame oil, olive oil and soy bean oil; preservative such as p-hydroxybenzoic acid esters; flavors such as strawberry flavor and peppermint. Capsules, tablets, powders and granules can be produced using, as an additive, excipient such as lactose, glucose, sucrose and mannitol; disintegrator such as starch and sodium alginate; lubricant such as magnesium stearate and talc; binder such as polyvinyl alcohol, hydroxypropylcellulose, and gelatin; detergent such as fatty acid ester; and plasticizer such as glycerin.

Preparations suitable for parenteral administration include injection, suppository and nebula. An injection can be prepared using a carrier comprising salt solution, glucose solution, or a mixture thereof. A powder injection can be prepared by freeze-drying a protein of the present invention according to conventional methods and adding sodium chloride thereto. A suppository can be prepared using a carrier such as cacao butter, hydrogenated oil, and carboxylic acid.

Further, a nebula can be prepared from a protein of the present invention with or without a carrier or the like that has no irritating effect on recipient's oral and airway mucous membrane and allows dispersion of the protein of the present invention as a fine particle to enhance the absorption thereof.

Specific examples of such carriers are lactose and glycerin. Preparations such as aerosol and dry powder can be provided depending on the properties of the carriers and the protein of the present invention to be used. Further, the illustrated additives for the oral dosage forms can also be added as additives in these parenteral dosage forms.

While the dosage and administration frequency depend on the type of disease to be treated, method of administration, period of treatment, age, weight, etc., typically it is within the range of 10 μg/kg/day to 8 mg/kg/day for an adult individual.

14. Therapeutic Agents for Heart Diseases Containing Antibody Specifically Recognizing Myocardial Cell Proliferation-Associated Protein:

An antibody that specifically recognizes a myocardial cell proliferation-associated protein of the present invention can be used without any modification for treating heart diseases, etc.

A therapeutic agent containing an antibody that specifically recognizes a myocardial cell proliferation-associated protein of the present invention, may comprises the antibody alone as the active ingredient. Typically, it is preferable to provide it as a pharmaceutical, prepared by mixing the antibody with one or more pharmaceutically acceptable carriers by an appropriate method that is well known to one skilled in the art of pharmaceutics. The preparation and administration of the therapeutic agent can be carried out in the same way as for the therapeutic agent containing a myocardial cell proliferation-associated protein described above in Section 13.

The present invention is illustrated in detail below with reference to Examples.

### BRIEF DESCRIPTION OF THE DRAWINGS

FIG. 1 shows the results of Northern analysis for genes whose expression levels differ between the fetal and adult hearts. Panels 1 to 19 show the changes of the expression levels of RHDH-009, -063, -068, -098, -099, -231, -249, -274, -286, -057, -185, -226, -235, -239, -279-1, -309, -100, -140 and -093 between the fetal and adult hearts, respectively. In each blot, the left lane contained 12 μg total RNA from the heart of a 16-day-old fetal rat, and the right lane 12 μg total RNA from the heart of an 8-week-old rat.

### BEST MODE FOR CARRYING OUT THE INVENTION

### EXAMPLE 1

### Preparation of cDNA Library From the Heart of 16-Day-Old Fetal Rat

Heart was excised from a fetus of Wistar rat on the 16th day of pregnancy (Japan SLC), and total RNA was prepared therefrom by the guanidine thiocyanate-cesium trifluoroacetate method (Methods in Enzymology, 154, 3 (1987)). mRNA was obtained as poly(A)+RNA by passing the total RNA through an oligo(dT) cellulose column (Collaborative Research). From the obtained mRNA, a cDNA library, which contained 1.0×10⁶ independent plaques, was prepared using ZAP-cDNA synthesis kit (ZAP-cDNA Synthesis Kit, Stratagene). The preparation of the cDNA library was conducted following the method described in the manual attached to the kit. In this cDNA library, a cDNA is inserted in λphage vector λZAPII (Stratagene) at the XhoI/EcoRI site so that the 5′ end of the cDNA is ligated to the EcoRI site.

### EXAMPLE 2

### Preparation of Subtracted Library

(1) Preparation of Single-Stranded DNA

Along with helper phage ExAssist (Stratagene), the cDNA library (in the form of λphage) from rat heart on the 16th-day of fetal period prepared in Example 1, was transfected to a host cell, Escerichia coli XL1-Blue MRF′ (Stratagene). The portion of phagemid pBluescript SK(−) containing a cDNA was excised as a single-stranded DNA phage from the vector by in vivo excision. The single-stranded DNA phage was released to the culture supernatant. The in vivo excision was performed according to the manual from Stratagene. 700 μl of the culture supernatant (titer: 1.8×10⁵ cfu/11) was added to 7 ml of 10 mM MgSO₄ solution containing 1.8×10¹⁰ cells of Escherichia coli SORL (Stratagene), an ExAssist-resistant host cell. The mixture was cultured at 37° C. for 15 minutes, and then combined with 200 ml of 2×YT culture medium (1.6% bactotryptone, 1% yeast extract). The mixture was cultured at 37° C. for 45 minutes with shaking and the single-stranded DNA phages containing cDNA were infected to the bacterial cells. To the mixture, ampicillin was added at a final concentration of 50 μg/ml, and then culturing was continued at 37° C. for one hour with shaking to proliferate phage-infected E. coli cells alone. The cell count was measured by absorbance at a wavelength of 600 nm. Since the cell count was 8.0×10¹⁰, helper phage R408 (Stratagene) was added to the culture at a multiplicity of infection (m.o.i.) of 10 (7.7×10¹¹ pfu), and the culture was incubated at 37° C. for 7 hours with shaking. Again, the single-stranded DNAs were released to the supernatant. The culture liquid was transferred into a sterilized tube and centrifuged at 10,000 rpm at 4.° C. for 10 minutes. Only supernatants containing phages were recovered by transferring them into a fresh sterilized tube. After re-centrifugation under the same condition, the supernatant was filtered through a sterilizing filter with a pore size of 0.22 mm (Millipore) to completely remove cells. 20 ml of 10× buffer (100 mM Tris-HCl (pH 7. 5), 100 mM magnesium chloride) and 140 units of deoxyribonuclease I (Nippon Gene) were added to the supernatant. The mixture was incubated at 37° C. for 30 minutes, and then ¼ volume of 20% polyethylene glycol (molecular weight=6000)-2.5 M sodium chloride was added thereto. The resulting mixture was mixed well and allowed to stand still at room temperature for 20 minutes. The mixture was centrifuged at 10,000 rpm at 4° C. for 10 minutes to precipitate the phages. After completely removing the supernatant, the phage precipitated was suspended in 400 μl of TE (10 mM Tris-HCl (-pH 8.0), 1 mM EDTA (pH 8.0)). 4 μl of 10% SDS and 625 μg (25 μl) of proteinase K were added to the suspension. Then, the suspension was incubated at 42° C. for one hour. After phenol extraction, phenol-chloroform extraction, and chloroform extraction, the aqueous layer was subjected to ethanol precipitation, which gave 75.0 μg of the single-stranded DNA (vector pBluescript SK(−)) derived from the cDNA library from the heart of 16-day-old fetal rat. (2) Biotinylation of RNA Poly(A)+RNA was prepared from the heart of an 8-week-old rat by the method described in Example 1. 10 μg of the RNA and distilled water were combined in a test tube. The total volume of the solution was 20 μl. 30 μl of 1 μg/μl PHOTOPROBE biotin (Vector Laboratories) was added to the solution in dark. The test tube was uncapped and placed on ice, and then the RNA was biotinylated by irradiation with mercury-lamp light from a height of about 10 cm for 20 minutes.

50 μl solution of 100 mM Tris-HCl (pH 9.5) and 1 mM EDTA (pH 8.0) was added to the reaction solution. 100 μl of water-saturated butanol was added to the mixed solution and then the mixture was stirred vigorously. After the mixture was centrifuged at 14,000 rpm at 4° C. for 5 minutes, the upper butanol layer was removed. The same treatment was repeated three times in total. 100 μl of chloroform was added to the aqueous layer and the mixture was stirred vigorously. After the mixture was centrifuged at 14,000 rpm at 4° C. for 5 minutes, the aqueous layer was transferred into a fresh test tube. The treatment was repeated again, and then the RNA was ethanol-precipitated. The recovered RNA precipitate was dissolved in 20 μl of distilled water and the biotinylation treatment was repeated. The biotinylated RNA was stored at −80° C. at the ethanol-precipitated state for later hybridization.

(3) Subtraction

12.5 μl of 2× reaction buffer (80% formamide, 100 mM HEPES (pH 7.5), 2 mM EDTA (pH 8.0), 0.2% SDS), 2.5 μl of 2.5 M sodium chloride, and 0.5 μg (1 μl) of poly(A) (Amersham Pharmacia Biotech) were added to 0.5 μg (1 μl) of the single-stranded DNA from the cDNA library from the heart of 16-day-old fetal rat prepared in (1). Further an aliquot (corresponding to 10 μg of RNA) of the biotinylated RNA prepared in (2), dissolved in 5 μl of distilled water, was added thereto. After heating at 65° C. for 10 minutes, the mixture was incubated at 42° C. for 2 nights for hybridization.

After the hybridization reaction, 400 μl of buffer (500 mM sodium chloride, 50 mM HEPES (pH 7.5), 2 mM EDTA (pH 8.0)) was added to the reaction solution and then 10 μg (5 μl) of streptavidin (Life Technologies) was added thereto. The mixture was incubated at room temperature for 5 minutes. The complex of streptavidin and biotinylated RNA-cDNA hybrid was removed from the water layer by phenol-chloroform extraction. Again, 10 μg of streptavidin was added to the aqueous layer and the mixture was incubated at room temperature for 5 minutes. After conducting phenol-chloroform extraction twice, the sample was treated with chloroform extraction. The aqueous layer was recovered, and then filtered through a unit-filter ultra-free C3 plus TK (Millipore) to adsorb the cDNA on the filter. After washing, the cDNA was eluted from the filter with 30 μl of l/10 TE (1 mM Tris-HCl (pH 8.0), 0.1 mM EDTA (pH 8.0)) to concentrate and desalt the cDNA. The treatment with the filter was carried out according to the manual from Millipore.

(4) Synthesis and Introduction into E. coli of Double-Stranded DNA

14 μl of distilled water and 1 μl of primer extension primer (2 μg/μl) having the nucleotide sequence of SEQ ID NO: 42 were added to a 15-μl aliquot from the 30 μl of subtracted single-stranded DNA obtained as described above. The mixture was heated at 65° C. for 10 minutes. After annealing of the primer to the single-stranded DNA by allowing the mixture to stand still at room temperature for 5 minutes, 5 μl of 10× reaction buffer (which was attached to BcaBEST Dideoxy Sequencing Kit; Takara Shuzo), 10 μl of 1 mM dNTP mixture, 0.5 μl of 3 μg/μl single-stranded DNA binding protein (USB), 2 μl of 2 units/μl BcaBEST DNA polymerase (Takara Shuzo), and 2.5 μl of distilled water were added thereto. Double-stranded DNA was synthesized by incubating the mixture at 65° C. for one hour. 60 μl of distilled water was added to the reaction solution and the solution was subjected to phenol-chloroform extraction, followed by chloroform extraction. The solution was concentrated with unit-filter ultra-free C3 plus TK by the same method as in (3), and finally the double-stranded DNA was dissolved in 20 μl of TE. A ⅕ aliquot of the double-stranded DNA was introduce into E. coli XL-1 Blue MRF′ by electroporation to prepare a cDNA library (subtracted cDNA library).

### EXAMPLE 3

### Differential Hybridization

(1) Preparation of Array Filter

Colonies were formed on LB-Ap agar medium using the subtracted cDNA library prepared in (4) of Example 2. 9,600 colonies thereof were inoculated on 103 plates with 96 wells each of which contained 100 μl of LB-Ap culture medium. Each colony was inoculated in a single well of the 96-well plates and cultured at 37° C., and then 75 μl of 50% glycerol was added thereto. The cultures were stored at −80° C. (this culture liquid for storage is called “glycerol stock”).

Using a 96-pin replicator, the bacteria were inoculated again from the glycerol stocks to wells of 96-well plates, which contained 100 μl of LB-Ap culture medium in each well. The bacteria were grown at 37° C. overnight by allowing them to stand still. 20-μl aliquots of following reaction solution were added to 96-well PCR plates using automatic dispenser Hydra96, and then trace amounts of the liquids of overnight culture containing E. coli were added thereto. The PCR solution contained 2 μl of 10× reaction buffer (attached to ExTaq), 2 μl of 2.5 mM dNTP, 1 μl of 10 μM T3 HT primer (SEQ ID NO: 39), 1 μl of 10 μM T7 primer (SEQ ID NO: 40), 13.8 μl of distilled water and 0.2 μl of Taq DNA polymerase ExTaq (Takara Shuzo). PCR was performed in a thermal cycler and the profile of thermal cycling was: preheating at 94° C. for 5 minutes; 30 cycles of denaturation at 94° C. for 1 minute, annealing at 64° C. for 1 minute, and extension at 72° C. for 1 minute. The reaction solutions were stored at 4° C. The T3 HT primer and T7 primer were designed based on the specific vector sequences flanking the cDNA insert in order to amplify the cDNA portion.

A 0.5-μl aliquot of each reaction solution was spotted onto NYLON TRANSFER MEMBRANE Hybond N⁺ (Amersham Pharmacia Biotech). The spots were arranged into a lattice-shaped configuration in the same way as on the 96-well plate (12 spots by 8 spots). 384 colonies, which corresponded to four 96-well plates, were spotted onto one sheet of nylon membrane into a lattice-shaped configuration (24 spots by 16 spots). The PCR solution derived from a single colony was spotted onto two sheets of membrane at corresponding positions to obtain the DNA-spotted membranes in duplicate. The membranes on which the reaction solutions had been spotted were air-dried at room temperature, and then placed on paper filters, which had been soaked in denaturation solution (0.5 M NaOH, 1.5 M sodium chloride). The membranes were allowed to stand still at room temperature for 10 minutes. After the DNA was denatured, the membranes were transferred onto paper filters, which had been soaked in neutralization solution (1.0 M Tris-HCl (pH 7.5), 1.5 M sodium chloride) and allowed to stand still at room temperature for 10 minutes. The membranes (array filter) were washed in a square dish filled with sufficient amount of 2× SSC (0.3 M sodium chloride, 30 mM sodium citrate) containing 0.5% SDS.

(2) Probe Labeling

Using Label IT Digoxin Nucleic Acid Labeling Kit (hereinafter referred to as “Label IT Kit”; Mirus), digoxigenin (DIG)-labeled probes were prepared from poly(A)+RNAs obtained from the heart of a 16-day-old fetal rat and the heart from an 8-week old rat prepared in Examples 1 and 2 (2). The labeling was carried out according to the manual attached to the kit.

(3) Hybridization

The methods of hybridization and detection of the hybridized spots, as well as the reagents used therein, were in accordance with the DIG system users' guide from Roche.

The membranes prepared in (1) were placed in a hybridization bag, and then 20 ml of a hybridization buffer (5× SSC, 0.1% N-lauroylsarcosine, 0.02% SDS, 2% blocking reagent (Roche), 50% formamide), which had been preheated at 50° C., was added thereto. The pre-hybridization was carried out at 50° C. for 4 hours. 1/10 volume of denaturation buffer (attached to the Label IT Kit; Mirus) was added to the probe prepared in (2) and incubated at room temperature for 5 minutes. Then 1/10 volume of neutralization buffer (attached to the Label IT Kit; Mirus) was also added thereto. The denatured probe was combined with the hybridization buffer and the mixture was added to the hybridization bag containing the membranes after the pre-hybridization. The bag was incubated for hybridization at 50° C. overnight with shaking so that the filters move in the bag (about 12 rpm). As noted above, the membranes were prepared in duplicate in (1) and thus comprise the same DNA spots. One of the two was hybridized with the probe from the heart of the 16-day old fetal rat, and the other with the probe from the heart of the 8-week-old rat.

(4) Spot Detection

The membranes were taken from the hybridization bag, and then washed with 2× SSC-0.1% SDS at 68° C. for 10 minutes. The membranes were washed again with fresh washing solution under the same condition. Washing was further repeated twice with 0.1× SSC-0.1% SDS at 68° C. for 15 minutes. Then, the membranes were treated with DIG luminescence detection kit (comprising alkaline phosphatase-conjugated anti-DIG antibody and chemiluminescence substrate CSPD (Roche)). X-ray films Hyper Film ECL (Amersham Pharmacia Biotech) were exposed to the luminescent light on the membranes. The films were then developed. The period of exposure was controlled so to reach a similar background level with the probe from the heart of the 16-day old fetal rat and with that of the 8-week-old rat. Clones which exhibited more intense hybridization signals with the probe from the 8-week-old rat heart than with the probe from the heart of the 16-day-old fetal rat, and clones which exhibited more intense hybridization signals with the probe from the heart of the 16-day-old fetal rat than with the probe from the 8-week-old rat heart, were selected. The number of the selected clones was 316 in total. The clones were assigned based on the addresses on the array. Plasmid DNAs of the respective clones were prepared from the cultures obtained from the glycerol stocks prepared in (1) of Example 3.

### EXAMPLE 4

### Analysis of Each Clone

(1) Determination of Nucleotide Sequences

The nucleotide sequences of cDNAs of the 316 clones selected by differential hybridization in Example 3 were determined with a DNA sequencer. These nucleotide sequences were searched for homology against the nucleotide sequences in databases-GenBank, EMBL and GeneSeq (Derwent) using analysis program BlastN. The analysis result demonstrated that known genes, genes encoding slow-fiber troponin I, non-muscle myosin alkali light chain, vimentin and elongation la, were contained among genes that were expressed at higher levels in the fetal heart than in the adult heart.

Next, the entire nucleotide sequences of cDNAs were also determined for the genes whose expression levels were verified to be higher in the heart of the 16-day-old fetal rat as compared with those in the heart of the 8-week-old rat and the genes whose expression levels were verified to be higher in the heart of the 8-week-old rat as compared with those in the heart of the 16-day-old fetal rat by Northern hybridization described below in (2). The amino acid sequences of the proteins encoded by the cDNAs were deduced from the determined nucleotide sequences. Further, these amino acid sequences were also searched for homology against the amino acid sequences in databases SwissProt, PIR, GenPept, TREMBL and GeneSeq using analytical program Blast.

(2) Analysis of Differences in Expression Levels by Northern Hybridization

Clones that seemed to be interesting were selected from the 316 clones isolated in (1). These clones were selected so as to mainly including those having novel nucleotide sequences. Each of the genes was analyzed by Northern hybridization to compare their expression levels in the heart between the 16-day-old fetal rat and the 8-week-old rat.

(2)-1 RNA Transfer onto Membrane

Distilled water was added to 12 μg of total RNA obtained from the heart of 16-day-old fetal rat or heart from 8-week-old rat by the same method as in Example 1 to a total volume of 3.5 μl. 1.5 μl of 10× MOPS buffer (80 mM sodium acetate, 197 mM MOPS, 10 mM EDTA (pH 8.0)) 2 μl of 35% formaldehyde solution (Nacalai Tesque), and 5 μl of deionized formamide were added to the RNA solution. After heating at 65° C. for 5 minutes, the mixture was cooled rapidly on ice. The whole quantity was used for electrophoresis on a 1% agarose gel containing 1×MOPS and 2% formaldehyde. After the electrophoresis, the RNA on the gel was transferred onto NYLON TRANSFER MEMBRANE Hybond N⁺ (Amersham Pharmacia Biotech) by capillary transfer using 20×SSC (3 M sodium chloride, 0.3 M sodium citrate). After the transfer, the RNA was immobilized on the membrane by ultraviolet irradiation in a Cross-Linker Optimal Link (Funakoshi).

(2)-2 Probe Labeling

The selected clones were double-digestion with ApaI and PstI to cut the insert DNA fragments out. The fragments were purified with QIAEX II Gel Extraction Kit (Qiagen) according to the method described in the manual attached to the kit. The DNA fragments were labeled by DIG-High Prime (Roche) using the purified DNA fragments as templates. The labeled DNAs were used as probes. The labeling was conducted in accordance with the manual attached to the kit.

(2).-3 Hybridization and Autoradiography

Methods of hybridization and for detecting the hybridized spots as well as the reagents used were in accordance with the DIG system users' guide from Roche.

The membrane prepared in (1) was placed in a hybridization bag, and then hybridization buffer containing SDS at a high concentration (5×SSC, 0.1% lauroylsarcosyl, 7% SDS, 50 mM sodium phosphate buffer (pH 7.0), 50% formamide, 2% blocking reagent (Roche)) preheated at 50° C. was added thereto. The bag was incubated at 50° C. for several hours or longer for prehybridization. The probe prepared in (2) was denatured by heating at 95° C. for 5 minutes and then was cooled rapidly. The denatured probe was mixed with the hybridization buffer, and the mixture was added to the hybridization bag containing the membranes on which pre-hybridization had been terminated. The bag was incubated for hybridization at 50° C. overnight. The membrane was taken from the hybridization bag, and then washed with 2×SSC-0.1% SDS at 68° C. for 10 minutes. The membrane was washed again with fresh washing solution under the same condition. Further, washing was repeated twice with 0.1×SSC-0.1% SDS at 68° C. for 15 minutes. Then, the membrane was treated with DIG luminescence detection kit, which contains alkaline phosphatase-conjugated anti-DIG antibody and chemiluminescence substrate CSPD (Roche). Then X-ray film Hyper Film ECL (Amersham Pharmacia Biotech) was exposed to the luminescent light on the membrane for autoradiography.

16 clones whose expression levels were higher in the heart of the 16-day-old fetal rat than in the heart of the 8-week-old rat were obtained: RHDH-009, RHDH-063, RHDH-068, RHDH-098, RHDH-099, RHDH-231, RHDH-249, RHDH-274, RHDH-286, RHDH-057, RHDH-185, RHDH-226, RHDH-235, RHDH-239, RHDH-279, and RHDH-309. Three clones whose expression levels were higher in the heart of the 8-week-old rat than in the heart of the 16-day-old fetal rat were obtained: RHDH-100, RHDH-140 and RHDH-093. These clones are described below.

(3) Known Genes Whose Expression Levels are Higher in the Heart of the 16-Day-Old Fetal Rat Than in the Heart of the 8-Week-Old Rat

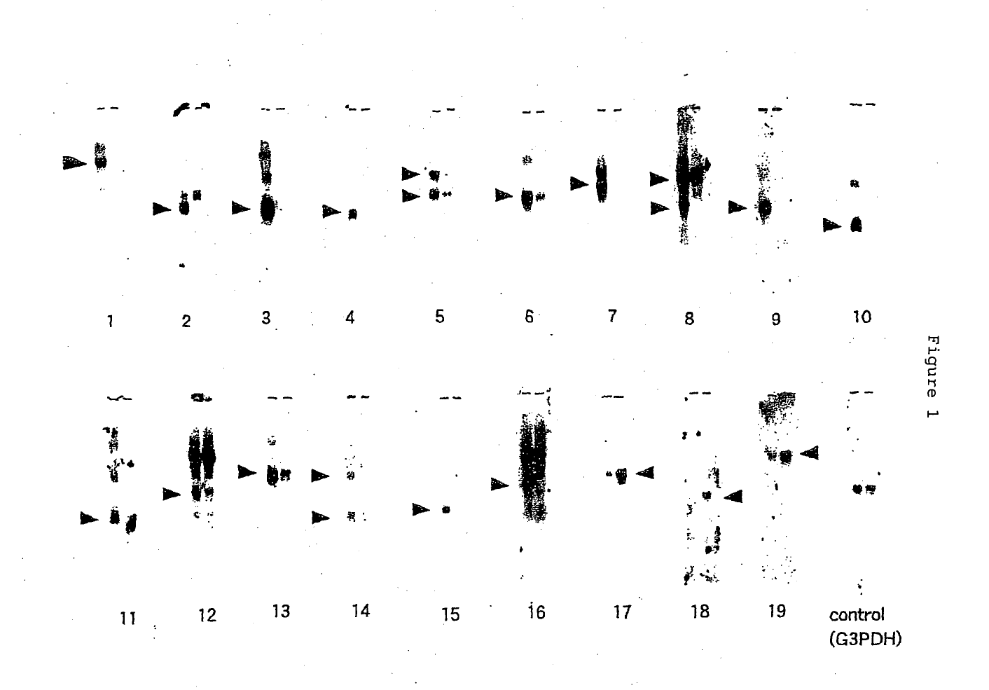

The nucleotide sequence of RHDH-009 was identical with that of rat insulin-like growth factor II (IGF-II) [Accession: X13101] (SEQ ID NO: 1). The amino acid sequence encoded by the gene is shown in SEQ ID NO: 2. IGF-II has been known as a cell-growth factor, but little is known about its physiological function. The result of Northern blotting carried out on the hearts of the 16-day-old fetal rat and the 8-week-old rat is shown in panel 1 of FIG. 1.

The nucleotide sequence of RHDH-063 (SEQ ID NO: 3) exhibited a high (76%) homology to that of the gene encoding a presumptive human secretory protein [Interantional Patent application: WO 99/3126]. Accordingly, RHDH-063 was estimated to be the rat orthologue thereof. The amino acid sequence encoded by the nucleotide sequence of SEQ ID NO: 3 is shown in SEQ ID NO: 4. The protein encoded by the gene exhibited no marked homology to other known proteins, and its function still remains unclear. The result of Northern blotting carried out on the hearts of the 16-day-old fetal rat and the 8-week-old rat is shown in panel 2 of FIG. 1.

The nucleotide sequence of RHDH-068 was identical with that of rat melanocyte-specific expression gene 1 (msgl) [Accession: AF104399] (SEQ ID NO: 5). The amino acid sequence encoded by the gene is shown in SEQ ID NO: 6. The msgl gene has been suggested to participate in cellular pigmentation [Proc. Natl. Acad. Sci. USA, 93, 12298-12303 (1996)]. However, its function in heart remains to be confessed. The result of Northern blotting carried out on the hearts of the 16-day-old fetal rat and the 8-week-old rat is shown in panel 3 of FIG. 1.

The nucleotide sequence of RHDH-098 exhibited high homology to that of mouse c-abl [Accession: L10656] (SEQ ID NO: 7). The amino acid sequence encoded by the gene is shown in SEQ ID NO: 8. The product of c-abl gene has a tyrosine kinase activity. The result of Northern blotting carried out on the hearts of the 16-day-old fetal rat and the 8-week-old rat is shown in panel 4 of FIG. 1.

The nucleotide sequence of RHDH-099 was identical with that of rat non-neuronal enolase [Accession: X02610] (SEQ ID NO: 9). The amino acid sequence encoded by the gene is shown in SEQ ID NO: 10. The non-neuronal enolase gene encodes an enzyme member of the glycolytic system. However, the existence of the difference in the expression level of this gene between the fetal and adult hearts was unknown. The result of Northern blotting carried out on the hearts of the 16-day-old fetal rat and the 8-week-old rat is shown in panel 5 of FIG. 1.

The nucleotide sequence of RHDH-231 was identical with that of rat receptor-linked tyrosine phosphatase (PTP-P1) [Accession: L19180] (SEQ. ID NO: 11). The amino acid sequence encoded by the gene is shown in SEQ ID NO: 12. The product of this gene has a tyrosine phosphatase activity. The result of Northern blotting carried out on the hearts of the 16-day-old fetal rat and the 8-week-old rat is shown in panel 6 of FIG. 1.

The nucleotide sequence of RHDH-249 was identical with that of rat TSC-22 [Accession: L25785] (SEQ ID NO: 13). The amino acid sequence encoded by the gene is shown in SEQ ID NO: 14. It has been reported that the expression of the rat TSC-22 gene is induced by TGF-β [J. Biol. Chem., 267, 10219 (1992)]. However, its function still remains unclear. The result of Northern blotting carried out on the hearts of the 16-day-old fetal rat and the 8-week-old rat is shown in panel 7 of FIG. 1.

The nucleotide sequence of RHDH-274 was identical with that of rat SH3p8 [Accession: AB008161] (SEQ ID NO: 15). The amino acid sequence encoded by the gene is shown in SEQ ID NO: 16. The product of the SH3p8 gene has a Src homology 3 (SH3) domain and an activity to bind to synaptojanin or dynamin I [Proc. Natl. Acad. Sci. USA, 94, 8569-8574 (1997)]. The result of Northern blotting carried out on the hearts of the 16-day-old fetal rat and the 8-week-old rat is shown in panel 8 of FIG. 1.

The nucleotide sequence of RHDH-286 (SEQ ID NO: 17) exhibited high (91%) homology to that of mouse retinoic acid-response protein (MK) [Accession: M35833]. Accordingly, RHDH-286 was estimated to be the rat orthologue of the mouse MK gene. The amino acid sequence encoded by the nucleotide sequence of SEQ ID NO: 17 is shown in SEQ IDNO: 18. It has been known that the expression of the MK gene product is induced at early stages during differentiation of embryonic tumor cells by retinoic acid [Biochem. Biophys. Res. Commun., 151, 1312-1318 (1988)]. The result of Northern blotting carried out on the hearts of the 16-day-old fetal rat and the 8-week-old rat is shown in panel 9 of FIG. 1.

(4) Novel Genes With Higher Expression Level in the Heart of the 16-Day-Old Fetal Rat Than in the Heart of the 8-Week-Old Rat

The nucleotide sequence of RHDH-057 is shown in SEQ ID NO: 19. According to the result of homology analysis, no identical sequence identical to this nucleotide sequence could be found, and thus it was concluded to be a novel sequence. The sequence of RHDH-057 was partially shared by ESTs in UniGene Rn.7790, but no sequence completely covering the nucleotide sequence of RHDH-057 could be found. The amino acid sequence encoded by RHDH-057 is shown in SEQ ID NO: 20. The result of Northern blotting carried out on the hearts of the 16-day-old fetal rat and the 8-week-old rat is shown in panel 10 of FIG. 1.

The nucleotide sequence of RHDH-185 is shown in SEQ ID NO: 21. According to the result of homology analysis, no known genes were found to have a sequence identical to this nucleotide sequence, and thus it was concluded to be a novel sequence. The sequence of RHDH-185 was partially shared by ESTs in UniGene Rn. 12591; however, no sequence was found that completely covered the nucleotide sequence of RHDH-185. The E. coli XL1-Blue MRF′/pRHDH185 strain that contains the plasmid pRHDH-185 containing the cDNA RHDH-185 has been deposited, under the Budapest Treaty, in the following international depositary authority under the accession number FERM BP-7081 on Mar. 10, 2000: International Patent Organism Depositary, National Institute of Advanced Industrial Science and Technology (AIST), Independent Administrative Institution: Chuo 6, 1-1-1 Higashi, Tsukuba, Ibaraki, Japan (Previous Name: The National Institute of Bioscience and Human-Technology, The Agency of Industrial Science and Technology: 1-1-3 Higashi, Tsukuba, Ibaraki, Japan). An ORF of 109 amino acids was found in the nucleotide sequence of RHDH-185. The amino acid sequence is shown in SEQ ID NO: 22. The result of Northern blotting carried out on the hearts of the 16-day-old fetal rat and the 8-week-old rat is shown in panel 11 of FIG. 1.

The nucleotide sequence of RHDH-226 is shown in SEQ ID NO: 23. According to the result of homology analysis, no known genes were found to have a sequence identical to this nucleotide sequence, and thus, it was concluded to be a novel sequence. The sequence of RHDH-226 was partially shared by ESTs in UniGene Rn.7270; however, no sequence was found that completely covered the nucleotide sequence of RHDH-226. The E. coli XL1-Blue MRF′/pRHDH226 strain that contains the plasmid pRHDH-226 containing the cDNA RHDH-226 has been deposited, under the Budapest Treaty, in the following international depositary authority under the accession number FERM BP-7082 on Mar. 10, 2000: International Patent Organism Depositary, National Institute of Advanced Industrial Science and Technology (AIST), Independent Administrative Institution: Chuo 6, 1-1-1 Higashi, Tsukuba, Ibaraki, Japan (Previous Name: The National Institute of Bioscience and Human-Technology, The Agency of Industrial Science and Technology: 1-1-3 Higashi, Tsukuba, Ibaraki, Japan).

An ORF of 376 amino acids was found in the nucleotide sequence of RHDH-226. The amino acid sequence is shown in SEQ ID NO: 24.

Sequences in databases were searched for homology to this amino acid sequence. The result showed that the region of residues 1 to 273 in the amino acid sequence of SEQ ID NO: 24 exhibited high (88%) homology to the amino acid sequence of a putative human secretory protein disclosed in WO 99/06423. However, no amino acid sequence that corresponds to the amino acid sequence of residues 274 to 376 in SEQ ID NO: 24 was found for the protein shown in WO99/06423. Thus, the protein disclosed in WO 99/06423 seems not to be the human orthologue of the protein comprising the amino acid sequence of SEQ ID NO: 24. Furthermore, no functional information on the protein disclosed in WO 99/06423 is available, except that it is a secretory factor. The result of Northern blotting carried out on the hearts of 16-day-old fetal rat and 8-week-old rat is shown in panel 12 of FIG. 1.

The nucleotide sequence of RHDH-235 is shown in SEQ ID NO: 25.

According to the result of homology analysis, this sequence exhibited 65% homology to that of the human metastasis-associated gene 1 (mtal) [Accession: U35113] and 64% homology to that of rat mtal, which is the rat orthologue thereof [Accession: U02522]. An ORF of 513 amino acids is found in the nucleotide sequence of RHDH-235. The amino acid sequence is shown in SEQ ID NO: 26. The amino acid sequence of SEQ ID NO: 26 exhibited 74% homology to the amino acid sequence of the protein encoded by the human mtal gene and 73% homology to that encoded by the rat mtal gene. Since the amino acid sequence of SEQ ID NO: 26 was not identical to neither that of human mtal nor that of rat mtal, RHDH-235 was assumed to be a novel gene. The E. coli XL1-Blue MRF′/pRHDH235 strain that contains the plasmid pRHDH-235 containing the cDNA RHDH-235 has been deposited, under the Budapest Treaty, in the following international depositary authority under the accession number FERM BP-7083 on Mar. 10, 2000: International Patent Organism Depositary, National Institute of Advanced Industrial Science and Technology (AIST), Independent Administrative Institution: Chuo 6, 1-1-1 Higashi, Tsukuba, Ibaraki, Japan (Previous Name: The National Institute of Bioscience and Human-Technology, The Agency of Industrial Science and Technology: 1-1-3 Higashi, Tsukuba, Ibaraki, Japan). The result of Northern blotting carried out on the hearts of the 16-day-old fetal rat and the 8-week-old rat is shown in panel 13 of FIG. 1.

The nucleotide sequence of RHDH-239 is shown in SEQ ID NO: 27. According to the result of homology analysis, no known genes were found to have a sequence identical to this nucleotide sequence, and thus it was concluded to be a novel sequence. The sequence of RHDH-239 was partially shared by ESTs in UniGene Rn.23890, but no sequence was found that completely covered the nucleotide sequence of RHDH-239. The E. coli XL1-BlueMRF′/pRHDH239 that contains the plasmid pRHDH-239 containing the cDNA RHDH-239 has been deposited, under the Budapest Treaty, in the following international depositary authority under the accession number FERM BP-7084 on Mar. 10, 2000: International Patent Organism Depositary, National Institute of Advanced Industrial Science and Technology (AIST), Independent Administrative Institution: Chuo 6, 1-1-1 Higashi, Tsukuba, Ibaraki, Japan (Previous Name: The National Institute of Bioscience and Human-Technology, The Agency of Industrial Science and Technology: 1-1-3 Higashi, Tsukuba, Ibaraki, Japan). An ORF of 158 amino acids was found in the nucleotide sequence of RHDH-239. The amino acid sequence is shown in SEQ ID NO: 28. The result of Northern blotting carried out on the hearts of the 16-day-old fetal rat and the 8-week-old rat is shown in panel 14 of FIG. 1.

The nucleotide sequence of RHDH-279 is shown in SEQ ID NO: 29. According to the result of homology analysis, this sequence exhibited high (92%) homology to that of mouse interferon regulatory factor 3 (mirf3) [Accession: U75840]. A comparison of the nucleotide sequence of SEQ ID NO: 29 and the nucleotide sequence of the mirf3 gene suggested that a part corresponding to the 5′ end of full-length cDNA might be missing in this clone. Thus, a cDNA fragment further extending to the 5′ direction of the cloned cDNA was amplified and isolated by PCR using primers specific to the vector sequence (SEQ ID NO: 39) and specific to RHDH-279 (SEQ ID NO: 41) and the rat cDNA library from the heart of the 16-day-old fetal rat as a template, which was prepared in Example 1 using XZAPII vector.

The RHDH-279-1 clone was obtained by assembling the cDNA fragment and the RHDH-279 cDNA. The nucleotide sequence of RHDH-279-1 is shown in SEQ ID NO: 30. The E. coli XL1-Blue MRF′/PRHDH279-1 that contains the plasmid pRHDH-279-1 containing the cDNA RHDH-279-1 has been deposited, under the Budapest Treaty, in the following international depositary authority under the accession number FERM BP-7085 on Mar. 10, 2000: International Patent Organism Depositary, National Institute of Advanced Industrial Science and Technology (AIST), Independent Administrative Institution: Chuo 6, 1-1-1 Higashi, Tsukuba, Ibaraki, Japan (Previous Name: The National Institute of Bioscience and Human-Technology, The Agency of Industrial Science and Technology: 1-1-3 Higashi-, Tsukuba, Ibaraki, Japan). The sequence segment after the 30th residue in the nucleotide sequence of SEQ ID NO: 30 exhibited high (92%) homology to mirf3. However, the segment of residues 1 to 29 of the nucleotide sequence of SEQ ID NO: 30 was quite different from the sequence of mirf3.

Thus, the nucleotide sequence of SEQ ID NO: 30 is assumed not to be merely the rat orthologue of mifr3. An ORF of 94 amino acids is found in the nucleotide sequence of SEQ ID NO: 30. The amino acid sequence is shown in SEQ ID NO: 31. The result of Northern blotting carried out on the hearts of the 16-day-old fetal rat and the 8-week-old rat is shown in panel 15 of FIG. 1.

The nucleotide sequence of RHDH-309 is shown in SEQ ID NO: 32. According to the result of homology analysis, no sequence of known genes identical to this nucleotide sequence was found, and thus it was concluded to be a novel sequence. The sequence of RHbH-309 was partially shared by ESTs in UniGene Rn.1779, but no sequence completely covering to the nucleotide sequence of RHDH-309 could be found. This sequence had no ORF consisting of 100 amino acids or more, and therefore it was assumed to be a noncoding region. The result of Northern blotting carried out on the hearts of the 16-day-old fetal rat and the 8-week-old rat is shown in panel 16 of FIG. 1.

(5) Known Genes with Higher Expression Level in the Heart of the 8-Week-Old Rat Than in the Heart of the 16-Day-Old Fetal Rat

The nucleotide sequence of RHDH-100 was identical with that of rat protein kinase C receptor [Accession: U03390] (SEQ ID NO: 33).

The amino acid sequence encoded by the gene is shown in SEQ ID NO: 34. The result of Northern blotting carried out on the hearts of the 16-day-old fetal rat and the 8-week-old rat is shown in panel 17 of FIG. 1.

The nucleotide sequence of RHDH-140 (SEQ ID NO: 35) exhibited high (92%) homology to that of mouse pigment epithelium-derived factor (PEDF) [Accession: AF017057]. Thus, RHDH-140 was estimated to be the rat orthologue of the mouse PEDF gene. The amino acid sequence encoded by the nucleotide sequence of SEQ ID NO: 35 is shown in SEQ ID NO: 36. The result of Northern blotting carried out on the hearts of the 16-day-old fetal rat and the 8-week-old rat is shown in panel 18 of FIG. 1.

(6) Novel Genes with Higher Expression Level in the Heart of the 8-Week-Old Rat Than in the Heart of the 16-Day-Old Fetal Rat

The nucleotide sequence of RHDH-093 is shown in SEQ ID NO: 37. According to the result of homology analysis, no known genes were found having a sequence identical to this nucleotide sequence, and thus, it was concluded to be a novel sequence. The sequence of RHDH-093 was partially shared by ESTs in UniGene Rn.16542, but no sequence was found to completely cover the nucleotide sequence of RHDH-093. The amino acid sequence encoded by the nucleotide sequence of RHDH-093 is shown in SEQ ID NO: 38. The result of Northern blotting carried out on the hearts of the 16-day-old fetal rat and the 8-week-old rat is shown in panel 19 of FIG. 1.

### INDUSTRIAL APPLICABILITY

Diagnostic agents and therapeutic agents for various heart diseases caused by myocardial necrosis, for example, hypercardia and cardiac failure, are provided.

“Sequence Listing Free Text”

SEQ ID NO: 39—Description of artificial sequence: synthetic DNA

SEQ ID NO: 40—Description of artificial sequence: synthetic DNA

SEQ ID NO: 41—Description of artificial sequence: synthetic DNA

SEQ ID NO: 42—Description of artificial sequence: synthetic DNA

1. A DNA comprising the nucleotide sequence selected from the group consisting of the nucleotide sequences represented by SEQ ID NOs: 21, 23, 25, 27, and 30.

2. A DNA of a gene that hybridizes, under stringent conditions, to a DNA consisting of the nucleotide sequence represented by SEQ ID NO: 21 or 27, and whose expression level varies between fetal heart and adult heart.

3. A DNA of a gene that hybridizes under stringent conditions to a DNA consisting of the nucleotide sequence represented by SEQ ID NO: 23, 25 or 30, having a 90% or higher homology to the DNA, and whose expression level differs between fetal heart and adult heart.

4. A DNA comprising a sequence that is identical to 5 to 60 consecutive nucleotide residues of the nucleotide sequence selected from the group consisting of the nucleotide sequences represented by SEQ ID NOs: 21, 23, 25, 27 and 30.

5. A DNA comprising a sequence complementary to the DNA according to claim 4.

6. A method for detecting mRNA corresponding to a gene whose expression level varies between fetal heart and adult heart using the DNA according to any one of claims 1 to 5.

7. A diagnostic agent for heart diseases caused by myocardial degeneration, which agent comprises the DNA according to any one of claims 1 to 5.

8. A method for detecting a causative gene of a heart disease caused by myocardial degeneration using the DNA according to any one of claims 1 to 5.

9. A method of screening for a substance suppressing or enhancing transcription or translation of a gene whose expression level varies between fetal heart and adult heart using the DNA according to any one of claims 1 to 5.

10. A method of screening for a therapeutic agent of a heart disease caused by myocardial degeneration using the DNA according to any one of claims 1 to 5.

11. A therapeutic agent for a heart disease caused by myocardial degeneration, wherein the agent comprises the DNA according to any one of claims 1 to 5.

12. A recombinant viral vector comprising the DNA according to any one of claims 1 to 5.

13. A recombinant viral vector comprising an RNA having a sequence homologous to the sense strand of the DNA according to any one of claim 1 to 5.

14. A DNA having the nucleotide sequence selected from the group consisting of the nucleotide sequences represented by SEQ ID NOs: 19, 32, and 37.

15. A DNA of a gene hybridizing under stringent conditions to the DNA according to claim 14, and whose expression level varies between fetal heart and adult heart.

16. A DNA comprising a sequence that is identical to 5 to 60 consecutive nucleotide residues of the nucleotide sequence selected from the group consisting of the nucleotide sequences represented by SEQ ID NOs: 19, 32, and 37.

17. A DNA comprising a sequence complementary to the DNA of claim 16.

18. A diagnostic agent for a heart disease caused by myocardial degeneration, wherein the agent comprises the DNA according to any one of claims 14 to 16.

19. A method for detecting a causative gene of a heart disease caused by myocardial degeneration using the DNA according to any one of claims 14 to 16.

20. A method of screening for a substance suppressing or enhancing transcription or translation of a gene whose expression level varies between fetal heart and adult heart using the DNA according to any one of claims 14 to 16.

21. A method of screening for a therapeutic agent of a heart disease caused by myocardial degeneration using the DNA according to any one of claims 14 to 16.

22. A method for detecting mRNA corresponding to a gene whose expression level varies between fetal heart and adult heart using a DNA having the nucleotide sequence selected from the group consisting of the nucleotide sequences represented by SEQ ID NOs: 1, 3, 5, 7, 9, 11, 13, 15, 17, 33, and 35.

23. A diagnostic agent for a heart disease caused by myocardial degeneration, wherein the agent comprises a DNA comprising the nucleotide sequence selected from the group consisting of the nucleotide sequences represented by SEQ ID NOs: 1, 3, 5, 7, 9, 11, 13, 15, 17, 33, and 35.

24. A method of screening for a substance suppressing or enhancing transcription or translation of a gene whose expression level varies between f etal heart and adult heart using a DNA comprising the nucleotide sequence selected from the group consisting of the nucleotide sequences represented by SEQ ID NOs: 1, 3, 5, 7, 9, 11, 13, 15, 17, 33, and 35.

25. A method of screening for a therapeutic agent of a heart disease caused by myocardial degeneration using a DNA comprising the nucleotide sequence selected from the group consisting of the nucleotide sequences represented by SEQ ID NOs: 1, 3, 5, 7, 9, 11, 13, 15, 17, 33, and 35.

26. A therapeutic agent for a heart disease caused by myocardial degeneration, wherein the agent comprises a DNA comprising the nucleotide sequence selected from the group consisting of the nucleotide sequences represented by SEQ ID NOs: 1, 3, 5, 7, 9, 11, 13, 15, 17, 33, and 35.

27. A recombinant viral vector comprising a DNA having the nucleotide sequence selected from the group consisting of the nucleotide sequences represented by SEQ ID NOs: 1, 3, 5, 7, 9, 11, 13, 15, 17, 33, and 35.

28. A recombinant viral vector comprising an RNA having a sequence homologous to the sense strand of a DNA comprising the nucleotide sequence selected from the group consisting of the nucleotide sequences represented by SEQ ID NOs: 1, 3, 5, 7, 9, 11, 13, 15, 17, 33, and 35.

29. A protein comprising the amino acid sequence selected from the group consisting of the amino acid sequences represented by. SEQ ID NOs: 22, 24, 26, 28, and 31.

30. A protein having an amino acid sequence wherein one or more amino acids are deleted, substituted or added in the amino acid sequence selected from the group consisting of the amino acid sequences represented by SEQ ID NOs: 22, 24, 26, and 28, and which has an activity related to the healing of a heart disease caused by myocardial degeneration.

31. A DNA encoding the protein according to claim 29 or 30.

32. A recombinant DNA that is obtained by inserting the DNA according to any one of claims 1 to 4, and 31 into a vector.

33. A transformant obtained by introducing the recombinant DNA according to claim 32 into a host cell.

34. A method for producing the protein, comprising the steps of culturing the transformant according to claim 33, producing and accumulating the protein according to claim 29 or 30 in the culture, and recovering the protein from the culture.

35. A therapeutic agent for a heart disease caused by myocardial degeneration, which agent comprises the protein according to claim 29 or 30.

36. A method of screening for a therapeutic agent for a heart disease caused by myocardial degeneration comprising the steps of culturing the transformant according to claim 33, and screening the agent using the obtained culture.

37. A method of screening for a therapeutic agent for a heart disease caused by myocardial degeneration using the protein according to claim 29 or 30.

38. A recombinant viral vector associated with the production of the protein according to claim 29 or 30.

39. A therapeutic agent for a heart disease caused by myocardial degeneration, wherein the agent comprises the recombinant viral vector according to claim 38.

40. An antibody recognizing the protein according to claim 29 or 30.

41. An immunological method for detecting the protein of claim 29 or 30 using the antibody according to claim 40.

42. A method of screening for a therapeutic agent for a heart disease caused by myocardial degeneration using the antibody according to claim 40.

43. A method of screening for a substance suppressing or enhancing transcription or translation of a gene whose expression level varies between fetal heart and adult heart using the antibody according to claim 40.

44. A diagnostic agent for a heart disease caused by myocardial degeneration, wherein the agent comprises the antibody according to claim 40.

45. A therapeutic agent for a heart disease caused by myocardial degeneration, wherein the agent comprises the antibody according to claim 40.

46. A drug delivery method for delivering to a cardiac lesion a fusion antibody in which the antibody according to claim 40 is bound to an agent selected from the group consisting of a radioisotope, a protein, and a low-molecular-weight compound.

47. An antibody recognizing a protein comprising the amino acid sequence represented by SEQ ID NO: 20 or 38.

48. A method of screening for a therapeutic agent for a heart disease caused by myocardial degeneration using the antibody according to claim 47.

49. A method of screening for a substance suppressing transcription or translation of a gene whose expression level varies between fetal heart and adult heart using the antibody according to claim 47.

50. A diagnostic agent for a heart disease caused by myocardial degeneration, wherein the agent comprises the antibody according to claim 47.

51. A therapeutic agent for a heart disease caused by myocardial degeneration, wherein the agent comprises the antibody according to claim 47.

52. A drug delivery method for delivering to a cardiac lesion a fusion antibody in which the antibody according to claim 47 is bound to an agent selected from the group consisting of a radioisotope, a protein, and a low-molecular-weight compound.

53. A recombinant viral vector associated with the production of a protein comprising the amino acid sequence selected from the group consisting of the amino acid sequences represented by SEQ ID NOs: 2, 4, 6, 8, 10, 12, 14, 16, 18, 34, and 36.

54. A therapeutic agent for a heart disease caused by myocardial degeneration, which agent comprises the recombinant viral vector according to claim 53.

55. A method of screening for a therapeutic agent for a heart disease caused by myocardial degeneration using an antibody that recognizes a protein comprising the amino acid sequence selected from the group consisting of the amino acid sequences represented by SEQ ID NOs: 2, 4, 6, 8, 10, 12, 14, 16, 18, 34, and 36.

56. A method of screening for a substance suppressing or enhancing transcription or translation of a gene whose expression level varies between fetal heart and adult heart using an antibody that recognizes a protein comprising the amino acid sequence selected from the group consisting of the amino acid sequences represented by SEQ ID NOs: 2, 4, 6, 8, 10, 12, 14, 16, 18, 34, and 36.

57. A diagnostic agent for a heart disease caused by myocardial degeneration, wherein the agent comprises an antibody that recognizes a protein comprising the amino acid sequence selected from the group consisting of the amino acid sequences represented by SEQ ID NOs: 2, 4, 6, 8, 10, 12, 14, 16, 18, 34, and 36.

58. A therapeutic agent for a heart disease caused by myocardial degeneration, wherein the agent comprises an antibody that recognizes a protein comprising the amino acid sequence selected from the group consisting of the amino acid sequences of SEQ ID NOs: 2, 4, 6, 8, 10, 12, 14, 16, 18, 34, and 36.

59. A drug delivery method for delivering to a cardiac lesion a fusion antibody in which an antibody recognizing a protein comprising the amino acid sequence selected from the group consisting of the amino acid sequences represented by SEQ ID NOs: 2, 4, 6, 8, 10, 12, 14, 16, 18, 34, and 36, is bound to an agent selected from the group consisting of a radioisotope, a protein, and a low-molecular-weight compound.
